(19) United States
(12) Patent Application Publication
(10) Pub. No.: US 2013/0195919 A1
(43) Pub. Date: Aug. 1, 2013

(54) INDUCED DENDRITIC CELL COMPOSITIONS AND USES THEREOF

(71) Applicants: Ulrich Von Andrian, Chestnut Hill, MA (US); Roberto Maldonado, Jamaica Plain, MA (US); Fulvia Vascotto, Darmstadt (DE)

(72) Inventors: Ulrich Von Andrian, Chestnut Hill, MA (US); Roberto Maldonado, Jamaica Plain, MA (US); Fulvia Vascotto, Darmstadt (DE)

(73) Assignee: PRESIDENT AND FELLOWS OF HARVARD COLLEGE, Cambridge, MA (US)

(21) Appl. No.: 13/582,897

(22) PCT Filed: Mar. 7, 2011
(86) PCT No.: PCT/US11/27445

§ 371 Date: Nov. 19, 2012

Related U.S. Application Data
(60) Provisional application No. 61/311,023, filed on Mar. 5, 2010.

(51) Int. Cl. C12N 5/0784; G01N 33/50; A61K 35/14

(52) U.S. Cl. CPC C12N 5/0639; A61K 35/15; G01N 33/5079
USPC 424/278.1; 435/325; 435/377; 435/34

(57) ABSTRACT

The invention provides, for example, compositions comprising induced tolerogenic dendritic cells which are capable of suppressing an antigen specific T cell-mediated immune response, and to methods of making and using the same. The invention also provides compositions comprising induced immunogenic dendritic cells and methods of making and using them.

### RELATED APPLICATIONS

This application claims the benefit of priority from U.S. Ser. No. 61/311,023, filed on Mar. 5, 2010, the contents of which are incorporated herein by reference in their entirety.

### GOVERNMENT FUNDING

Work described herein was supported, at least in part, under grants HL056949, AI069259 and A1072252 awarded by the National Institutes of Health. The United States government, therefore, has certain rights in this invention.

### BACKGROUND OF THE INVENTION

An appropriate immune response to an antigen requires a delicate balance between stimulatory and suppressive stimuli. This balance governs responses by various immune cells. Sufficient immune stimulation is necessary in order for a host organism to successfully combat infection by invading pathogens, but an overly abundant immune response can lead to inflammatory damage of host tissue and inappropriate immune responses to host antigens can cause autoimmune disorders.

Since their discovery by Steinman and Cohn in 1973, dendritic cells (DCs) have become increasingly recognized for their crucial role as regulators of innate and adaptive immunity. DCs are exquisitely adept at acquiring, processing, and presenting antigens to T cells. In addition to their role as potent stimulators of adaptive immunity, DCs can prevent, inhibit, or modulate T cell-mediated effector responses through a variety of mechanisms, ranging from the production of pleiotropic anti-inflammatory factors that exert broadly attenuating effects to the induction of antigen-specific T cell responses resulting in anergy, deletion, or induction of regulatory T cells.

Regulatory T cells (Tregs) have pluripotent anti-inflammatory effects on multiple cell types. In particular they control the activation of innate and adaptive immune cells. Tregs acting in an antigen-specific manner reduce effector T cell activation and function, for example, after effector T cells have successfully mounted an attack against an invading pathogen, or to suppress reactivity to self-antigen and thereby prevent autoimmune disease. Two subsets of Tregs are classified according to the location at which they develop in vivo. Naturally occurring Tregs (nTreg) develop in the thymus and suppress self-reactive immune responses in the periphery, whereas adaptive Tregs (aTreg) develop in the periphery from conventional CD4⁺ T cells to ensure tolerance to harmless antigens, including those derived from, for example, food and intestinal flora. Both subsets of Treg cells are characterized by expression of high levels of CD25 and the transcription factor Foxp3. The molecular mechanism by which Tregs exert their suppressive functionality is the subject of intensive research. Currently Tregs are thought to inhibit the antigen-specific expansion and/or activation of self-reactive effector T cells and to secrete suppressive cytokines, including TGFβ or IL-10. The critical role that Tregs play in regulating aberrant immune responses is highlighted by the observation that mutations in either Foxp3 or CD25 lead to multiple lethal autoimmune disorders, including the rapidly fatal autoimmune disorder IPEX (Immune dysregulation, Polyendocrinopathy, Enteropathy X-linked) syndrome. Because of their potential to provide antigen-specific immune regulation without generalized immunosuppression, Tregs have been contemplated for use in cell-based therapy for inflammatory or autoimmune disorders.

An effective means for manipulating dendritic cells to decrease their immunogenicity or increase their tolerogenicity, e.g., their ability to convert naïve T cells to Foxp3⁺ T cells and/or their ability to delete effector T cells has not been identified. The development of such methods for manipulating dendritic cells ex vivo would be of tremendous benefit in promoting or reducing antigen-specific immune responses.

### SUMMARY OF THE INVENTION

The present invention is based, at least in part, on the discovery of induced tolerogenic DCs which possesses at least one of the following characteristics: i) the ability to convert naïve T cells to Foxp3⁺ T regulatory cells ex vivo; ii) the ability to delete effector T cells ex vivo; iii) the ability to retain their tolerogenic phenotype upon stimulation with at least one TLR agonist ex vivo (while, in one embodiment, they increase expression of costimulatory molecules in response to such stimuli); and/or iv) the ability to remain respirostatic upon stimulation with at least one TLR agonist ex vivo. Induced tolerogenic DCs are characterized by antigen specific tolerance induction ex vivo and in vivo and can be generated ex vivo using, e.g., crude, refined, or cell intrinisic antigen sources.

While previous attempts at generating tolerogenic DCs have been made, they have not produced the same type of cells described herein. For example, DCs treated with the mTOR inhibitor Rapamycin alone do not convert naïve CD4⁺CD25⁻ T cells into Treg cells (see, e.g., Turnquist et al. 2007. J. Immunol. 178:7018-7031, first column of page 7027). In marked contrast to these findings, the data presented herein show that induced tolerogenic DCs convert naïve CD4⁺CD25⁻ T cells into Treg cells, even in the absence of cellular proliferation (see, e.g., Example 1). In addition, the authors in Turnquist et al. do not demonstrate actual deletion of effector T cells (e.g., occurring by day 1-2 in culture as demonstrated using induced tolerogenic DCs herein), but rather that DCs treated with rapamycin alone reduce numbers of CD25⁺Fox3⁻ cells as compared to control DC (not treated with rapamycin) in their cultures. In the absence of proof of actual deletion, the effect noted by Turnquist et al. could also result from i) poor capacity of DC treated with rapamycin alone to provide survival signals to T cells and/or ii) robust proliferation induced by control DC (not treated with rapamycin).

In addition to these qualitative differences between induced tolerogenic DCs and DCs treated with rapamycin alone, the protocols described herein for production of induced tolerogenic DCs result in the production of more Treg cells than are obtained using rapamycin alone, thus making production of induced tolerogenic dendritic cells at the scale required for administration to subjects possible.

Others have also attempted to use immature DCs to promote tolerance. However, in contrast to the induced tolerogenic DCs of the instant invention, immature DCs do not maintain their tolerogenic phenotype when exposed to activating stimuli, e.g., TLR agonists. The fact that the cells of the instant invention maintain their tolerogenic phenotype upon such exposure means that crude preparations of antigen, which often contain such stimuli, can be used in the preparation of induced tolerogenic dendritic cells ex vivo.

In addition, protocols for the generation of induced tolerogenic DCs have been developed. In one embodiment, a protocol employs one or more respirostatic agents for treatment of dendritic cells or dendritic cell precursors ex vivo to produce induced tolerogenic DCs capable of antigen specific tolerance induction by i) converting naïve T cells into FoxpP3⁺CD4⁺ regulatory T cells, and/or ii) deleting effector T cells. In another embodiment, a protocol employs at least one agent which tolerogenically locks dendritic cells or dendritic cell precursors ex vivo to produce induced tolerogenic DCs capable of antigen specific tolerance induction by i) converting naïve T cells into FoxpP3⁺CD4⁺ regulatory T cells, and/or ii) deleting effector T cells.

These findings make available, inter alia, methods of making induced tolerogenic dendritic cells ex vivo, compositions of induced tolerogenic dendritic cells, methods of administering these induced tolerogenic dendritic cell compositions, and methods of screening for agents that can be used to make induced tolerogenic dendritic cells.

In addition, the invention pertains to induced immunogenic dendritic cells, which can be used to stimulate effector T cell responses ex vivo or in vivo. Induced immunogenic DCs are more immunogenic than uninduced DCs as can be demonstrated by their ability, e.g., to increase numbers and/or activity of effector T cells ex vivo. In addition, protocols for the generation of induced immunogenic DCs have been developed. In one embodiment, a protocol employs one or more agents for treatment of dendritic cells or dendritic cell precursors ex vivo to produce induced immunogenic DCs capable of promoting antigen specific responses by increasing the activity and/or increasing numbers of effector T cells.

The invention also pertains to methods of making induced immunogenic dendritic cells ex vivo, compositions of induced immunogenic dendritic cells, methods of administering these induced immunogenic dendritic cell compositions, and methods of screening for agents that can be used to make induced immunogenic dendritic cells.

In one aspect, the invention pertains to a composition comprising induced tolerogenic dendritic cells (DCs) which are capable of converting naïve T cells to Foxp3⁺ T regulatory cells ex vivo, wherein the DCs are characterized by antigen specific tolerance induction.

In another aspect, the invention pertains to a composition comprising a population of induced tolerogenic DCs which are capable of deleting effector T cells ex vivo, wherein the DCs are characterized by antigen specific tolerance induction.

In another aspect, the invention pertains to a composition comprising induced tolerogenic DCs which increase expression of costimulatory molecules but retain their tolerogenic phenotype upon stimulation with at least one TLR agonist ex vivo, wherein the DCs are characterized by antigen specific tolerance induction.

In another aspect, the invention pertains to a composition comprising induced tolerogenic DCs which do not transiently increase their oxygen consumption rate upon stimulation with at least one TLR agonist ex vivo, wherein the DCs are characterized by antigen specific tolerance induction.

In one embodiment, the induced tolerogenic DCs are capable of deleting effector T cells ex vivo. wherein the induced tolerogenic DCs are capable of converting naïve T cells to Foxp3 T regulatory cells ex vivo. In one embodiment the induced tolerogenic DCs increase expression of costimulatory molecules but retain their tolerogenic phenotype upon stimulation with at least one TLR agonist ex vivo. In one embodiment the induced tolerogenic DCs do not transiently increase their oxygen consumption rate upon stimulation with at least one TLR agonist ex vivo. In one embodiment the induced tolerogenic DCs are capable of deleting effector T cells ex vivo and increase expression of costimulatory molecules but retain their tolerogenic phenotype upon stimulation with at least one TLR agonist ex vivo.

In one embodiment the induced tolerogenic DCs increase expression of costimulatory molecules but retain their tolerogenic phenotype upon stimulation with at least one TLR agonist ex vivo and do not transiently increase their oxygen consumption rate upon stimulation with at least one TLR agonist ex vivo. In one embodiment the induced tolerogenic DCs increase expression of costimulatory molecules but retain their tolerogenic phenotype upon stimulation with at least one TLR agonist ex vivo and do not transiently increase their oxygen consumption rate upon stimulation with at least one TLR agonist ex vivo. In one embodiment the induced tolerogenic DCs are capable of deleting effector T cells ex vivo and do not transiently increase their oxygen consumption rate upon stimulation with at least one TLR agonist ex vivo. In one embodiment the induced tolerogenic DCs are capable of deleting effector T cells ex vivo and increase expression of costimulatory molecules but retain their tolerogenic phenotype upon stimulation with at least one TLR agonist ex vivo, and do not transiently increase their oxygen consumption rate upon stimulation with at least one TLR agonist ex vivo.

In one embodiment the DCs express class II molecules, and at least a portion of the class II molecules are bound to a plurality of antigenic peptides derived from an antigen to which T cell tolerance is desired.

In another aspect, the invention pertains to a composition comprising induced tolerogenic DCs produced by contacting a starting population of cells comprising dendritic cells or dendritic cell precursors ex vivo with at least one agent that promotes respirostatic tolerance, wherein the DCs are characterized by antigen specific tolerance induction.

In one embodiment the at least one agent is selected from the group consisting of:
i) an mTOR inhibitor and a TGFβ receptor agonist;
ii) a statin;
iii) an mTOR inhibitor and a statin;
iv) an mTOR inhibitor, a TGFβ receptor agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ receptor agonist;
ix) a purinergic receptor antagonist, an mTOR inhibitor, a TGFβ receptor agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

In one aspect, the invention pertains to a composition comprising antigen-specific induced tolerogenic DCs produced by contacting a starting population of cells comprising dendritic cells or dendritic cell precursors ex vivo for less than 10 h with at least one agent selected from the group consisting of: a purinergic receptor antagonist, an mTOR inhibitor, a statin, and an agent which disrupts mitochondrial electron transport in the DCs, wherein the DCs are characterized by antigen specific tolerance induction.

In one embodiment the at least one agent further comprises a TGFβ agonist.

In another aspect, the invention pertains to a composition comprising induced tolerogenic DCs produced by contacting a starting population of cells comprising dendritic cells or dendritic cell precursors ex vivo with at least one agent that causes the DCs to increase expression of costimulatory molecules but retain their tolerogenic phenotype upon stimulation with at least one TLR agonist, wherein the DCs are characterized by antigen specific tolerance induction.

In another aspect, the invention pertains to a composition comprising induced tolerogenic DCs produced by contacting a starting population of cells comprising dendritic cells or dendritic cell precursors ex vivo with at least one agent that causes the DCs to have at least one effect selected from the group consisting of: i) inducing Foxp3 expression in naïve T cells ex vivo, ii) deleting effector T cells or converting FoxP3⁻ effector T cells to FoxP3⁺ effector T cells ex vivo, wherein the DCs are characterized by antigen specific tolerance induction.

In one embodiment, the starting population of cells or the induced tolerogenic DCs are further contacted with an antigen to which T cell tolerance is desired.

In one embodiment, method of administering comprising administering the composition of the invention to a subject.

In one embodiment, the invention pertains method of producing a population of cells comprising induced tolerogenic DCs, the method comprising contacting a starting population of cells comprising dendritic cells or dendritic cell precursors ex vivo with at least one agent that promotes respirostatic tolerance, wherein the DCs are characterized by antigen specific tolerance induction.

In one embodiment, the at least one agent is selected from the group consisting of:
i) an mTOR inhibitor and a TGFβ agonist;
ii) a statin;
iii) an mTOR inhibitor and a statin;
iv) an mTOR inhibitor, a TGFβ agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ agonist;
ix) a purinergic receptor antagonist, an mTOR inhibitor, a TGFβ agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

In one embodiment, the at least one agent is selected from the group consisting of:
i) an mTOR inhibitor and a TGFβ agonist;
ii) a statin;
iii) an mTOR inhibitor, a TGFβ agonist, and a statin;
iv) a purinergic receptor antagonist;
v) an agent which disrupts mitochondrial electron transport in the DCs;

In one embodiment, the at least one agent comprises an mTOR inhibitor and a TGFβ agonist.

In one embodiment, the mTOR inhibitor comprises rapamycin or a derivative or analog thereof.

In one embodiment, the TGFβ agonist is selected from the group consisting of TGFβ1, TGFβ2, TGFβ3, and mixtures thereof.

In one embodiment, the at least one agent comprises a purinergic receptor antagonist.

In one embodiment, the purinergic receptor antagonist binds to a purinergic receptor selected from the group consisting of P1, P2X, P2X7, and P2Y.

In one embodiment, the purinergic receptor antagonist is oxidized ATP.

In one aspect, the invention pertains to a method of producing a population of cells comprising induced tolerogenic DCs the method comprising contacting a starting population of cells comprising dendritic cells or dendritic cell precursors ex vivo for less than 10 h with a composition comprising at least one agent selected from the group consisting of: a purinergic receptor antagonist, an mTOR inhibitor, a TGFβ receptor antagonist, a statin, an agent which disrupts mitochondrial electron transport in the DCs.

In one embodiment, the cells are contacted for 1-3 h. In one embodiment, the cells are contacted for 2 h.

In one embodiment, the DCs are characterized by antigen specific tolerance induction.

In one embodiment, the cells are contacted with at least one agent that promotes respirostatic tolerance.

In one embodiment, the cells are contacted with at least one agent that causes the DCs to increase expression of costimulatory molecules but retain their tolerogenic phenotype upon stimulation with at least one TLR agonist.

In one embodiment, the at least one agent is selected from the group consisting of:
i) an mTOR inhibitor and a TGFβ agonist;
ii) a statin;
iii) an mTOR inhibitor and a statin;
iv) an mTOR inhibitor, a TGFβ agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ agonist;
ix) a purinergic receptor antagonist, an mTOR inhibitor, a TGFβ agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

In one embodiment, the at least one agent is selected from the group consisting of:
i) an mTOR inhibitor and a TGFβ agonist;
ii) a statin;
iii) an mTOR inhibitor, a TGFβ agonist, and a statin;
iv) a purinergic receptor antagonist;
v) an agent which disrupts mitochondrial electron transport in the DCs;

In one embodiment, the at least one agent comprises an mTOR inhibitor and a TGFβ agonist.

In one embodiment, the mTOR inhibitor comprises rapamycin or a derivative or analog thereof.

In one embodiment, the TGFβ agonist is selected from the group consisting of TGFβ1, TGFβ2, TGFβ3, and mixtures thereof.

In one embodiment, the at least one agent comprises a purinergic receptor antagonist.

In one embodiment, the purinergic receptor antagonist binds to a purinergic receptor selected from the group consisting of P1, P2X, P2X7, and P2Y.

In one embodiment, the purinergic receptor antagonist is oxidized ATP.

In one aspect, the invention pertains to a method of producing a population of cells comprising induced tolerogenic DCs, the method comprising contacting a starting population of cells comprising dendritic cells or dendritic cell precursors ex vivo with at least one agent that causes the DCs to increase expression of costimulatory molecules but retain their tolerogenic phenotype upon stimulation with at least one TLR agonist, wherein the DCs are characterized by antigen specific tolerance induction.

In one embodiment, the at least one agent is selected from the group consisting of:
i) an mTOR inhibitor and a TGFβ agonist;
ii) a statin;
iii) an mTOR inhibitor and a statin;
iv) an mTOR inhibitor, a TGFβ agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ agonist;
ix) a purinergic receptor antagonist, an mTOR inhibitor, a TGFβ agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

In one embodiment, the at least one agent is selected from the group consisting of:
i) an mTOR inhibitor and a TGFβ agonist;
ii) a statin;
iii) an mTOR inhibitor, a TGFβ agonist, and a statin;
iv) a purinergic receptor antagonist;
v) an agent which disrupts mitochondrial electron transport in the DCs;

In one embodiment, the at least one agent comprises an mTOR inhibitor and a TGFβ agonist.

In one embodiment, the mTOR inhibitor comprises rapamycin or a derivative or analog thereof.

In one embodiment, the TGFβ agonist is selected from the group consisting of TGFβ1, TGFβ2, TGFβ3, and mixtures thereof.

In one embodiment, the at least one agent comprises a purinergic receptor antagonist.

In one embodiment, the purinergic receptor antagonist binds to a purinergic receptor selected from the group consisting of P1, P2X, P2X7, and P2Y.

In one embodiment, the purinergic receptor antagonist is oxidized ATP.

In one aspect, the invention pertains to a method of producing a population of cells comprising induced tolerogenic DCs, the method comprising contacting a starting population of cells comprising dendritic cells or dendritic cell precursors ex vivo with at least one agent that causes the DCs to have at least one effect selected from the group consisting of: i) inducing Foxp3 expression in naïve T cells ex vivo, ii) deleting effector T cells or converting FoxP3⁻ effector T cells to FoxP3⁺ effector T cells ex vivo, wherein the DCs are characterized by antigen specific tolerance induction.

In one embodiment, the at least one agent is selected from the group consisting of:
i) an mTOR inhibitor and a TGFβ agonist;
ii) a statin;
iii) an mTOR inhibitor and a statin;
iv) an mTOR inhibitor, a TGFβ agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ agonist;
ix) a purinergic receptor antagonist, an mTOR inhibitor, a TGFβ agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

In one embodiment, the at least one agent is selected from the group consisting of:
i) an mTOR inhibitor and a TGFβ agonist;
ii) a statin;
iii) an mTOR inhibitor, a TGFβ agonist, and a statin;
iv) a purinergic receptor antagonist;
v) an agent which disrupts mitochondrial electron transport in the DCs;

In one embodiment, at least one agent comprises an mTOR inhibitor and a TGFβ agonist.

In one embodiment, the mTOR inhibitor comprises rapamycin or a derivative or analog thereof.

In one embodiment, the TGFβ agonist is selected from the group consisting of TGFβ1, TGFβ2, TGFβ3, and mixtures thereof.

In one embodiment, the at least one agent comprises a purinergic receptor antagonist.

In one embodiment, the purinergic receptor antagonist binds to a purinergic receptor selected from the group consisting of P1, P2X, P2X7, and P2Y.

In one embodiment, the purinergic receptor antagonist is oxidized ATP.

In one embodiment, a method of the invention further comprises contacting the induced tolerogenic DCs or the starting population of cells with an antigen to which tolerance is desired.

In one embodiment, the antigen is a crude antigen. In one embodiment, the antigen is a refined antigen. In one embodiment, the antigen comprises one or more of: one or more short peptides; one or more polypeptides; a polypeptide mixture; and one or more proteins. In one embodiment, the antigen comprises a cell lysate or a tissue lysate.

In one embodiment, the antigen comprises one or more peptides, polypeptides, or proteins derived from food. In one embodiment, the antigen comprises one or more peptides, polypeptides, or proteins derived from neural cells or tissue. In one embodiment, the antigen comprises an allergen or a mixture of allergens.

In another aspect, a method of the invention further comprises administering the induced tolerogenic DCs to a subject. In another aspect, a method of the invention further comprises contacting the induced tolerogenic dendritic cells with effector T cells

In another aspect, a method of the invention further comprises testing the ability of the induced tolerogenic DCs to induce Foxp3 expression in naïve T cells prior to the step of administering.

In another aspect, a method of the invention further comprises testing the ability of the induced tolerogenic DCs to delete effector T cells or convert FoxP3⁻ effector T cells to FoxP3⁺ effector T cells prior to the step of administering.

In another aspect, a method of the invention further comprises testing the ability of the cells to retain their tolerogenic phenotype upon stimulation with at least one TLR agonist is tested prior to the step of administering.

In another aspect, a method of the invention further comprises testing the inability of the DCs to increase their oxygen consumption rate upon stimulation with at least one TLR agonist is tested prior to the step of administering.

In one embodiment, the invention pertains to a method of reducing T effector cell responses to an antigen, comprising contacting effector T cells with the composition comprising induced tolerogenic DCs of the invention

In one embodiment, the step of contacting occurs in vivo.

In another aspect, the invention pertains to a method of producing a composition comprising induced immunogenic dendritic cells, the method comprising contacting a starting population of cells comprising dendritic cells or dendritic cell precursors ex vivo with a stimulus which increases oxygen consumption in the dendritic cells, to thereby produce a composition comprising induced immunogenic dendritic cells.

In one embodiment, the starting population comprises fully differentiated dendritic cells.

In another aspect, a method of the invention further comprises measuring the mitochondrial activation of the induced immunogenic dendritic cells.

In one embodiment, the mitochondrial activation of the cells is measured by determining oxygen consumption of the cells upon treatment with at least one TLR agonist.

In another aspect, a method of the invention further comprises contacting the induced immunogenic DCs or the starting population of cells with an antigen to which increased effector T cell response is desired.

In one embodiment, the antigen is derived from a pathogenic organism or toxin.

In one embodiment the antigen is derived from cancer cells.

In one embodiment the antigen is a crude antigen. In one embodiment the antigen is a refined antigen. In one embodiment the antigen comprises one or more of: one or more short peptides; one or more polypeptides; or one or more proteins.

In one embodiment, a method of the invention further comprises administering the cells to a subject.

In one embodiment, a method of the invention further comprises contacting the induced immunogenic dendritic cells with effector T cells.

In one embodiment, the step of contacting occurs in vivo.

In another aspect, the invention pertains to a method of increasing T effector cell responsiveness to an antigen in a subject, comprising contacting a population of dendritic cells with an immunogenic stimulus which increases oxygen consumption in the dendritic cells, wherein the induced immunogenic DCs display an antigen to which increased T effector cell responsiveness is desired on their surface, to thereby increase T effector cell responsiveness to an antigen in the subject.

In one embodiment, a method of the invention further comprises measuring the mitochondrial activation of the cells in response to stimulus with at least one TLR agonist.

In one embodiment, a method of the invention further comprises contacting the induced immunogenic DCs or the starting population of cells are contacted with an antigen to which increased T effector cell responsiveness is desired.

In another aspect, the invention pertains to a method of identifying an agent which promotes antigen-specific tolerance, comprising contacting a population of cells comprising dendritic cells or dendritic cell precursors with a test agent to obtain treated cells, measuring effect of the agent on mitochondrial activation, wherein agents that prevent or reverse mitochondrial activation in the treated cells as compared to an appropriate control are selected as candidate agents for promoting antigen-specific T cell tolerance.

In one embodiment, the effect of the agent on oxygen consumption in the dendritic cells is measured and agents that reduce or do not increase the oxygen consumption rate are selected.

In one embodiment, a method of the invention further comprises testing the ability of the treated cells to convert naïve T cells to Foxp3+ T regulatory cells ex vivo.

In one embodiment, a method of the invention further comprises testing the ability of the treated cells to delete effector T cells ex vivo and/or in vivo.

In one embodiment, a method of the invention further comprises testing the ability of the treated cells to retain their tolerogenic phenotype upon stimulation with at least one TLR agonist ex vivo and/or in vivo.

In another aspect, the invention pertains to a method of identifying an agent which promotes an antigen-specific T effector cell response, comprising contacting a population of cells comprising dendritic cells or dendritic cell precursors with a test agent, measuring the effect of the agent on mitochondrial activation, wherein agents that increase mitochondrial activation as compared to an appropriate control are selected as candidate agents for promoting an antigen-specific T effector T cell response.

In one embodiment, the effect of the agent on oxygen consumption in the dendritic cells is measured and agents that increase the oxygen consumption rate are selected.

In one embodiment, a method of the invention further comprises testing the ability of the treated cells to increase the number or activity of effector T cells ex vivo and/or in vivo.

### BRIEF DESCRIPTION OF THE DRAWINGS

FIG. 1: Differentiation of Foxp3+ Tregs by tolerogenic DCs ex vivo. a) Depiction of procedure used to screen compounds for the capability to induce a tolerogenic phenotype in DCs. b) Percentages of Foxp3 and CD25 expressing cells identified by intracellular staining or GFP expression following 5 days in co-culture with treated DCs. c) Flow cytometry depicting T cells at day five of co-culture with dendritic cells, after staining for the indicated markers. d) Role of TCR signaling strength on Foxp3 induction. Proportion of Foxp3+CD25+ cells among single CD4+CD11c−7AAD− cells at 5 days after co-culture with DC treated as indicated, in the presence of varying concentrations of αCD3. e) Foxp3 expression in T cells at different time points following co-culture with DC in the presence or absence of the cell cycle blocker Aphidicolin (Aph). f) Comparison of the expression of Foxp3 on natural occurring Treg (nTreg) and itDC-induced Treg. g) Comparison of the phenotype of natural occurring Treg (nTreg) and itDC-induced Treg. h) FACS sorting strategy for Foxp3+ and Foxp3− cells for suppression assays. i) Suppression assay. CFSE profile of Tn cells isolated from co-culture with Foxp3+ Treg or Foxp3− Teff and DC. j) Comparison of freshly isolated Foxp3+ nTreg to newly differentiated Treg, at different ratios of suppressor Treg versus responder Tn cells labeled with CFSE.

FIG. 2: Human tolerogenic monocyte-derived dendritic cells (MoDC) induce Foxp3-expressing T cells, a) Cell phenotype: Flow cytometry showing input Tn cells and MoDC, and the Foxp3 and CD25 expression profile of T cells following co-culture with MoDC treated as indicated. b) Compiled data of five independent experiments. P values were calculated using a Bonferroni post-test of a regular two-way ANOVA test. c) Phenotype of MoDC. d) Suppression assay with human Treg. T cells activated by itMoDC that express activation markers (CD45RA−CD25+/−+Teff) and putative Tregs (CD45RA−CD25 high) were cocultures with MoDC and fresh CFSE-labeled Tn in presence of anti-CD3. Histograms show CFSE dilution, a measure of Tn proliferation, after 3-day culture with Teff or putative Treg.

FIG. 3: Phenotype of induced tolerogenic DC. a) Mean fluorescence intensity (MFI) of DC treated with the indicated stimuli, and stained for MHC CLI1, CD80, CD86, or ICOSL. Ctrl: untreated; It: rapamycin/TGFβ; lps: lipopolysaccharides. b) The function of induced tolerogenic DC is toll Like Receptor-agonist resistant. DC were conditioned with rapamycin plus TGFβ and TLR agonists and their effect on Foxp3 induction was measured. c) Sorting strategy for DC subsets. d) Percentage of Foxp3+CD25+ T cells following co-culture with four CD11c+ DC populations. e) Expression and phosphorylation status of different factors in the mammalian target of rapamycin (mTOR) pathway in DC. f) Kinetics of Expression and phosphorylation status of different factors in the mammalian target of rapamycin (mTOR) pathway in DC. g) Comparison of DC of different origins. Bone marrow-derived DC (BMDC) differentiated in presence of GM-CSF or FLT3L were compared to splenic DC (SPDC) for their capacity to induce Foxp3 expression on Tn.

FIG. 4: Mechanisms of tolerance induction by itDC. a) Percentage of Foxp3+CD25+ cells following co-culture with DC treated with rapamycin/TGFβ and, optionally, blocking antibodies to IL-10 (JES5-16E3), IL10R (1B1.3a) or TGFβ (1D11) or isotype controls. In panel b-g) indoleamine 2,3-dioxygenase 1 (IDO1), Epstein-Barr-virus-induced gene 3 (EBI3), FasL, IL-6, TSC1, PDL1 and/or PDL2 sufficient or deficient OVAp-loaded DCs were conditioned as in previous figures and used to stimulate GFP− Tn from Fox3GFP x OT-II animals. Results show the percentage of GFP+CD25+CD4+CD11c−7AAD− cells at day 5 of culture.

FIG. 5: Effects of itDC on T cells ex vivo are contact dependent and influenced by the itDC:T ratio. a) Transwell experiments. OVAp-loaded DC were plated alone or in combination with OTII+ Tn (*) in the upper/lower chamber. The presence of CD25+Foxp3+ among Vα2+CD4+ cells was assessed 5 days later by FACS. b-c) Similar cocultures of itDC and Tn as above were performed but different combinations and ratios of DC were loaded as indicated. The notation 50/20 indicates the ratio of control or LPS treated DC to itDC (i.e., 50:20) and so on. d) The tolerogenic effect of itDC is dependent on the itDC:Tn ratio.

FIG. 6: Depletion of antigen-specific T cells and induction of Foxp3 expression by itDC ex vivo. a) Cellularity of OTII cells in Tn-DC cocultures. OVAp-loaded ctrlDC, itDC and lpsDC, were used to activate OTII+ Tn. Shown are absolute numbers of TCRβ+CD11c−Vα2+7AAD− cells. The broken line represents the input of Tn. b) Normalized data to T cell numbers in cocultures with ctrlDC. White symbols represent results obtained at day 1 whereas filled symbols represent day 2 ratios. c) Tn were left alone (Tn alone), or mixed with different samples of DC as indicated. In one condition, activating αCD3 and αCD28 MAbs were plate-bound to maximally activate Tn in the presence of itDC (itDC+aCD3). In another condition, supplemental IL-2 was added to coculture starting at day 0 (itDC+IL-2). d) itDC reduce Teff cell numbers. Similar experiments as before were performed using Teff cells obtained after activation with lpsDC and subsequently cocultured in presence of all types of DC. Shown are total T cell numbers at the indicated time points. e) Different amounts of DCs were used to stimulate Tn. Shown in the figure are the total numbers of Tn (TCRβ+CD11c−Vα2+7AAD−) at day 1 and 2 after culture with the indicated type of DC at different DC to T cell ratios as indicated (our traditional method consist of the 1:10 DC to Tn ratio).

FIG. 7: Depletion of antigen-specific T cells and induction of Foxp3 expression by itDC in vivo. a) Depiction of the experimental design to test the induction of Foxp3 expression by tolerogenic DC in vivo. b) Homing of DC after intravenous injection. Presence of treated CD45.2+ DC in various organs following transfer via tail vein injection into CD45.1+ recipients. c) Analysis of cells from the popLN at early time points. d) Quantification of the percentage of transferred CD45.2+ cells in the CD4+ Vα2+ fraction. e) Analysis of CD4+Vα2+ cells in the popLN, ingLN and Spl of animals 7 days after injection of DC (late time points). f) Time course and quantification of Foxp3 induction. g) Quantification of the percentage of Foxp3+CD25+ cells among the endogenous CD45.2− cells in the CD4+Vα2+ fraction.

FIG. 8: Effects of itDC administration on the course of a disease: mouse models of autoimmunity, allogenic responses and allergic asthma. a) EAE preventive and therapeutic experimental protocols (top); EAE mean score for different treatment groups (bottom). b) Comparison of DC treatment using the preventive or therapeutic protocols. c) Statistical significance (p value) between EAE treatment groups. d) Compiled data for 5 independent experiments. e) Parameters of EAE for the previous figure. f) Statistical robustness of itDC therapy. g) Percentage and total number of Treg and Teff cells after therapeutic DC injection among CD4+ live single cells. h) Correlation between mean EAE score and Treg/Teff ratio. i) Inhibition by itDC of T cell proliferation in response to allo-Ag in MLR. Balb/c DC (presenters) were mixed with CFSE-labeled C57BL/6 T cells (responders). Differentially treated F1 DC (10⁴/well) were added to some wells. After 4 days, proliferation of responders was analyzed by FACS. C57BL/6 responders alone (without presenters) were used as negative (no stimulation) or positive controls (with anti-CD3 stimulation). j) Protocol of allergic asthma model and DC therapy. k) C57/BL6 sensitized and challenged with OVA were left untreated (ctrl) or injected i.v. different types of DC. BAL fluid was evaluated for eosinophils (CD11b⁺Gr1^{lo}SSC-A^{hi}) and l) CD4⁺ T cells. m) Protocol for HDM sensitization to induce allergic asthma. n) Mice were sensitized to HDM and methacholine-induced AHR was measured on D25 after treatment with DC.

FIG. 9: No effect of direct treatment with rapamycin on the proportions of Foxp3+ T cells in the lymph node in vivo. a) Proportions of Foxp3+ cells among adoptively transferred OTII+CD45.1+ or endogenous polyclonal Vα2+CD4+ T cells are not affected by local injection of OVAp alone (10 ug), rapamycin alone (1 ug) or a combination of both. b) Similar results were obtained when injecting directly activating anti-CD3, LPS or rapamycin.

FIG. 10: Screening of additional compounds to induce tolerogenic DC. a) Percentages of Foxp3 and CD25 expressing cells identified by measuring GFP expression of CD4+CD11c−7AAD− cells after 5 days in coculture with DC treated with various tolerogenic stimuli. b) Phenotype of itDC induced using different agents (MFI=mean fluorescence intensity).

FIG. 11: Effect of DC activation on mitochondria. Panel a) shows mitochondrial respiration (the oxygen consumption rate) in control and LPS-stimulated dendritic cells over time. Bars reflect ratios of OCR in activated:control DC at basal and uncoupled conditions. b) Expression kinetics of PGC1a mRNA after LPS treatment by real-time PCR. c) Representative TEM of mitochondria in immature (4° C.) and LPS-matured (4 h) DC. d) Density of mitochondrial christae was determined in TEM images using ImageJ software as: total cristae length (nm) in a mitochondrium:area (nm²) of the same mitochondrium.

FIG. 12: OCR and immunogenicity of DC. a) Normalized OCR (to the control) in different types of DC after 2 h activation with TLR agonists LPS (TLR4), CpG (TLR9), poly-IC (TLR3) or ATP or not. b) Effect of oATP on OCR in DC. c) Inhibition of electron transport in mitochondria by rotenone blocks DC immunogenicity. CFSE labeled OT-II T cells were incubated with OVA peptide pulsed DC that had been matured with LPS alone or LPS plus 1 mM rotenone. Control DC were treated with LPS without OVA pulsing.

FIG. 13: Induction of tolerogenic function by treatment of DC with statins. DC were treated with Atorvastatin (Atorva, 10 uM), Pravastatin (Prava, 50 uM) or oATP (100 μM) in combination or not with rapamycin, TGFβ and LPS as indicated. These cells were used to activate OTIIxFoxp3GFPxCD45.1 Tn and after 5 days in culture the presence of CD25+Foxp3+ among Vα2+CD4+CD45.1+ cells was assessed by FACS. Positive controls were cell cultures in which additional TGFβ and IL-2 were added (ctrlDC+T2).

### DETAILED DESCRIPTION OF THE INVENTION

Regulation of immune responses is central for the prevention of inflammatory and autoimmune disorders. While downregulation of the immune system can be achieved by way of immunosuppressive therapy, agents that generally suppress the immune system leave subjects susceptible to other disorders, including infections and cancers. A means for controlling the aberrant activation of an immune response to specific antigens would be a major advance in the treatment of autoimmune disorders, as it would allow downregulation of the immune response against a particular target antigen, but would otherwise leave the immune system functional against invading pathogens and tumor associated antigens. Conversely, methods of specifically improving immunogenicity of specific antigens to which immune responses are desired would be of tremendous benefit in promoting desired immune responses, for example in the context of vaccination and promoting responsiveness to tumor antigens.

DCs play a crucial role in stimulating and inhibiting T cell-mediated effector responses. The present invention is based, at least in part, on the discovery of induced tolerogenic DCs which possesses at least one of the following characteristics: i) induced tolerogenic DCs are capable of converting naïve T cells to Foxp3⁺ T regulatory cells ex vivo and in vivo; ii) induced tolerogenic DCs are capable of deleting effector T cells ex vivo and in vivo; iii) induced tolerogenic DCs retain their tolerogenic phenotype upon stimulation with at least one TLR agonist ex vivo (although, in one embodiment, they increase expression of costimulatory molecules in response to such stimulus); and/or iv) induced tolerogenic DCs do not transiently increase their oxygen consumption rate upon stimulation with at least one TLR agonist ex vivo. Induced tolerogenic DCs are characterized by antigen specific tolerance induction ex vivo and in vivo and can be generated ex vivo using, e.g., crude, refined, or cell intrinisic antigen sources.

The invention also provides induced immunogenic dendritic cells, which can be used to stimulate effector T cell responses. Induced immunogenic DCs transiently increase their oxygen consumption rate upon stimulation with at least one TLR agonist ex vivo and, in one embodiment, this can be tested prior to further manipulation of the cells. Induced immunogenic DCs are more immunogenic than uninduced DCs in vivo and ex vivo and can be generated ex vivo using, e.g., crude or refined antigen sources.

### I. Definitions

So that the invention may be more readily understood, certain terms are first defined.

“Dendritic cells (also referred to herein as DC)” are antigen-presenting immune cells that process antigenic material and present it to other cells of the immune system, most notably to T cells. Immature DC function to capture and process protein antigen. When DC endocytose antigens, they process the antigens into smaller peptides that are displayed on the DC surface, where they are presented to antigen-specific T cells. After uptake of antigen, DC migrate to the lymph nodes. Immature dendritic cells are characterized by high endocytic and micropinocytotic function. During maturation, DC can be prompted by various signals, including signaling through Toll-like receptors (TLR), to express co-stimulatory signals that induce cognate effector T cells (Teff) to become activated and to proliferate, thereby initiating a T-cell mediated immune response to the antigen. Alternatively, DC can present antigen to antigen-specific T cells while failing to providing co-stimulatory signals (or while providing co-inhibitory signals), such that Teff are not properly activated. Such presentation can cause, for example, death or anergy of T cells recognizing the antigen, or can induce the generation and/or expansion of regulatory T cells (Treg).

The term “dendritic cells” includes differentiated dendritic cells, whether immature and mature dendritic cells. These cells can be characterized by expression of certain cells surface markers (e.g., CD11c, MHC class II, and at least low levels of CD80 and CD86). In addition, dendritic cells can be characterized functionally by their capacity to stimulate alloresponses and mixed lymphocyte reactions (MLR).

As used herein the term “dendritic cell precursors” includes hematopoietic bone marrow progenitor cells, monocytes, or immature dendritic cells that can mature or can be made to mature into dendritic cells ex vivo.

Starting populations of cells comprising dendritic cells and/or dendritic cell precursors may be “induced” by treatment ex vivo to become tolerogenic or immunogenic. In one embodiment, starting populations of cells are differentiated into dendritic cells prior to, as part of, or after induction, for example using methods known in the art that employ cytokines and/or maturation factors. In one embodiment, induced dendritic cells comprise fully differentiated dendritic cells. In one embodiment, induced dendritic cells comprise both immature and mature dendritic cells. In another embodiment, induced dendritic cells are enriched for mature dendritic cells. In one embodiment, induced dendritic cells express class II molecules on their surface. In another embodiment, induced dendritic cells express class II molecules and costimulatory molecules on their surface.

As used herein, the term “tolerogenic dendritic cell” refers to a dendritic cell capable of suppressing an antigen-specific T cell-mediated immune response, e.g., by reducing effector T cell responses to specific antigens. This can be achieved by, for example, increasing the number of antigen-specific regulatory T cells relative to antigen-specific effector T cells in a T cell population.

As used herein, the term “induced tolerogenic DC” refers to a tolerogenic dendritic cell which is induced ex vivo. Induced tolerogenic dendritic cells have a tolerogenic phenotype that is characterized by at least one of the following properties i) induced tolerogenic DCs are capable of converting naïve T cells to Foxp3⁺ T regulatory cells ex vivo and in vivo; ii) induced tolerogenic DCs are capable of deleting effector T cells ex vivo and in vivo; iii) induced tolerogenic DCs retain their tolerogenic phenotype upon stimulation with at least one TLR agonist ex vivo (and in one embodiment, they increase expression of costimulatory molecules in response to such stimulus); and/or iv) induced tolerogenic DCs do not transiently increase their oxygen consumption rate upon stimulation with at least one TLR agonist ex vivo.

Induced tolerogenic DCs are characterized by antigen specific tolerance induction ex vivo and in vivo. As used herein, the term “antigen specific tolerance induction” means induction of tolerance in effector CD4⁺ T cells to one or more antigens of interest expressed by the induced tolerogenic dendritic cells ex vivo. Such antigens of interest can be expressed by the induced tolerogenic dendritic cells (e.g., as a germline gene product or as the product of an expression vector) or can be contacted with the induced tolerogenic dendiritc cells (or the starting population of dendritic cells or dendritic cell precursors prior to induction) ex vivo.

As used herein, the term “immunogenic dendritic cell” refers to a dendritic cell capable of enhancing an antigen-specific T cell-mediated immune response ex vivo or in vivo, e.g., by increasing effector T cell responses to specific antigens. This can be achieved by, for example, increasing the number of antigen-specific effector T cells in a T cell population and/or by increasing the activity of antigen-specific effector T cells.

As used herein, the term “induced immunogenic dendritic cell” refers to an immunogenic DC which is induced ex vivo. Induced immunogenic dendritic cells are more immunogenic than uninduced DCs and, in one embodiment, have a phenotype that is characterized by a transient increase their oxygen consumption rate upon stimulation with at least one TLR agonist ex vivo.

As used herein, the term “naïve T cell” refers to a T cell that has not previously been stimulated by encounter with antigen. Naïve CD4⁺ T cells can be isolated using methods known in the art, e.g., by enriching for CD4⁺ cells characterized as CD4⁺CD25⁻ CD62L^{high}CD44^{low}Foxp3⁻.

As used herein, the term “regulatory T cell (or T regulatory cell or Treg)” refers to a CD4⁺CD25⁺Foxp3⁺ T cell that negatively regulates the activation of other T cells, including effector T cells. Treg cells are characterized by sustained suppression of effector T cell responses.

As used herein the term “effector T cell or Teff” refers to T cells which are not regulatory and which have encountered antigen and costimulatory molecules. Effector cells can be characterized by certain markers of activation, e.g., cytokine production. In one embodiment, effector T cells are CD4⁺.

As used herein, the term “tolerogenic stimulus” refers to an agent or substance (or combination of agents or substances) that induces a dendritic cell to become tolerogenic, e.g., by inducing the dendritic cell to become capable of increasing the proportion of antigen-specific regulatory T cells to antigen-specific effector T cells in a population. In one embodiment, the increase in antigen-specific regulatory T cells is statistically significantly more than that observed using rapamycin alone.

As used herein the term “agent that promotes respirostatic tolerance” refers to at least one agent that when contacted with a population of cells comprising dendritic cells or dendritic cell precursors ex vivo generates a population of cells comprising induced tolerogenic dendritic cells which do not transiently increase their oxygen consumption rate upon stimulation with at least one TLR agonist ex vivo. The induced tolerogenic dendritic cells induced by these agents are capable of at least one of i) converting naïve T cells to Foxp3⁺ T regulatory cells ex vivo and in vivo; ii) deleting effector T cells ex vivo and in vivo; and iii) increasing expression of costimulatory molecules on the DCs while the DCs retain their tolerogenic phenotype upon stimulation with at least one TLR agonist ex vivo.

As used herein, the term “respirostatic” refers to a protocol or a reagent that does not transiently increase mitochondrial activity of cells as measured in an ex vivo environment; and which inhibits or blocks a subsequent increase in mitochondrial activity in the cells in response to a TLR agonist by the cells. In one embodiment, mitochondrial activity is measured by determining the oxygen consumption of a cell. As used herein, a “respirostatic tolerizing protocol” refers to a protocol under which dendritic cells or dendritic cell precursors are treated in an ex vivo environment to render them capable of at least one of i) converting naïve T cells to Foxp3⁺ T regulatory cells ex vivo and ii) deleting effector T cells ex vivo.

As used herein, the term “respirostimulatory” refers to a protocol or a reagent that transiently increases mitochondrial activity of cells as measured in an ex vivo environment and which does not inhibit or block a subsequent increase in mitochondrial activity in response to a TLR agonist by the cells. In one embodiment, mitochondrial activity is measured by determining the oxygen consumption of a cell.

As used herein, the term “tolerogenically locked” refers to the fact that induced tolerogenic dendritic cells maintain their tolerogenic phenotype even upon stimulus with at least one TLR agonist. A “tolerogenic locking protocol” refers to a protocol in which dendritic cells or dendritic cell precursors are treated in an ex vivo environment with a tolerogenic locking agent which renders them capable of at least one of: i) converting naïve T cells to Foxp3⁺ T regulatory cells ex vivo and ii) deleting effector T cells ex vivo.

As used herein, the term “converting naïve T cells to Foxp3⁺ T regulatory cells” refers to the ability of a population of cells comprising induced tolerogenic dendritic cells to induce expression of the transcription factor Foxp3 in naïve T cells, e.g., in the absence of cell division, such that naïve T cells that did not previously express Foxp3 are induced to express Foxp3 and become T reg cells. In addition, to expression of Foxp3, T regulatory cells (Treg cells) express CD25 and are capable of sustained suppression of effector T cell responses.

As used herein the term “Toll-like receptor or TLR” refers to a family of molecules which serve as pattern recognition receptors for molecules derived from microbes and which stimulate innate immune responses. Agonists of TLRs include, e.g., lipopeptides, glycolipids, lipoproteins, double-stranded RNA, lipopolysaccharide, flagellin, single-stranded RNA, and CpG oligonucleotides, depending upon the TLR.

As used herein, the term “costimulatory molecules” which can be expressed by dendritic cells refers to costimulatory molecules such as CD80, CD86, and ICOS ligand. In one embodiment, these molecules are expressed by induced tolerogenic DCs, yet the cells maintain their tolerogenic phenotype.

As used herein, the term “class II molecules” refers to polymorphic, heterodimeric membrane proteins found on the surface of antigen-presenting cells. Class II molecules bind and display peptide fragments of protein antigens which are recognized by T lymphocytes. Class II molecules usually display peptides derived from extracellular proteins (e.g., exogenous proteins not made by the cell expressing the class II molecules) which are internalized into phagocytic or endocytic vesicles and processed, or which can bind to the peptide binding cleft of MHC class II when directly contacted with cells.

As used herein, the term “crude antigen” refers to an antigen which is not molecularly characterized. For example, a crude antigen preparation may comprise a cell lysate or a tissue lysate. In another embodiment, a crude antigen preparation may comprise an extract of a protein or mixture of proteins (e.g., an allergen or mixture of allergens).

As used herein, the term “intrinsic antigen” refers to a polypeptide or peptide made by a cell (e.g., as the product of a germline gene, self protein, or self peptide) which is presented on the surface of the cell in a form that can be recognized by T cells, e.g., i) a class I molecule or ii) a peptide associated with class II molecules or, more commonly, class I molecules.

As used herein, the term “refined antigen” refers to an antigen which has been at least partially purified. In one embodiment, such an antigen has been purified to remove unwanted material. In another embodiment, a refined antigen is an antigen that has been molecularly characterized. For example, in one embodiment, a refined antigen can comprise a defined peptide or mixture of such peptides.

### II. Starting Populations of Cells

To obtain starting cell populations which comprise dendritic cell precursors and/or dendritic cells, samples of cells, tissues, or organs comprising dendritic cell precursors or dendritic cells are isolated from one or more subjects using methods known in the art. Such starting cell populations may be obtained from one subject or may be pooled from more than one donor.

In one embodiment, a starting population which comprises dendritic cells and/or dendritic cell precursors is derived from splenic tissue. In one embodiment, a starting cell population which comprises dendritic cells and/or dendritic cell precursors is derived from thymic tissue. In one embodiment, a starting cell population which comprises dendritic cells and/or dendritic cell precursors is derived from bone marrow. In one embodiment, a starting cell population which comprises dendritic cells and/or dendritic cell precursors is derived from peripheral blood, e.g., from whole blood or using leukophoresis.

In one embodiment, a starting cell population of cells comprises dendritic cell precursors. In one embodiment, a population of cells comprising dendritic cell precursors can be harvested from the peripheral blood using standard mononuclear cell leukopheresis, a technique that is well known in the art. Dendritic cell precursors can then be collected, e.g., using sequential buoyant density centrifugation steps. For example, the leukopheresis product can be layered over a buoyant density solution (specific gravity=1.077 g/mL) and centrifuged at 1,000 g for 20 minutes to deplete erythrocytes and granulocytes. The interface cells are collected, washed, layered over a second buoyant density solution (specific gravity=1.065 g/mL), and centrifuged at 805 g for 30 minutes to deplete platelets and low-density monocytes and lymphocytes. The resulting cell pellet is enriched for dendritic cell precursors.

In another embodiment, a starting population of cells comprising dendritic cells can be obtained using methods known in the art. Such a population may comprise myeloid dendritic cells (mDC), plasmacytoid dendritic cells (pDC), and/or dendritic cells generated in culture from monocytes (e.g., MO-DC, MDDC).

In one embodiment, dendritic cells and/or dendritic cell precursors can also be derived from a mixed cell population containing such cells (e.g., from the circulation or from tissue or an organ). In certain embodiments, the mixed cell population containing DC and/or dendritic cell precursors is enriched such that DC and/or dendritic cell precursors make up greater than 50% (e.g., 55%, 60%, 65%, 70%, 75%, 80%, 85%, 90%, 95%, 98%, 99%, 99.5%, 99.9% or more) of the cell population. In some embodiments, the dendritic cells described herein are purified by separation from some or all non-dendritic cells in a cell population. In exemplary embodiments, cells can be purified such that a starting population comprising dendritic cells and/or dendritic cell precursors contains at least 50% or more dendritic cells and/or dendritic cell precursors, e.g., a purity of 50%, 55%, 60%, 65%, 70%, 75%, 80%, 85%, 90%, 95%, 98%, 99%, 99.5%, 99.9% or more.

In one embodiment, dendritic cells can be isolated using the techniques described in Current Protocols in Immunology, Wiley Interscience, Nov. 19, 2009, or in Woo et al., Transplantation, 58:484 (1994), the entire contents of which are incorporated herein by reference. Those skilled in the art are able to implement modifications to the foregoing methods of isolating cells comprising dendritic cells and/or dendritic cell precursors without the exercise of undue experimentation. In one embodiment, dendritic cells can be purified using fluorescence-activated cell sorting for antigens present on their surface, e.g., CD11c in the case of certain dendritic cells. In one embodiment, DCs present in a starting population of cells express CD11c. In another embodiment, DCs and/or dendritic cell precursors present in a starting population of cells express class II molecules. A starting population of cells may be monitored for expression of various cell surface markers (e.g., including CD11c) using techniques known in the art.

In another embodiment, a population of cells comprising dendritic cells and/or dendritic cell precursors can be obtained from pluripotential cells present in blood as PBMCs. Although most easily obtainable from blood, the pluripotential cells may also be obtained from any tissue in which they reside, including bone marrow and spleen tissue. These pluripotential cells typically express CD14, CD32, CD68 and CD115 monocyte markers with little or no expression of CD83, p55 or accessory molecules such as CD40 and CD86.

In one embodiment, dendritic cell precursors can be differentiated into dendritic cells using methods known in the art prior to, during, or after treatment with at least one agent in a protocol to prepare induced tolerogenic or induced immunogenic dendritic cells. For example, when cultured in the presence of cytokines such as a combination of GM-CSF and IL-4 or IL-13, the pluripotential cells give rise to the immature dendritic cells. In another embodiment, FLT3 Ligand can be used for this purpose. For example, in one embodiment, a starting population of cells comprising dendritic cells and/or dendritic cell precursors can be cultured ex vivo in the presence of one or more agents which promote differentiation of DCs. In one embodiment, one or more of GMCSF or IL-4 is used to promote the development of DCs ex vivo, e.g., by culture for 1-15 days, 2-10 days, 3-9 days, 4-8 days, or 5-6 days or such other time to obtain sufficient differentiation. In one embodiment, induced dendritic cells are fully differentiated (either prior to, during, or after induction to produce induced tolerogenic dendritic cells or induced immunogenic dendritic cells.)

In another embodiment, a starting population of cells comprising DCs and/or DC precursors can be obtained from PBMCs. Methods of obtaining PBMCs from blood, using methods such as differential sedimentation through an appropriate medium, e.g. Ficoll-Hypaque [Pharmacia Biotech, Uppsala, Sweden], are well known and suitable for use in this invention. In a preferred embodiment of the invention, the pluripotential cells are obtained by depleting populations of PBMCs of platelets, and T and B lymphocytes. Various methods may be used to accomplish the depletion of the non-pluripotential cells. According to one method, immunomagnetic beads labeled with antibodies specific for cells to be removed, e.g., T and/or B lymphocytes, either directly or indirectly may be used to remove the T and B cells from the PBMC population. T cells may also be depleted from the PBMC population by rosetting with neuramimidase treated red blood cells as described by O'Dherty (1993), which is incorporated herein by reference.

In one embodiment, to produce 3 million mature dendritic cells, approximately 40 mls of blood can be processed. In one embodiment, 4 to 8×10⁷ pluripotential PBMC give rise to approximately 3 million mature dendritic cells.

As set forth above, cultures of immature dendritic cells may be obtained by culturing the pluripotential cells in the presence of cytokines which promote their differentiation for a time sufficient to achieve the desired level of differentiation, e.g., from 1-10 days, from 2-9 days, from 3-8 days, or from 4-7 days. As an example, a combination of GM-CSF and IL-4 at a concentration of each at between about 200 to about 2000 U/ml, between about 500 and 1000 U/ml, or about 800 U/ml (GM-CSF) and 1000 U/ml (IL-4) produces significant quantities of the immature dendritic cells. A combination of GM-CSF (10-200 ng/ml) and IL-4 (5-50 ng/ml) can also be used. It may also be desirable to vary the concentration of cytokines at different stages of the culture such that freshly cultured cells are cultured in the presence of higher concentrations of IL-4 (1000 U/ml) than established cultures (500 U/ml IL-4 after 2 days in culture). Other cytokines such as IL-13 may be found to substitute for IL-4. In another embodiment, FLT3 ligand can be used for this purpose. Other protocols for this purpose are known in the art.

Methods for obtaining these immature dendritic cells from adherent blood mononuclear fractions are described in Romani et al. (1994); and Sallusto and Lanzavecchia, 1994) both of which are incorporated herein by reference. Briefly, lymphocyte depleted PBMCs are plated in tissue culture plates at a density of about 1 million cells/cm² in complete culture medium containing cytokines such as GM-CSF and IL-4 at concentrations of each at between about 800 to 1000 U/ml and IL-4 is present at about 1000 U/ml.

Another source of immature dendritic cells is cultures of proliferating dendritic cell precursors prepared according to the method described in Steinman et al. International application PCT/US93/03141, which is incorporated herein by reference. Since the dendritic cells prepared from the CD34⁺ proliferating precursors mature to dendritic cells expressing mature characteristics it is likely that they also pass through a development stage where they are pluripotential.

In one embodiment, a starting population of cells comprising dendritic cells can be enriched for the presence of mature dendritic cells by contacting the immature dendritic cells with a dendritic cell maturation factor. As referred to herein, the dendritic cell maturation factor may actually be one or more specific substances which act alone or with another agent to cause the maturation of the immature dendritic cells, for example, with one or more of an adjuvant, a TLR agonist, a CD40 agonist, an inflammasome activator, an inflammatory cytokine, or combinations thereof.

### III. Induced Tolerogenic Dendritic Cells

In one embodiment, the starting population of cells comprising dendritic cells and/or dendritic cell precursors is stimulated ex vivo with one or more agents that produce induced tolerogenic DCs in the population. Induced tolerogenic DCs are capable of suppressing a T cell-mediated immune response to the antigen presented by the DC by, for example, increasing the proportion of antigen-specific Treg cells relative to antigen-specific effector T cells in a cell population. This increase in the proportion of antigen-specific Treg cells can be brought about in a number of ways. More specifically, induced tolerogenic dendritic cells have a tolerogenic phenotype that is characterized by at least one of the following properties i) induced tolerogenic DCs are capable of converting naïve T cells to Foxp3⁺ T regulatory cells ex vivo and in vivo; ii) induced tolerogenic DCs are capable of deleting effector T cells ex vivo and in vivo; iii) induced tolerogenic DCs retain their tolerogenic phenotype upon stimulation with at least one TLR agonist ex vivo (and in one embodiment, increase expression of costimulatory molecules with the same stimulus); and/or iv) induced tolerogenic DCs do not transiently increase their oxygen consumption rate upon stimulation with at least one TLR agonist ex vivo. In one embodiment, induced tolerogenic DCs have at least 2 of the above properties. In another embodiment, induced tolerogenic DCs have at least 3 of the above properties. In yet another embodiment, induced tolerogenic DCs have all 4 of the above properties.

Conversion of Naïve T Cells to Foxp3⁺ Treg Cells

In some embodiments, tolerogenic dendritic cells are capable of inducing Foxp3 expression and/or CD25 expression in naïve T cells (e.g., CD4⁺CD25⁻ T cells) ex vivo, thereby producing Tregs. Tolerogenic DC capable of inducing Foxp3 expression in naïve T cells can induce Foxp3 expression in the presence or in the absence of exogenous cytokines. For example, in some embodiments, the differentiation of Treg cells is IL-10 and/or TGFβ independent. In one embodiment, the induction of Foxp3 by induced tolerogenic DCs requires cell-cell contact.

The ability of induced tolerogenic dendritic cells to convert naïve T cells to Foxp3⁺ cells can be measured ex vivo using methods known in the art. For example, naïve T cells can be obtained from the same subject from whom dendritic cells and/or dendritic cell precursors have been obtained. The naïve T cells can be cocultured with induced tolerogenic dendritic cells in the presence of an agent that induces T cell receptor-mediated stimulation (e.g., a pan-T cell stimulatory agent such as anti-CD3 or a superantigen) for a sufficient number of days (e.g, from 1-10 days, from 2-8 days, or from 3-6 days). The cells in the culture can be stained for intracellular Foxp3 expression using antibodies (such as those commercially available from Biolegend™) and the number of cells expressing Foxp3 can be quantitated and compared to an appropriate control. In another embodiment, in lieu of a pan-T cell stimulatory agent, the induced tolerogenic DCs can be contacted with a universal T cell antigen or universal T cell epitope, recognized by cells from individuals with many different HLA-DR and -DQ haplotypes (e.g., as have been described in the art. Such universal antigens have been described, for example, in pathogens, see, e.g., Greenstein et al. 1992 J. Immunol. 148:3970). Various ratios of itDC to Tn can be used to induce Treg differentiation ex vivo. For example, itDC can be co-cultured with Tn at a ratio of 1:100, 1:10, 1:1, 10:1, or 100:1, and ranges therein. In exemplary embodiments, itDC are co-cultured with Tn at a ratio of 1 DC:10 Tn.

In one embodiment, induced tolerogenic DCs are capable of converting naïve T cells to Foxp3⁺ T reg cells in the absence of cell division by the T cells. In one embodiment, such conversion of the input naïve T cells to Foxp3 expressing cells, the assay can be done in the presence of an agent which prevents proliferation, e.g., an inhibitor of the cell cycle or a strong TCR agonist such as an activating anti-CD3 antibody.

In addition to conversion of naïve T cells to Foxp3 expressing Treg cells, in some embodiments, induced tolerogenic DC are also capable of inducing expansion and/or proliferation of Foxp3⁺CD25⁺ Treg cells, ex vivo. This can be demonstrated using methods known in the art, for example by measuring numbers of Foxp3⁺ cells under culture conditions in which proliferation of cells is not prevented and finding that the number of Foxp3+ cells has increased as compared to an appropriate control.

In one embodiment, induced tolerogenic DC described herein are capable of inducing sustained Foxp3 expression in naïve T cells. For example, in some embodiments, the induced tolerogenic DC are capable of inducing Foxp3 expression in naïve T cells that is sustained for a period of 10 or more days, e.g., for at least 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days or more.

In some embodiments, induced tolerogenic DC are also capable of inducing Foxp3 expression in CD4⁺ effector T cells, thereby converting CD4⁺ effector T cells to Tregs. Such effector T cells can include T cells producing Th17 type cytokines, T cells producing Th1 type cytokines, T cells producing Th2 type cytokines, and mixtures thereof.

### Deletion of Effector T Cells

In some embodiments, induced tolerogenic DC are capable of inducing cell death (i.e., deletion) or loss of effector function in effector T cells recognizing the antigen presented by the induced tolerogenic DC. This capability allows induced tolerogenic DC to suppress or reduce the immune response by directly deleting antigen-specific effector T cells. Deletion of effector T cells can be demonstrated using methods known in the art. For example, as set forth above, naïve T cells can be obtained from the same subject from whom dendritic cells and/or dendritic cell precursors have been obtained. The naïve T cells can be cocultured with induced tolerogenic dendritic cells in the presence of an agent that induces T cell receptor-mediated stimulation (e.g., a pan-T cell stimulatory agent such as anti-CD3 or a superantigen) for a sufficient number of days (e.g, from 1-10 days, from 2-8 days, or from 3-6 days). Various ratios of itDC to Tn can be used to induce Treg differentiation ex vivo. For example, itDC can be co-cultured with Tn at a ratio of 1:100, 1:10, 1:1, 10:1, or 100:1, and ranges therein. In exemplary embodiments, itDC are co-cultured with Tn at a ratio of 1 DC:10 Tn. Numbers of T cells can be quantitated during days 1 and 2 in the cultures and compared to an appropriate control. In another embodiment, in lieu of a pan-T cell stimulatory agent, the induced tolerogenic DCs can be contacted with a universal T cell antigen or universal T cell epitope. Numbers of effector T cells can be quantitated, e.g., at days 1 and/or 2 to determine whether there is a decrease in cell number.

In one embodiment, induced tolerogenic DCs are capable of deleting T effector cells ex vivo by day 1 of culture. In one embodiment, induced tolerogenic DCs are capable of deleting T effector cells ex vivo by day 2 of culture. In another embodiment, induced tolerogenic DCs are capable of deleting greater than 10% of T effector cells ex vivo as compared to an appropriate control. In another embodiment, induced tolerogenic DCs are capable of deleting greater than 20% of T effector cells ex vivo as compared to an appropriate control. In another embodiment, induced tolerogenic DCs are capable of deleting greater than 30% of T effector cells ex vivo as compared to an appropriate control. In another embodiment, induced tolerogenic DCs are capable of deleting greater than 40% of T effector cells ex vivo as compared to an appropriate control. In another embodiment, induced tolerogenic DCs are capable of deleting greater than 50% of T effector cells ex vivo as compared to an appropriate control. In another embodiment, induced tolerogenic DCs are capable of deleting greater than 60% of T effector cells ex vivo as compared to an appropriate control.

In one embodiment, the effector T cells are deleted by a mechanism that does not involve apoptosis.

### Tolerogenic Locking

Stimulation of Toll-like receptors (TLR) on the surface of DC promotes DC activation, allowing DC to induce proliferation of effector T cells. Surprisingly, the induced tolerogenic dendritic cells described herein maintain their tolerogenic phenotype (are tolerogenicly locked) even after being contacted with a maturation stimulus ex vivo, e.g., after stimulation with at least one TLR agonist. The presence of the tolerogenic phenotype of the cells can be demonstrated functionally, e.g., by confirming that cells treated with a maturation stimulus retain their functional tolerogenic phenotype as described herein.

In one embodiment, induced tolerogenic dendritic cells treated with a maturation stimulus increase expression of costimulatory molecules (as compared to the level of expression of costimulatory molecules prior to stimulation), but retain their tolerogenic phenotype. Exemplary such costimulatory molecules include one or more of CD80, CD86, and ICOS ligand. In another embodiment, induced tolerogenic dendritic cells treated with a maturation stimulus increase their expression of class II molecules (as compared to the level of expression of class II molecules prior to stimulation), but retain their tolerogenic phenotype.

Exemplary tolerogenic locking agents are set forth in more detail below. In one embodiment, at least one such agent can be used in a tolerogenic locking protocol. For example, in one embodiment, the at least one agent comprises an mTOR inhibitor and a TGFβ agonist. In another embodiment, the at least one agent comprises a statin. In another embodiment, the at least one agent comprises an mTOR inhibitor and a statin. In another embodiment, the at least one agent comprises an mTOR inhibitor, a TGFβ agonist, and a statin. In another embodiment, the at least one agent comprises a purinergic receptor antagonist. In another embodiment, the at least one agent comprises a purinergic receptor antagonist and a statin. In another embodiment, the at least one agent comprises a purinergic receptor antagonist and an mTOR inhibitor. In another embodiment, the at least one agent comprises a purinergic receptor antagonist, an mTOR inhibitor and a TGFβ agonist. In another embodiment, the at least one agent comprises a purinergic receptor antagonist, an mTOR inhibitor, a TGFβ agonist and a statin. In another embodiment, the at least one agent comprises an agent which disrupts mitochondrial electron transport in the DCs. In another embodiment, the at least one agent comprises an agent which disrupts mitochondrial electron transport in the DCs and an mTOR inhibitor. In another embodiment, the at least one agent comprises an agent which disrupts mitochondrial electron transport in the DCs and a statin. In another embodiment, the at least one agent comprises an agent which disrupts mitochondrial electron transport in the DCs, an mTOR inhibitor, and a TGFβ agonist. In another embodiment, the at least one agent comprises an agent which disrupts mitochondrial electron transport in the DCs, an mTOR inhibitor, a TGFβ agonist, and a statin. In one embodiment, a tolerogenic locking agent does not consist of rapamycin alone. In another embodiment, a tolerogenic locking agent does not consist of an mTOR inhibitor alone.

### Respirostasis

The induced tolerogenic DCs of the instant invention are respirostatic, i.e., their mitochondrial activity does not transiently increase upon stimulation with at least one TLR agonist ex vivo. TLR agonists function to increase mitochondrial activation in DCs. Surprisingly, induced tolerogenic dendritic cells do not exhibit this transient increase in mitochondrial activation upon stimulation with one or more TLR agonists.

In one embodiment, oxygen consumption can be used as a readout of mitochondrial activation. The oxygen consumption rate (OCR) can be measured using methods known in the art, e.g., using an analyzer such as the Seahorse XF24 flux analyzer or using a Clark-type electrode.

In another embodiment, alternative readouts of mitochondrial activation can be measured. For example, glucose uptake (e.g., using derivatized glucose) or the presence of reactive oxygen species (e.g., using DCF-DA) can be measured. In another embodiment, lactic acid production (which is elevated with increased glycolysis and/or decreased mitochondrial activity) can be measured. In one embodiment, the extracellular acidification rate (ECAR) can be measured and is reflective of lactic acid production by glycolysis or pyruvate overload. The Seahorse SF24 flux analyzer can be used for this purpose. In yet another embodiment, cellular ATP/ADP ratios may be measured (e.g., using commercially available kits or as in Nagel et al. 2010. Methods Mol. Biol. 645:123-31). Increased levels of ATP and decreased levels of ADP have been recognized in proliferating cells and are a measure of activation.

In another embodiment, the level of expression of a gene which is a marker of mitochondrial activation can be measured. For example, in one embodiment, mRNA levels of the expression of one or more of PGC-1a, PGC-1b, PRC, or other molecules involved in mitochondrial function, such as estrogen-related receptor α, NRF-1, NRF-2, Sp1, YY1, CREB and MEF-2/E-box factors can be measured. In one embodiment, the regulatory region of such a gene or a portion thereof may be operably linked to a reporter gene to facilitate measurement.

As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described, e.g., in, Molecular Cloning: A Laboratory Manual, Third Edition CSHL Press (2001). It should be understood that the design of the expression vector may depend on such factors as the choice of the host cell to be transfected and/or the type and/or amount of protein desired to be expressed. Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

A variety of reporter genes are known in the art and are suitable for use in the screening assays of the invention. Examples of suitable reporter genes include those which encode chloramphenicol acetyltransferase, beta-galactosidase, alkaline phosphatase, green fluorescent protein (and its various mutant forms), or luciferase. Standard methods for measuring the activity of these gene products are known in the art.

Exemplary respirostatic agents are set forth in more detail below. In one embodiment, at least one such agent can be used in a respirostatic protocol. For example, in one embodiment, the at least one agent comprises an mTOR inhibitor and a TGFβ agonist. In another embodiment, the at least one agent comprises a statin. In another embodiment, the at least one agent comprises an mTOR inhibitor and a statin. In another embodiment, the at least one agent comprises an mTOR inhibitor, a TGFβ agonist, and a statin. In another embodiment, the at least one agent comprises a purinergic receptor antagonist. In another embodiment, the at least one agent comprises a purinergic receptor antagonist and a statin. In another embodiment, the at least one agent comprises a purinergic receptor antagonist and an mTOR inhibitor. In another embodiment, the at least one agent comprises a purinergic receptor antagonist, an mTOR inhibitor and a TGFβ agonist. In another embodiment, the at least one agent comprises a purinergic receptor antagonist, an mTOR inhibitor, a TGFβ agonist and a statin. In another embodiment, the at least one agent comprises an agent which disrupts mitochondrial electron transport in the DCs. In another embodiment, the at least one agent comprises an agent which disrupts mitochondrial electron transport in the DCs and an mTOR inhibitor. In another embodiment, the at least one agent comprises an agent which disrupts mitochondrial electron transport in the DCs and a statin. In another embodiment, the at least one agent comprises an agent which disrupts mitochondrial electron transport in the DCs, an mTOR inhibitor, and a TGFβ agonist. In another embodiment, the at least one agent comprises an agent which disrupts mitochondrial electron transport in the DCs, an mTOR inhibitor, a TGFβ agonist, and a statin. In one embodiment, a respirostatic agent does not consist of rapamycin alone. In another embodiment, a respirostatic agent does not consist of an mTOR inhibitor alone.

### IV. Ex Vivo Production of Induced Tolerogenic Dendritic Cells

Induced tolerogenic DCs of the invention are produced ex vivo by contacting starting populations of cells comprising dendritic cells and/or dendritic cell precursors with at least one tolerogenic stimulus, e.g., a respirostatic agent or a tolerogenic locking agent.

Tolerogenic Stimuli

The term “tolerogenic stimuli” as used herein includes substances which, alone or in combination, induce a dendritic cell to become tolerogenic, e.g., by inducing the dendritic cell to become capable of increasing the proportion of antigen specific Treg cells to antigen specific Teff cells in a cell population. More specifically, induced tolerogenic dendritic cells are produced by one or more agents which induce a tolerogenic phenotype in the DCs characterized by at least one of the following properties i) induced tolerogenic DCs are capable of converting naïve T cells to Foxp3+T regulatory cells ex vivo and in vivo; ii) induced tolerogenic DCs are capable of deleting effector T cells ex vivo and in vivo; iii) induced tolerogenic DCs retain their tolerogenic phenotype upon stimulation with at least one TLR agonist ex vivo (while in one embodiment, they increase expression of costimulatory molecules); and/or iv) induced tolerogenic DCs do not transiently increase their oxygen consumption rate upon stimulation with at least one TLR agonist ex vivo.

Exemplary tolerogenic stimuli include those agents which do not increase mitochondrial activation (e.g., as measured by oxygen consumption) or which disrupt electron transport in cells. Other exemplary tolerogenic stimuli include those agents which tolerogenicly lock induced DCs into a tolerogenic phenotype. Exemplary toloergenic stimuli include agents include inhibitors of mammalian Target of Rapamycin (mTOR), agonists of TGFβ pathway signaling, statins, purinergic receptor pathway antagonists, and agents which inhibit mitochondrial electron transport, either alone or in combination. In one embodiment, a tolerogenic stimulus does not consist of rapamycin alone. In another embodiment, a tolerogenic stimulus does not consist of an mTOR inhibitor alone.

In one embodiment, after treatment with one or more tolerogenic stimuli (such as those set forth below, known in the art, or identified using the methods described herein) the cells may be removed from the agents, e.g., by centrifugation and/or by washing prior to further manipulation. Exemplary agents are described in more detail below.

1. mTOR Inhibitors

In an exemplary embodiment, a tolerogenic stimulus for use in the instant invention comprises or consists of an mTOR inhibitor. mTOR inhibitors suitable for practicing the invention include inhibitors or antagonists of mTOR or mTOR-induced signaling. mTOR inhibitors include rapamycin and analogs, portions, or derivatives thereof, e.g., Temsirolimus (CCI-779), everoliums (RAD001) and deforolimus (AP23573). Additional rapamycin derivatives include 42- and/or 31-esters and ethers of rapamycin, which are disclosed in the following patents, all hereby incorporated by reference in their entirety: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers are disclosed in the patents listed above. 27-esters and ethers of rapamycin are disclosed in U.S. Pat. No. 5,256,790, which is hereby incorporated by reference in its entirety. Oximes, hydrazones, and hydroxylamines of rapamycin are disclosed in U.S. Pat. Nos. 5,373,014, 5,378,836, 5,023,264, and 5,563,145, which are hereby incorporated by reference in their entirety. The preparation of these oximes, hydrazones, and hydroxylamines are disclosed in the foregoing patents. The preparation of 42-oxorapamycin is disclosed in U.S. Pat. No. 5,023,263, which is hereby incorporated by reference in its entirety.

Other mTOR inhibitors include PI-103, XL765, Torin1, PP242, PP30, NVP-BEZ235, and OSI-027. Additional mTOR inhibitors include LY294002 and wortmannin. Other inhibitors of mTOR are described in U.S. Pat. Nos. 7,504,397 and 7,659,274, and in Patent Publication Nos. US20090304692A1; US20090099174A1, US20060199803A1, WO2008148074A3, the entire contents of which are incorporated herein by reference.

In one embodiment, an mTOR inhibitor (e.g., rapamycin or a variant or derivative thereof) is used in combination with one or more statins. In one embodiment, an mTOR inhibitor (e.g., rapamycin or a variant or derivative thereof) is used in combination with a TGFβ pathway agonist.

2. TGFβ Pathway Agonists

In an exemplary embodiment, a tolerogenic stimulus for use in the instant invention comprises or consists of one or more TGFβ agonists. TGFβ agonists suitable for practicing the invention include substances that stimulate or potentate responses induced by TGFβ signaling. In one embodiment, a TGFβ pathway agonist is acts by modulating TGFβ receptor-mediated signaling. In another embodiment, a TGFβ pathway agonist is a TGFβ mimetic, e.g., a small molecule having TGFβ-like activity (e.g., biaryl hydroxamates, A-161906 as described in Glaser et al. 2002. Molecular Cancer Therapeutics 1:759-768, or other histone deacetylase inhibitors (such as spiruchostatins A and B or diheteropeptin).

In exemplary embodiments, a TGFβ receptor agonist useful for practicing the invention is TGFβ, including TGFβ1, TGFβ2, TGFβ3, variants thereof, and mixtures thereof. Additional TGFβ agonists are described in Patent Publication No. US20090143394A1, the entire contents of which are incorporated herein by reference.

In particular embodiments, the foregoing TGFβ agonists are used in the presence of an mTOR inhibitor for producing induced tolerogenic DC.

3. Statins

Statins are HMG-CoA reductase inhibitors, a class of drug used to lower cholesterol levels by inhibiting the enzyme HMG-CoA reductase, which plays a central role in the production of cholesterol in the liver. Exemplary statins include atorvastatin (Lipitor and Torvast), fluvastatin (Lescol), lovastatin (Mevacor, Altocor, Altoprev), pitavastatin (Livalo, Pitava), pravastatin (Pravachol, Selektine, Lipostat), rosuvastatin (Crestor), simvastatin (Zocor, Lipex). In one embodiment, at least one statin is used alone for producing induced tolerogenic dendritic cells. In another embodiment, at least one statin is used in combination with an mTOR inhibitor.

4. Purinergic Receptor Pathway Antagonists

In an exemplary embodiment, a tolerogenic stimulus for use in the instant invention comprises or consists of one or more purinergic agonists. Purinergic receptor pathway antagonists suitable for practicing the invention include inhibitors or antagonists of purinergic receptor activity or purinergic receptor signaling. Particular purinergic receptor antagonists include compounds that inhibit the activity of or signaling through the purinergic receptors P1, P2X, P2X7, and/or P2Y. These receptors bind extracellular adenosine triphosphate (ATP). In one embodiment, a purinergic receptor antagonist useful for practicing the invention is oxidized ATP (oATP).

In other embodiments, purinergic receptor antagonists useful for practicing the invention include one or more of the compounds described in the following U.S. patents, the entire contents of which are incorporated herein by reference: U.S. Pat. No. 7,235,549, U.S. Pat. No. 7,214,677, U.S. Pat. No. 7,553,972, U.S. Pat. No. 7,241,776, U.S. Pat. No. 7,186,742, U.S. Pat. No. 7,176,202, U.S. Pat. No. 6,974,812, U.S. Pat. No. 7,071,223, and U.S. Pat. No. 7,407,956. In other embodiments, purinergic receptor antagonists useful for practicing the invention include one or more of the compounds described in the following patent publications, the entire contents of which are incorporated herein by reference: WO2010018280A1, WO2008142194A1, WO2009074519A1, WO2008138876A1, WO2008119825A3, WO2008119825A2, WO2008125600A3, WO2008125600A2, WO06083214A1, WO03047515A3, WO03047515A2, WO03042191A1, WO2008119685A3, WO2008119685A2, WO06003517A1, WO04105798A1, WO2008116814A1, WO2007056046A1, WO2009132000A1, WO2009077559A3, WO2009077559A2, WO2009074518A1, WO2008003697A 1, WO2007056091A3, WO2007056091A2, WO06136004A1, WO05111003A1, WO05019182A1, WO04105796A1, WO04073704A1, WO2009077362A1, US20070032465A1, WO2009053459A1, US20080009541A1, WO2007008157A1, WO2007008155A1, US20070105842A1, WO06017406A1, US20060058302A1, US20060018904A1, WO05025571A1, WO04105797A1, WO04099146A1, WO04058731A1, WO04058270A1, US20030186981A1, WO2009057827A1, US20080171733A1, WO2007002139C1, WO2007115192A3, WO2007115192A2, WO2007002139A3, WO2007002139A2, US20070259920A1, US20070049584A1, WO06086229A1, US20060247257A1, US20060052374A1, WO05014555A1, US20090220516A1, US20090042886A1, US20080207577A1, US20070281939A1, US20070281931A1, US20070249666A1, US20070232686A1, US20070142329A1, US20070122849A1, US20070082930A1, US20070010497A1, US20060217430A1, US20060211739A1, US20060040939A1, US20060025614A1, US20050009900A1, and US20040180894A1.

In particular embodiments, purinergic receptor antagonists useful for practicing the invention include one or more of oATP, suranim, clopidogrel, prasugrel, ticlopidine, ticagrelor, A740003, A438079, pyridoxalphosphate-6-azophenyl-2′,4′-disulfonic acid (PPADS), pyridoxal 5′-phosphate (P5P), periodate-oxidized ATP, 5-(N,N-hexamethylene)amiloride (HMA), KN62 (1-[N,O-bis(5-isoquinolinesulfonyl)-N-methyl-L-tyrosyl]-4-phenylpiperazine), suramin, 2.Chloro-5-[[2-(2-hydroxy-ethylamino)-ethylamino]-methyl]-N-(tricyclo[3.3.1.1^{3,7}]dec-1-ylmethyl)-benzamide, 2.Chloro-5-[3-[(3-hydroxypropyl)amino]propyl]-N-(tricyclo[3.3.1.1]dec-1-ylmethyl)-benzamide, (R)-2-Chloro-5-[3-[(2-hydroxy-1-methylethyl)amino]propyl]-N-(tricyclo[3.3.1.1^{3,7}]dec-1-ylmethyl)-benzamide, 2.Chloro-5-[[2-[(2-hydroxyethyl)amino]ethoxy]methyl]-N-(tricyclo[3.3.1.1^{3,7}]dec-1-ylmethyl)-benzamide, 2.Chloro-5-[3-[3-(methylamino)propoxy]propyl]-N-(tricyclo[3.3.1.1^{3,7}]dec-1-ylmethyl)benzamide, 2.Chloro-5-[3-(3-hydroxy-propylamino)-propoxy]-N-(tricyclo[3.3.1.1^{3,7}]dec-1-ylmethyl)-benzamide, 2.Chloro-5-[2-(3-hydroxypropylamino)ethylamino]-N-(tricyclo[3.3.1.1^{3,7}]dec-1-ylmethyl)-benzamide, 2.Chloro-5-[2-(3-hydroxypropylsulfonyl)ethoxy]-N-(tricyclo[3.3.1.1^{3,7}]dec-1-ylmethyl)-benzamide, 2.Chloro-5-[2-[2-[(2-hydroxyethyl)amino]ethoxy]ethoxy]-N-(tricyclo[3.3.1.1^{3,7}]dec-1-ylmethyl)-benzamide, 2.Chloro-5-[[2-[[2-(1-methyl-1H-imidazol-4-yl)ethyl]amino]ethyl]amino]-N-(tricyclo[3.3.1.1^{3,7}]dec-1-ylmethyl)-benzamide, 2.Chloro-5-piperazin-1-ylmethyl-N-(tricyclo[3.3.1.1]dec-1-ylmethyl)-benzamide, 2.Chloro-5-(4-piperidinyloxy)-N-(tricyclo[3.3.1.1^{3,7}]dec-1-ylmethyl)-benzamide, 2.Chloro-5-(2,5-diazabicyclo[2.2.1]hept-2-ylmethyl)-N-(tricyclo[3.3.1.1]dec-1-ylmethyl)-benzamide, 2.Chloro-5-(piperidin-4-ylsulfinyl)-N-(tricyclo[3.3.1.1^{3,7}]dec-1-ylmethyl)-benzamide, 5.Chloro-2-[3-[(3-hydroxypropyl)amino]propyl]-N-(tricyclo[3.3.1.1^{3,7}]dec-1-ylmethyl)-4-pyridinecarboxamide, 5.Chloro-2-[3-(ethylamino)propyl]-N-(tricyclo[3.3.1.1^{3,7}]dec-1-ylmethyl)-4-pyridinecarboxamide, 5.Chloro-2-[3-[(2-hydroxyethyl)amino]propyl]-N-(tricyclo[3.3.1.1^{3,7}]dec-1-ylmethyl)-4-pyridinecarboxamide, 5.Chloro-2-[3-[[(2S)-2-hydroxypropyl]amino]propyl]-N-(tricyclo[3.3.1.1^{3,7}]dec-1-ylmethyl)-4-pyridinecarboxamide, N-[2-Methyl-5-(9-oxa-3,7-diazabicyclo[3.3.1]non-3-ylcarbonyl)phenyl]-tricyclo[3.3.1.1^{3,7}]decane-1-acetamide, or combinations thereof.

5. Other Agents which Disrupt Electron Transport

In another embodiment, an agent which disrupts electron transport can be used to induce tolerogenicity in dendritic cells. Such agents include, e.g., rotenone, antimycinA, and oligomycin.

6. Combinations of Agents

In another exemplary embodiment, the tolerogenic stimulus comprises or consists of a combination of agents, e.g., a cocktail of agents, for example, more than one of the agents set forth above. Exemplary tolerogenic stimuli include at least one respirostatic or tolerogenic locking agent which can be used to produce induced tolerogenic dendritic cells. In one embodiment, the at least one agent comprises an mTOR inhibitor and a TGFβ agonist. In another embodiment, the at least one agent comprises a statin. In another embodiment, the at least one agent comprises an mTOR inhibitor and a statin. In another embodiment, the at least one agent comprises an mTOR inhibitor, aTGFβ agonist, and a statin. In another embodiment, the at least one agent comprises a purinergic receptor antagonist. In another embodiment, the at least one agent comprises a purinergic receptor antagonist and a statin. In another embodiment, the at least one agent comprises a purinergic receptor antagonist and an mTOR inhibitor. In another embodiment, the at least one agent comprises a purinergic receptor antagonist, an mTOR inhibitor and a TGFβ agonist. In another embodiment, the at least one agent comprises a purinergic receptor antagonist, an mTOR inhibitor, a TGFβ agonist and a statin. In another embodiment, the at least one agent comprises an agent which disrupts mitochondrial electron transport in the DCs. In another embodiment, the at least one agent comprises an agent which disrupts mitochondrial electron transport in the DCs and an mTOR inhibitor. In another embodiment, the at least one agent comprises an agent which disrupts mitochondrial electron transport in the DCs and a statin. In another embodiment, the at least one agent comprises an agent which disrupts mitochondrial electron transport in the DCs, an mTOR inhibitor, and a TGFβ agonist. In another embodiment, the at least one agent comprises an agent which disrupts mitochondrial electron transport in the DCs, an mTOR inhibitor, a TGFβ agonist, and a statin.

In another exemplary embodiment, the tolerogenic stimulus comprises or consists of a combination of agents selected from the group consisting of: i) an mTOR inhibitor (e.g., rapamycin or a variant or derivative thereof); a TGFβ agonist (e.g., TGFβ); ii) a statin; an mTOR inhibitor (e.g., rapamycin or a variant or derivative thereof), a TGFβ agonist (e.g., TGFβ), and a statin; iv) a purinergic receptor antagonist (e.g., oATP); and v) an agent which disrupts mitochondrial electron transport in the DCs (e.g., rotenone).

7. Concentrations of Tolerogenic Stimuli

Exemplary concentrations of tolerogenic stimuli for producing induced tolerogenic cells can be readily determined by a person of skill in the art by titration of the stimulus on a starting population of cells in culture and testing the phenotype of the induced cells ex vivo. In one embodiment, a concentration of agent is chosen which has the desired effect on oxygen consumption rate (i.e., no change in the rate or a reduction in the rate) in dendritic cells. In another embodiment, a concentration of agent is chosen which has the desired effect on the induction of Treg cells. In exemplary embodiments, tolerogenic stimuli are used at a concentrations of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In other embodiments, tolerogenic stimuli are used at concentrations of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 pg/mL, 10 pg/mL, 100 pg/mL, 200 pg/mL, 300 pg/mL, 400 pg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein.

In some embodiments, an mTOR inhibitor (e.g., rapamycin or a derivative or variant thereof) is used as a tolerogenic stimulus at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In exemplary embodiments, an mTOR inhibitor e.g., rapamycin is used at a concentration of 1 μM or 10 nM. In other embodiments, an mTOR inhibitor (e.g., rapamycin or a derivative or variant thereof) is used at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 5 ug/ml, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein.

In some embodiments, one or more statins are used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In another embodiment, a statin is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In an exemplary embodiment, a statin is used at a concentration of about 10, 30, 50, 75, 100, or 300 uM.

In some embodiments, a TGFβ agonist is used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 20 ng/ml, 30 ng/ml, 50 ng/ml, 75 ng/ml, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL and ranges therein. In another embodiment, a TGFβ agonist is used at a concentration of 1 pM to mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM. In exemplary embodiments, TGFβ is used as a tolerogenic stimulus at a concentration of 20 ng/mL.

In some embodiments, a purinergic receptor antagonist (e.g., oATP) is used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In another embodiment, a purinergic receptor antagonist is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein In exemplary embodiments, oATP is used as a tolerogeinc stimulus at a concentration of 100 uM-1 mM.

In some embodiments, an agent which disrupts mitochondrial electron transport is used as a tolerogenic stimulus at a concentration of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In another embodiment, an agent which disrupts mitochondrial electron transport is used at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein.

In one embodiment, when combinations of agents are used, the concentration of each may be reduced.

8. Timing of Exposure

In general, exposure of a starting population of cells comprising dendritic cells and/or dendritic cell precursors to at least one tolerogenic stimulus is of a time sufficient to create induced tolerogenic dendritic cells, e.g., as demonstrated by a tolerogenic phenotype. In one embodiment, cells are contacted with at least one tolerogenic stimulus for at least one hour. In another embodiment, cells are contacted with at least one tolerogenic stimulus for at least two hours. In another embodiment, cells are contacted with at least one tolerogenic stimulus for at least three hours. In another embodiment, cells are contacted with at least one tolerogenic stimulus for at least four hours. In another embodiment, cells are contacted with at least one tolerogenic stimulus for at least five hours. In another embodiment, cells are contacted with at least one tolerogenic stimulus for at least six hours. In another embodiment, cells are contacted with at least one tolerogenic stimulus for at least seven hours. In another embodiment, cells are contacted with at least one tolerogenic stimulus for at least eight hours. In another embodiment, cells are contacted with at least one tolerogenic stimulus for at least eight hours. In another embodiment, cells are contacted with at least one tolerogenic stimulus for at least nine hours. In another embodiment, cells are contacted with at least one tolerogenic stimulus for at least ten hours. In another embodiment, cells are contacted with at least one tolerogenic stimulus for at least eleven hours. In another embodiment, cells are contacted with at least one tolerogenic stimulus for at least twelve hours. In another embodiment, cells are contacted with at least one tolerogenic stimulus for at least thirteen hours. In another embodiment, cells are contacted with at least one tolerogenic stimulus for at least fourteen hours. In another embodiment, cells are contacted with at least one tolerogenic stimulus for at least fifteen hours. In another embodiment, cells are contacted with at least one tolerogenic stimulus for at least sixteen hours.

In another embodiment, cells are contacted with at least one tolerogenic stimulus for from one to seventy two hours, e.g., from two to forty eight hours, from three to twenty four hours, from four to sixteen hours, from five to twelve hours, from four to ten hours, from five to eight hours.

In another embodiment, cells are contacted with at least one tolerogenic stimulus for at least one hour and less than ten hours. In another embodiment, cells are contacted with at least one tolerogenic stimulus for at least two hours and less than ten hours. In another embodiment, cells are contacted with at least one tolerogenic stimulus for at least three hours and less than ten hours. In another embodiment, cells are contacted with at least one tolerogenic stimulus for at least four hours and less than ten hours. In another embodiment, cells are contacted with at least one tolerogenic stimulus for at least five hours and less than ten hours. In another embodiment, cells are contacted with at least one tolerogenic stimulus for at least six hours and less than ten hours. In another embodiment, cells are contacted with at least one tolerogenic stimulus for at least seven hours and less than ten hours. This embodiment, which employs shorter incubation times than previously taught or suggested in the art was used in some, but not all of the appended Examples. In one embodiment, such shorter incubation times are employed for treatment of starting populations of cells comprising or enriched for fully differentiated dendritic cells (i.e., populations of cells which have been treated to differentiate dendritic cell precursors). In one embodiment, such shorter incubation times are employed for treatment of starting populations of cells comprising dendritic cell precursors (i.e., populations of cells which have not been treated to differentiate dendritic cell precursors). In one embodiment, shorter incubation time improves yields of viable cells and can be used for treatment of cells with mTor inhibitors (e.g., rapamycin and variants or derivatives thereof) alone. In addition, these short incubation times can be used to produce tolerogenic dendritic cells using e.g., respirostatic or tolerogenic locking agents.

### V. Induced Immunogenic Dendritic Cells

In one embodiment, the starting population of cells comprising dendritic cells and/or dendritic cell precursors is contacted with one or more agents that induce the DCs in the population to become more immunogenic. Upon treatment with an immunogenic stimulus, DC become capable of increasing the activation of Teff cells. The ability of DC to upregulate the function of effector T cells also provides methods whereby a population of immunogenic DC can be administered to a subject e.g., a subject being vaccinated, having an infection, or having cancer, as discussed below.

In one embodiment, the functional phenotype of the induced immunogenic dendritic cells can be tested to confirm that they are capable of enhancing T effector cell response prior to further manipulation. In another embodiment, the ability of TLR agonists to increase mitochondrial activation in the DCs can be tested prior to further manipulation of induced immunogenic dendritic cells. In contrast to induced tolerogenic dendritic cells, induced immunogenic dendritic cells exhibit a transient increase in mitochondrial activation upon stimulation with one or more TLR agonists.

As set forth above, in one embodiment, oxygen consumption can be used as a readout of mitochondrial activation. The oxygen consumption rate (OCR) can be measured using methods known in the art, e.g., using an analyzer such as the Seahorse XF24 flux analyzer or Clark electrode.

In another embodiment, alternative readouts of mitochondrial activation can be measured. For example, glucose uptake (e.g., using derivatized glucose) or the presence of reactive oxygen species (e.g., using DCF-DA). In another embodiment, lactic acid production (which is elevated with increased glycolysis and/or decreased mitochondrial activity) can be measured. In one embodiment, the extracellular acidification rate (ECAR) can be measured and is reflective of lactic acid production by glycolysis or pyruvate overload. The Seahorse SF24 flux analyzer can be used for this purpose. In yet another embodiment, cellular ATP/ADP ratios may be measured (e.g., using commercially available kits or as in Nagel et al. 2010. Methods Mol. Biol. 645:123-31). Increased levels of ATP and decreased levels of ADP have been recognized in proliferating cells and are a measure of activation.

In another embodiment, the level of expression of a gene which is a marker of mitochondrial activation can be measured. For example, in one embodiment, mRNA levels of the expression of one or more of PGC-1a, PGC-1b, PRC, or other molecules involved in mitochondrial function, such as estrogen-related receptor α, NRF-1, NRF-2, Sp1, YY1, CREB and MEF-2/E-box factors can be measured. As set forth above, in one embodiment, the regulatory region of such a gene or a portion thereof may be operably linked to a reporter gene to facilitate measurement.

### VI. Ex Vivo Production of Induced Immunogenic Dendritic Cells

Induced immunogenic DCs of the invention are produced ex vivo by contacting starting populations of cells comprising dendritic cells and/or dendritic cell precursors with at least one immunogenic stimulus, e.g., as described in more detail below.

Immunogenic Stimuli

The term “immunogenic stimuli” as used herein includes substances which, alone or in combination, improve or enhance the ability of dendritic cells to enhance effector T cell responses. In one embodiment, induced immunogenic dendritic cells increase the proportion and/or activity of antigen specific Teff cells in a cell population. Exemplary immunogenic stimuli include those agents which increase mitochondrial activation (e.g., as measured by oxygen consumption) or which uncouple electron transport in mitochondria. Such exemplary agents include, e.g., at least one Toll-like receptor agonist either alone or in combination with another TLR agonist or maturation stimulus. After treatment with one or more immunogenic stimuli the cells may be removed from the agents, e.g., by centrifugation and/or by washing prior to further manipulation. Immunogenic stimuli of the invention induce a rapid (and transient) increase in the mitochondrial function of dendritic cells as evidenced by an enhanced oxygen consumption rate. Exemplary such stimuli are set forth below:

1. Toll-Like Receptor Agonists

In one embodiment a TLR agonist is known in the art. In another embodiment, a TLR agonist can be identified by performing a screening assay to identify those compounds which result in at least a threshold increase of some biological activity mediated by the TLR of interest. Conversely, a compound may be identified as not acting as an agonist of TLR if, when used to perform an assay designed to detect biological activity mediated by the TLR, the compound fails to elicit a threshold increase in the biological activity. An increase in biological activity refers to an increase in the same biological activity over that observed in an appropriate control.

In one embodiment, a composition comprising an agonist of TLR4 can be used to induce immunogenic dendritic cells. Exemplary such agonists include: lipopolysaccharide or synthetic variants thereof (e.g., MPL and RC529) and lipid A or synthetic variants thereof (e.g., aminoalkyl glucosaminide 4-phosphates). See, e.g., Cluff et al. 2005 Infection and Immunity, p. 3044-3052:73; Lembo et al. The Journal of Immunology, 2008, 180, 7574-7581; Evans et al. 2003. Expert Rev Vaccines 2:219-29.

In one embodiment, a composition comprising an agonist of TLR3 can be used to induce immunogenic dendritic cells. Exemplary such agonists are nucleic acid based molecules, including single-stranded or double-stranded RNA molecules, mixtures thereof, poly I:C, or derivatives thereof, e.g., poly I:C poly Arginine, or polyadenylic-polyuridylic acid, i.e., poly (A): poly (U), polyAU or poly A: U. The nucleotides therein can be natural or synthetic, and may be derivatives or analogs of natural nucleotides, such as for example in Kandimalla et al. ((2003) Nucl. Acid. Res. 31 (9): 2393-2400).

Preferred agonists are double-stranded RNA. The term “double-stranded RNA” molecule designates any therapeutically or prophylactically effective (synthetic) double-stranded RNA compound. dsRNA TLR3 agonists can have any suitable length. Preferably, a dsRNA molecule TLR3 agonist has a length of at least about 10 base pairs (bp), 20 bp, 30 bp, 50 bp, 80 bp, 100 bp, 200 bp, 400 bp, 600 bp, 800 bp or 1000 bp. In one aspect the dsRNA molecule is a short dsRNA having a chain length of less than 30 bp, 50 bp, 80 bp, 100 bp or 200 bp. In another embodiment, the dsRNA molecule is a longer dsRNA, but having a chain length of less than 400 bp, 600 bp, 800 bp or 1000 bp. In another embodiment, the dsRNA molecule is a long dsRNA having a chain length of greater than 1000 bp. In one aspect, a dsRNA composition comprises a heterogenous mixture of dsRNA molecules, wherein a plurality of molecules have differing lengths. Preferably the dsRNA molecules have on average a length of at least about 10 bp, 20 bp, bp, 50 bp, 80 bp, 100 bp, 200 bp, 400 bp, 600 bp, 800 bp or 1000 bp. In another embodiment, a dsRNA composition comprises a plurality dsRNA molecules where at least 20%, 50%, 80%, 90% or 98% of dsRNA molecules have a length of at least about bp, 20 bp, 30 bp, 50 bp, 80 bp, 100 bp, 200 bp, 400 bp, 600 bp, 800 bp or 1000 bp. In a preferred embodiment dsRNA composition has a substantially homogenous mixture of dsRNA molecules, where substantially all the molecules do not differ in chain length by more than 30 bp, 50 bp, 80 bp, 100 bp or 200 bp.

Preferred examples of such dsRNAs are homopolyRNAs, i.e., dsRNAs in which each strand comprises essentially a repeat of the same base; or comprise a homopolyRNA region. The base may be any naturally occurring base (e.g., polyA, polyU, polyC, polyG) or non-naturally occurring (e.g., chemically synthesized or modified) base (e.g., polyI). Polynucleotides typified by polyinosinic—polycytidylic acid, i.e., poly (I): poly(C) or poly I: C and polyadenylic-polyuridylic acid, i.e., poly (A): poly (U) or poly A: U, are well-known compounds in the art and have been known to induce interferon production by immune cells. Thus in one embodiment, the TLR3 agonist for use according to the invention is a double stranded nucleic acid selected from the group consisting of: polyinosinic acid and polycytidylic acid, polyadenylic acid and polyuridylic acid, polyinosinic acid analogue and polycytidylic acid, polyinosinic acid and polycytidylic acid analogue, polyinosinic acid analogue and polycytidylic acid analogue, polyadenylic acid analogue and polyuridylic acid, polyadenylic acid and polyuridylic acid analogue, and polyadenylic acid analogue and polyuridylic acid analogue.

Other examples of dsRNA include nucleic acids described in U.S. Pat. Nos. 5,298,614 and 6,780,429. The disclosures of each of these references is incorporated herein by reference. Other nucleic acid agonists that can be suitable for use as TLR3 agonists are provided in: Field et al.: Proc. Nat. Acad. Sci. U.S. 58, 1004, (1967); Field et al.: Proc. Nat. Acad. Sci. U.S. 58, 2102, (1967); Field et al.: Proc. Nat. Acad. Sci. U.S. 61, 340, (1968); Tytell et al.: Proc. Nat. Acad. Sci. U.S. 58, 1719, (1967); Field et al.: J. Gen. Physiol. 56, 905 (1970); De Clercq et al.: Methods in Enzymology, 78, 291 (1981). A number of synthetic nucleic acid derivatives have been described, including homopolymer-homopolymer complexes (Double Strand Nucleic Acid Polymer such as those in which poly I:C or poly A:U are a parent structure, where these homopolymer-homopolymer complexes contain: (1) base modifications, exemplified by Polyinosinic acid-poly(5-bromocytidylic acid), Polyinosinic acid-poly(2-thiocytidylic acid), Poly(7-deazainosinic acid)-polycytidylic acid, Poly(7-deazainosinic acid)-poly(5-bromocytidylic acid), and Polyinosinic acid-poly(5-thiouridylic acid); (2) Sugar Modifications, exemplified by Poly(2′-azidoinosinic acid)-polycytidylic acid; and (3) Phosphoric Acid Modifications, exemplified by Polyinosinic acid-poly(cytidyl-5′-thiophosphoric acid). Other synthetic nucleic acid derivatives that have been described include interchanged copolymers, exemplified by Poly(adenylic acid-uridylic acid); and homopolymer-copolymer complexes, exemplified by Polyinosinic acid-poly(cytidylic acid-uridylic acid) and Polyinosinic acid-poly(citydylic acid-4-thiouridylic acid). Other synthetic nucleic acid derivatives that have been described include complexes of synthetic nucleic acid with polycation, exemplified by Polyinosinic acid-polycytidylic acid-poly-L-lysinecarboxy-methylcellulose complex (called “Poly ICLC”). Yet another example of synthetic nucleic acid derivative is Polyinosinic acid-poly(1-vinylcytosine).

Yet another example of a TLR3 agonist is Ampligen® (Hemispherx, Inc., of Rockville, Md., U.S.A.), a dsRNA formed by complexes of polyriboinosinic and polyribocytidylic/uridylic acid, such as rIₙ:r (Cₓ, U or G)ₙ where x has a value from 4 to 29, e.g., rIₙ:r (C₁₂ U)ₙ. Many mismatched dsRNA polymers which behave similarly to Ampligen have been studied; mismatched dsRNA based on poly I:C has included complexes of a polyinosinate and a polycytidylate containing a proportion of uracil bases or guanidine bases, e.g., from 1 in 5 to 1 in 30 such bases. The key therapeutic advantage of mismatched dsRNAs over other forms of natural and/or synthetic dsRNAs a reported reduction in toxicity over compounds such as those described in Lampson et al in U.S. Pat. No. 3,666,646.

Specific examples of double-stranded RNA further include Polyadenur (Ipsen) and Ampligen (Hemispherx). Polyadenur is a polyA/U RNA molecule, i.e., contains a polyA strand and a polyU strand. Polyadenur has been developed for the potential treatment of hepatitis B virus (HBV) infection. Ampligen is of a polyI/polyC compound (or a variant thereof comprising a polyI/polyC12U RNA molecule). Ampligen is disclosed for instance in EP 281 380 or EP 113 162. Ampligen has been proposed for the treatment of cancer, viral infections and immune disorders. It was developed primarily for the potential treatment of myalgic encephalomyelitis (ME, or chronic fatigue syndrome/chronic fatigue immune dysfunction syndrome, CFS/CFIDS).

A TLR3 agonist can also be an organic or inorganic substance, such as a lipid, peptide, polypeptide, small molecule, etc., in isolated or in mixture with other substances. The TLR3 agonist candidate may be selected from a combinatorial library of products, for instance. In a preferred embodiment, the TLR3 agonist is an antibody directed against TLR3 receptor and which is capable of activating a TLR3 receptor to induce a full or partial receptor-mediated response. The TLR3 agonist can also be an antibody fragment or derivative of an antibody directed against TLR3 receptor and which is capable of activating a TLR3 receptor to induce a full or partial receptor-mediated response.

In one embodiment, a composition comprising an agonist of TLR9 can be used to induce immunogenic dendritic cells. Exemplary such agonists include CpG oligodeoxynucleotides (ODN) (See, e.g., U.S. Pat. No. 6,194,388). A “CpG motif” as used herein is defined as an unmethylated cytosine-guanine (CpG) dinucleotide.

Many immunostimulatory nucleotide sequences have been described in the art and may readily be identified using standard assays which indicate various aspects of the immune response, such as cytokine secretion, antibody production, NK cell activation and T cell proliferation. See, e.g. U.S. Pat. Nos. 6,194,388 and 6,207,646; WO 98/52962; WO 98/55495; WO 97/28259; WO 99/11275; Krieg et al., 1995, Nature 374:546-549; Yamamoto et al., 1992 J. Immunol. 148:4072-4076; Ballas et al., 1996, J. Immunol. 157 (5) 1840-1845; Klinman et al., 1997, PNAS 93 (7):2879-83; Shimada et al., 1986, Jpn. J. Cancer Res. 77:808-816; Cowdery et al., 1996, J. Immunol. 156:4570-75; Hartmann et al., 2000, J. Immunol. 164 (3):1617-24.

The immunostimulatory nucleotide sequences can by of varying length, lengths greater than 6 bases or base pairs are preferred. An immunostimulatory nucleotide sequence can contain modifications, such as modification of the 3′ OH or 5′ OH group, modifications of a nucleotide base, modifications of the sugar component, and modifications of the phosphate ring. The immunostimulatory nucleotide sequence may be single or double stranded DNA, as well as single or double-stranded RNA or other modified polynucleotides. An immunostimulatory nucleotide sequence may or may not include one or more palindromic regions.

The immunostimulatory nucleotide sequence can be isolated using conventional polynucleotide isolation procedures, or can be synthesized using techniques and nucleic acid synthesis equipment which are well known in the art including, but not limited to, enzymatic methods, chemical methods and the degradation of larger oligonucleotide sequences. (See, for example, Ausubel et al., 1987 and Sambrook et al., 1989).

Examples of immunostimulatory nucleotide sequences that are useful in the methods of the invention include but are not limited to those disclosed in U.S. Pat. No. 6,218,371; U.S. Pat. No. 6,194,388; U.S. Pat. No. 6,207,646; U.S. Pat. No. 6,239,116 and PCT Publication No. WO 00/06588 (University of Iowa); PCT Publication No. WO 01/62909; PCT Publication No. WO 01/62910; PCT Publication No. WO 01/12223; PCT Publication No. WO 98/55495; and PCT Publication No. WO 99/62923 (Dynavax Technologies Corporation), each of which is incorporated herein by reference.

U.S. Pat. No. 6,194,388 (University of Iowa) discloses immunostimulatory nucleic acids which comprise an oligonucleotide sequence including at least the following formula: 5′X₁X₂CGX₃X₄3′ wherein C and G are unmethylated, wherein X₁X₂ are dinucleotides selected from the group consisting of GpT, GpG, GpA, ApA, ApT, ApG, CpT, CpA, CpG, TpA, TpT, and TpG, and X₃X₄ are dinucleotides selected from the group consisting of: TpT, CpT, ApT, TpG, ApG, CpG, TpC, ApC, CpC, TpA, ApA and CpA and wherein at least one nucleotide has a phosphate backbone modification. For facilitating uptake into cells, preferred CpG containing immunostimulatory oligonucleotides are described as being in the range of 8 to 40 base pairs in size. Immunostimulatory oligonucleotides that fall within this formula would be useful in the presently claimed methods.

WO 99/62923 discloses additional examples of immunostimulatory nucleotide sequences that may be used in conjunction with the present invention. In particular, modified immunostimulatory nucleotide sequences comprising hexameric sequences or hexanucleotides comprising a central CG sequence, where the C residue is modified by the addition to C-5 and/or C-6 with an electron-withdrawing moiety are disclosed. Immunostimulatory oligonucleotides can be stabilized by structure modification which renders them relatively resistant to in vivo degradation. Examples of stabilizing modifications include phosphorothioate modification (i.e., at least one of the phosphate oxygens is replaced by sulfur), nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated. Oligonucleotides which contain a diol, such as tetraethyleneglycol or hexaethyleneglycol, at either or both termini have also been shown to be substantially resistant to nuclease degradation (See U.S. Pat. No. 6,194,388 (University of Iowa)).

The immunostimulatory nucleotide sequences may also be encapsulated in or bound to a delivery complex which results in higher affinity binding to target cell surfaces and/or increased cellular uptake by target cells. Examples of immunostimulatory nucleotide sequence delivery complexes include association with a sterol (e.g. cholesterol), a lipid (e.g. a cationic lipid, virosome or liposome), or a target cell specific binding agent (e/g/a ligand recognized by target cell specific receptor). Preferred complexes must be sufficiently stable in vivo to prevent significant uncoupling prior to internalization by the target cell. However, the complex should be cleavable under appropriate conditions within the cell so that the oligonucleotide is released in a functional form (U.S. Pat. No. 6,194,388; WO 99/62923).

In a particularly preferred embodiment, the TLR agonist is an agonist of TLR9, such as described in Hemmi et al., 2000, Nature 408: 740-745 and Bauer et al., 2001, Proc. Natl. Acad. Sci. USA 98: 9237-9242. The known ligands for TLR-9, to date, are unmethylated oligonucleotide sequences containing CpG motifs such as CpG 1668 in the mouse (TCCATGACGTTCCTGATGCT) (SEQ ID NO: 5) and CpG 2006 in man (TCGTCGTTTTGTCGTTTTGTCGTT) (SEQ ID NO: 1) (Bauer et al., 2001, Proc. Natl. Acad. Sci. USA 98: 9237-9242). Additional agonists of TLR9 are set forth below:

(SEQ ID NO: 1)
CpG 2006: TCGTCGTTTGTCGTTTTGTCGTT
(SEQ ID NO: 2)
CPG 2216: GGGGGACGATCGTCGGGGGG
(SEQ ID NO: 3)
AAC-30: ACCGATAACGTTGCCGGTGACGGCACCACG
(SEQ ID NO: 4)
GAC-30: ACCGATGACGTCGCCGGTGACGGCACCACG

2. Agents that Uncouple Mitochondria and Induce Maximal Mitochondrial Respiration.

Oxidation of energy substrates occurs in mitochondria and results in the generation of a proton gradient across the inner mitochondrial membrane that is used by the F₀F₁-ATPase to resynthesize ATP from ADP. Thus oxygen consumption is coupled to ATP synthesis (mitochondrial coupling). If protons bypass the ATP synthase when cycling across the mitochondrial inner membrane, heat is produced instead of ATP (mitochondrial uncoupling). Exemplary uncoupling agents include 2,4-Dinitrophenol (DNP), C₆H₄N₂O₅ and carbonyl cyanide P-(trifluormethoxy)phenylhydrazone (FCCP).

3. Combinations of Agents

In another exemplary embodiment, the immunogenic stimulus comprises or consists of a combination of agents, e.g., a cocktail of agents. In one embodiment, such a combination of agents comprises at least one toll-like receptor agonist. In one embodiment, such a combination of agents comprises a combination or more than one toll-like receptor agonist. In one embodiment, a composition of induction of immunogenic dendritic cells comprises at least two agents that each recognize a different TLR. In one embodiment, a composition of induction of immunogenic dendritic cells comprises at least two agents that each recognize the same TLR.

4. Concentrations of Immunogenic Stimuli

Exemplary concentrations of immunogenic stimuli for producing induced immunogenic dendritic cells can be readily determined by a person of skill in the art by titration of the amount of stimulus necessary to produce induced immunogenic dendritic cells, e.g., as determined by their ability to enhance effector T cell responses. In one embodiment, a concentration of agent is chosen which has the desired effect on oxygen consumption rate (i.e., increasing the rate) in dendritic cells. In another embodiment, a concentration of agent is chosen which promotes the immunogenic phenotype of the induced immunogenic dendritic cells. In exemplary embodiments, immunogenic stimuli are used at a concentrations of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein. In other embodiments, immunogenic stimuli are used at concentrations of 1 pg/mL and 10 mg/mL, for example, 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 100 μg/mL, 200 μg/mL, 300 μg/mL, 400 μg/mL, 500 μg/mL, 600 g/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein.

### VII. Optional Exposure to Antigen

Induced tolerogenic or induced immunogenic dendritic cells of the invention may express an antigen of interest intrinsicly (e.g., the antigen may be an intrinsic antigen such as a germline gene product such as a self protein, polypeptide or peptide), in which case they will not need to be further modified. For example, in one embodiment, where tolerance to an alloantigen is desired, induced tolerogenic DCs which intrinsicly express the alloantigen to which tolerance is desired, will not need to be manipulated to express an antigen of interest.

In one embodiment, in order to modulate an antigen-specific T-cell mediated immune response to an antigen of interest (e.g., an exogeous antigen), dendritic cells which do not already express the antigen of interest such that it can be recognized by T cells are made to express the antigen of interest or are contacted with the antigen of interest, e.g., by being bathed or cultured with the antigen, such that the dendritic cells will display the antigen on their surface for presentation to T cells (e.g., after processing or by directly binding to MHC).

In one embodiment, induced dendritic cells can be directly contacted with e.g., bathed in or pulsed with) antigen. In another embodiment, the cells may express the antigen or may be engineered to express an antigen by transfecting the cells with an expression vector directing the expression of the antigen of interest such that the antigen is expressed and then displayed in the context of MHC molecules on the surface of the DCs.

Accordingly, in one embodiment, prior to, during, and/or following treatment with a tolerogenic or immunogenic stimulus, the cells are exposed to antigen. In one embodiment, before the cells have been induced with either tolerogenic or immunogenic stimuli, the cells are exposed to antigen. In another embodiment, after the cells have been induced with either tolerogenic or immunogenic stimuli, the cells are exposed to antigen. The antigen may be a crude preparation comprising many proteins, polypeptides, and/or peptides (e.g., a lysate or extract) or may comprise one or more purified proteins, polypeptides, or peptides. Such proteins, polypeptides, or peptides can be naturally occurring, chemically synthesized, or expressed recombinantly.

For example, in one embodiment, cells are contacted with an antigen which is heterogeneous, e.g., which comprises more than one protein, polypeptide, or peptide. In one embodiment, such a protein antigen is a cell lysate, extract or other complex mixture of proteins. In another embodiment, an antigen with which cells are contacted comprises or consists of a protein which comprises a number of different immunogenic peptides. In one embodiment, the cells are contacted with the intact antigen and the antigen is processed by the cells. In another embodiment, the cells are contacted with purified components of the antigen, e.g., a mixture of immunogenic peptides, which may be further processed or may bind directly to MHC molecules on the cells.

In one embodiment, the antigen is targeted to surface receptors on DCs, e.g., by making antigen-antibody complexes (Fanger 1996), Ag-Ig fusion proteins (You et al. 2001) or heat shock protein-peptide constructs (Suzue K 1997, Arnold-Schild 1999, Todryk 1999). In another embodiment, non-specific targeting methods such as cationic liposome association with Ag (Ignatius 2000), apoptotic bodies from tumor cells (Rubartelli 1997, Albert 1998a, Albert 1998b), or cationic fusogenic peptides (Laus 2000) can be used.

In some embodiments, the antigen comprises or consists of a polypeptide that can be endocytosed, processed, and presented by dendritic cells. In other embodiments, the antigen comprises or consists of a short peptide that can be presented by dendritic cells without the need for processing. Short peptide antigens can bind to MHC class II molecules on the surface of dendritic cells. In some embodiments, short peptide antigens can displace antigens previously bound to MHC class II molecules on the surface of dendritic cells. Thus, the antigen may be processed by the dendritic cells and presented or maybe loaded onto MHC molecules on the surface of dendritic cells without processing. Those peptide(s) that can be presented by the dendritic cell will appear on the surface in the context of MHC molecules (e.g., class II molecules) for presentation to T cells. This can be demonstrated functionally (e.g., by measuring T cell responses to the cell) or by detecting antigen-MHC complexes using methods known in the art.

In one embodiment, cells are contacted with an antigen comprising more than one protein or more than one polypeptide or more than one peptide and the antigen is not purified to remove irrelevant (unwanted) proteins polypeptides, or peptides and the cells present those antigens which are processed and displayed. In another embodiment, the antigen used to contact dendritic cells comprises or consists of a single short peptide or polypeptide or mixture of peptides or polypeptides that are substantially pure, e.g., isolated from contaminating peptides or polypeptides. Likewise, the antigen can be a single polypeptide or peptide that is substantially pure and isolated from contaminating polypeptides or peptides. Such short peptides and polypeptides can be obtained by suitable methods known in the art. For example, short peptides or polypeptides can be recombinantly expressed, purified from a complex protein antigen, or produced synthetically.

Alternatively, the antigen used to contact cells comprises or consists of a mixture of more than one short peptide or polypeptide, e.g., a mixture of two, three, four, five, six, seven, eight, nine, ten, twenty, thirty, forty, fifty, one hundred or more short peptides or polypeptides. The antigen used to contact cells can also comprise or consist of a more complex mixture of polypeptides. Use of a mixture of short peptides or polypeptides allows for the preparation of an induced dendritic cell population that is capable of modulating an antigen-specific T-cell mediated immune response to a number of distinct peptides or polypeptides. This is desirable when, for example, the immune response to be inhibited is an immune response against a complex antigen, such as food, pollen, dust mites, or particular cell types. In some embodiments, the antigen comprises a cell extract or cell lysate. In other embodiments, the antigen comprises a tissue extract or tissue lysate.

In other embodiments, the antigen is associated with allergic responses. In such embodiments, the antigen with which the dendritic cells are contacted with can comprise one or more allergens (e.g., one or more polypeptides or peptides derived therefrom). In one embodiment, the antigen is a complex antigen, such as: a food protein (e.g., one or more proteins peptides or polypeptides derived from food, such as eggs, milk, wheat, soy, nuts, seeds, fish, shellfish, or gluten), pollen, mold, dust mites, or particular cell types or cells modified by exposure to a drug or chemical.

In other embodiments, the antigen comprises animal matter, such as one or more of animal dander, hair, urine or excrement. In another embodiment, the antigen comprises insect matter.

In other embodiments, the antigen comprises or consists of one or more peptides or polypeptides derived from food. In still other embodiments, the antigen comprises one or more peptides or polypeptides derived pollen. In other embodiments, the antigen comprises one or more peptides or polypeptides derived dust mites. In other embodiments, the antigen comprises one or more peptides or polypeptides derived gluten. In other embodiments, the antigen comprises one or more peptides or polypeptides derived myelin.

In exemplary embodiments, the antigen (or one of the antigens) with which the dendritic cells are contacted in the foregoing methods is an antigen that is targeted by the immune system of a subject with the disease, e.g., targeted by effector T cells, and such targeting contributes to disease progression.

In one embodiment, the antigen is associated with celiac disease (CD). In such embodiments, the antigen with which the dendritic cells are contacted can be derived from wheat, rye, or barley. In exemplary embodiments, the antigen can comprise gluten or gliadin, or portions or mixtures thereof, for example, amino acids spanning from about amino acid 57 to amino acid 73 of A-gliadin.

In other embodiments, the antigen is associated with type I diabetes. In such embodiments, the antigen with which the dendritic cells are contacted can be one or more peptides or polypeptides derived from islet cells of the pancreas, e.g., can be a cell or tissue lysate or extract; a mixture of proteins or polypeptides or peptides; or one or more purified proteins, polypeptides or peptides.

In other embodiments, the antigen is associated with multiple sclerosis. In such embodiments, the antigen with which the dendritic cells are contacted can be one or more peptides or polypeptides derived from neural cell or tissue. For example, the antigen can be derived from axons, dendrites, neuronal cell bodies, oligodendrocytes, glia cells, microglia or Schwann cells. In particular embodiments, the antigen is myelin, or a component thereof, e.g., myelin basic protein.

In other embodiments, the antigen is associated with primary biliary cirrhosis. In such embodiments, the antigen with which the dendritic cells are contacted can be one or more peptides or polypeptides derived from bile duct cells, e.g., as a cell or tissue lysate or extract.

Other antigens that can be used with the methods of the invention can be envisioned by a person of skill in the art. For example, many autoimmune disorders have been associated with particular proteins, although specific peptide antigens important in such immune responses may not yet be known. Since proteins or mixtures of proteins can be used as antigen in the methods of the instant invention, one of skill in the art could readily determine what antigen or antigen mixture to use for loading dendritic cells to modulate immune responses to that particular antigen.

While previous attempts at antigen-specific therapy in humans have been attempted, they have not been successful, at least in part because the molecular identity of the relevant antigen is usually unknown and likely to vary among patients whose diverse HLA repertoires may favor different antigenic peptides. The use of induced dendritic cells as described herein allows the antigen to be present in a crude antigen preparation (e.g., a cell lysate or a protein extract) that may comprise dendritic cell maturation factors. For example, the induced tolerogenic dendritic cells described herein do not become immunogenic upon exposure to such antigen preparations, given their tolerogenically locked phenotype.

In another embodiment, i.e., where enhanced immunogenicity of DCs is desired, the antigen preparation with which the cells are contacted in the foregoing methods comprises an antigen to which an immune response is desired, e.g., an antigen derived from cancer cells, or from a pathogenic agent (e.g., a bacteria, virus, or other pathogenic organism) or toxin.

A wide range of antigen quantities can be used to contact starting populations of cells comprising dendritic cells and/or dendritic cell precursors or induced dendritic cells. For example, in some embodiments, cells are contacted with antigen at concentrations ranging between 1 pg/mL and 10 mg/mL. In exemplary embodiments, dendritic cells are contacted with antigen at 1 pg/mL, 10 pg/mL, 100 pg/mL, 200 pg/mL, 300 pg/mL, 400 pg/mL, 500 pg/mL, 600 pg/mL, 700 pg/mL, 800 pg/mL, 900 pg/mL, 1 ng/mL, 10 ng/mL, 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 10 μg/mL, 30 ug/ml, 100 μg/mL, 200 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, or 10 mg/mL, and ranges therein. In one embodiment, dendritic cells are contacted with 100 pg/mL of antigen. In other embodiments, cells are contacted with antigen at a concentration of 1 pM to 10 mM, for example, 1, 10, 25, 50, 100, 200, 300, 400, 500, 600, 700, 800, 900 or 1000 pM, about 1, 10, 25, 50, 100, 200, 300, 400, 500, 600, 700, 800, 900 or 1000 nM, about 1, 10, 25, 50, 100, 200, 300, 400, 500, 600, 700, 800, 900 or 1000 μM, or about 1, 10, 25, 50, 100, 200, 300, 400, 500, 600, 700, 800, 900 or 1000 mM, and ranges therein.

In one embodiment, starting populations of cells comprising dendritic cells and/or dendritic cell precursors or induced dendritic cells can be cocultured with antigen for a time sufficient to allow display of the antigen on the surface of the cells, e.g., 1-72 hours under appropriate conditions (e.g., 37° C. in 5% CO₂ atmosphere). For example, in one embodiment, cells are cocultured with antigen for about 1-72 hours, e.g., 1, 2, 3, 4, 5, 6, 7, 8, 9, 10, 12, 20, 24, 30, 35, 40, 45, 48, 50, 55, 60, 70, or 72 hours or such other time period which allows for processing and presentation of the antigen by dendritic cells or loading of peptide antigen onto dendritic cells. Such contacting can take place prior to induction of DCs or after induction and prior to further manipulation.

### VIII. Optional Further Manipulation of Cells

In some embodiments, the induced dendritic cells can be contacted with one or more maturation stimuli prior to administration to a subject. Treatment with a maturation stimulus can enhance the antigen presentation capacity of dendritic cells without blocking their tolerogenicity in the case of induced tolerogenic dendritic cells or can further enhance immunogenicity in the case of induced immunogenic dendritic cells. Such maturation stimuli can include, but are not limited to, an adjuvant, a TLR agonist, a CD40 agonist, an inflammasome activator, or an inflammatory cytokine, and combinations thereof. Treatment of dendritic cells with maturation stimuli can be performed before, during, or following induction and/or contacting with antigen.

In one embodiment, mitochondrial respiration of cells can be tested to ensure that treatment with an inducing agent results in an appropriate response. For example, in one embodiment, O2 consumption (the oxygen consumption rate; OCR) by cells can be measured. In this instance, induced immunogenic dendritic cells can be tested to ensure that O2 consumption increases and induced tolerogenic dendritic cells can be tested to ensure that O2 consumption decreases or does not increase. OCR can be measured, e.g., using an analyzer such as the Seahorse XF24 flux analyzer of Clark electrode.

In another embodiment, a different assay can also be used to confirm the effect of an agent on mitochondrial function. For example, in one embodiment, mRNA levels of the expression of one or more of PGC-1a, PGC-1b, PRC, or other molecules involved in mitochondrial function, such as estrogen-related receptor α, NRF-1, NRF-2, Sp1, YY1, CREB and MEF-2/E-box factors can be measured. For example, induced immunogenic dendritic cells exposed to an immunogenic stimulus can be tested to ensure that levels of PGC-1a mRNA increase or induced tolerogenic dendritic cells exposed to a tolerogenic stimulus can be tested to ensure that levels of PGC-1a mRNA do not increase or decrease. Other methods of testing mitochondrial function which are known in the art can also be used for this purpose.

For example, alternative readouts of DC metabolism can be measured. For example, glucose uptake (e.g., using derivatized glucose) can be measured, as can the presence of reactive oxygen species (e.g., using DCF-DA). In another embodiment, lactic acid production (which is elevated with increased glycolysis and/or decreased mitochondrial activity) can be measured. In one embodiment, the extracellular acidification rate (ECAR) can be measured and is reflective of lactic acid production by glycolysis or pyruvate overload. The Seahorse SF24 flux analyzer can be used for this purpose. In yet another embodiment, cellular ATP/ADP ratios may be measured (e.g., using commercially available kits or as in Nagel et al. 2010. Methods Mol. Biol. 645:123-31). Increased levels of ATP and decreased levels of ADP have been recognized in proliferating cells and are a measure of activation.

In another embodiment, the function of induced tolerogenic dendritic cells can be tested ex vivo to ensure that treatment with an inducing agent results in an appropriate cellular response. For example, in one embodiment, the ability of induced tolerogenic dendritic cells to induce Treg cells ex vivo can be measured prior to administration of induced tolerogenic DCs to a subject, e.g., by measuring Foxp3 expression in a population of cells which have been exposed to the induced DCs as described herein.

In another embodiment, whether the induced tolerogenic dendritic cells have at least one of the following properties can be tested ex vivo using methods known in the art and/or described herein i) the ability to convert naïve T cells to Foxp3⁺ T regulatory cells ex vivo; ii) the ability to delete effector T cells ex vivo; iii) the ability to express costimulatory molecules but retain their tolerogenic phenotype upon stimulation with at least one TLR agonist ex vivo; and/or iv) the ability to remain respirostatic upon stimulation with at least one TLR agonist ex vivo.

In another embodiment, the function of induced immunogenic dendritic cells can be tested ex vivo to ensure that treatment with an inducing agent results in an appropriate cellular response. For example, in one embodiment, the ability of induced immunogenic dendritic cells to increase Teff cell numbers (e.g., as measured by staining for cell surface markers) or activation (e.g., as measured by increased proliferation, upregulation of activation markers) or increased effector/memory differentiation (e.g., by measuring cytokine production or cytotoxicity) ex vivo can be measured prior to administration of induced DCs to a subject.

### IX. Methods of Administering Cells

In one embodiment, induced dendritic cells of the invention (e.g., induced tolerogenic dendritic cells or induced immunogenic dendritic cells) are administered to a subject by an appropriate route. In one embodiment, such administration results in a therapeutic benefit to the subject. In one embodiment, induced dendritic cells of the invention can be used to treat a disease or disorder. In another embodiment, induced DCs can be used in the preparation of a medicament for enhancing (induced immunogenic dendritic cells) or suppressing (induced tolerogenic dendritic cells) antigen specific immune responses.

In one embodiment, the subject is a mammal. In one embodiment, the subject is a human. In another embodiment, the subject is a domesticated animal.

In one embodiment, administration can be, for example, parenteral (e.g., by subcutaneous, intrathecal, intraventricular, intramuscular, or intraperitoneal injection, bronchial injection or by intravenous drip); topical (e.g., transdermal, ophthalmic, or intranasal); or pulmonary (e.g., by inhalation or insufflation of powders or aerosols). Administration can be rapid (e.g., by injection) or can occur over a period of time (e.g., by slow infusion or administration of slow release formulations). In another embodiment, induced DC may be administered to recipients by injection into an allograft or into a surgical field into which the allograft is implanted, or any combination thereof.

In one embodiment, the induced dendritic cells of the invention are administered intravenously. In another embodiment, induced dendritic cells of the invention are administered via inhalation. In yet another embodiment, induced dendritic cells of the invention are administered subcutaneously.

In one embodiment, induced dendritic cells of the invention are formulated for administration. Appropriate carriers or vehicles for administration (e.g., for pharmaceutical administration) of cells are compatible with cell viability and are known in the art. Such carriers and may optionally include buffering agents or supplements that promote cell viability. In one embodiment, cells to be administered are formulated with one or more additional agents, e.g., survival enhancing factors or pharmaceutical agents. In one embodiment, cells are formulated with a liquid carrier which is compatible with survival of the cells.

The quantity of induced dendritic cells to be administered to a subject can be determined by one of ordinary skill in the art. In one embodiment, amounts of cells can range from about 10⁵ to about 10¹⁰ cells per dose. In exemplary embodiments, induced dendritic cells are administered in a quantity of about 10⁵, 10⁶, 10⁷, 10⁸, 10⁹, or 10¹⁰ cells per dose. In other exemplary embodiments, intermediate quantities of cells are employed, e.g., 5×10⁵, 5×10⁶, 5×10⁷, 5×10⁸, 5×10⁹, or 5×10¹⁰ cells. In some embodiments, subjects receive a single dose of induced dendritic cells. In other embodiments, subjects receive multiple doses. Multiple doses may be administered at the same time, or they may be spaced at intervals over a number of days. For example, after receiving a first dose, a subject may receive subsequent doses of induced dendritic cells at intervals of 1, 2, 3, 4, 5, 6, 7, 8, 9, 10, 11, 12, 13, 14, 21, 28, 30, 45, 60, or more days. As will be apparent to one of skill in the art, the quantity of cells and the appropriate times for administration may vary from subject to subject depending on factors including the duration and severity of disease. To determine the appropriate dosage and time for administration, skilled artisans may employ conventional clinical and laboratory means for monitoring the outcome of administration, e.g., on progression of a disorder in the subject or on T cell effector function ex vivo. Such means include known biochemical and immunological tests for monitoring and assessing, for example, inflammation, Teffector cell activity, allograft function, etc.

Prophylactic administration of induced dendritic cells can be initiated prior to the onset of disease or therapeutic administration can be initiated after a disorder is established.

In one embodiment, administration of induced tolerogenic DCs is undertaken e.g., prior to receipt of an allograft transplant. In exemplary embodiments, induced tolerogenic dendritic cells are administered at one or more times including, but not limited to, 30, 14, 13, 12, 11, 10, 9, 8, 7, 6, 5, 4, 3, 2, 1, or 0 days prior to allograft transplantation. In addition or alternatively, induced tolerogenic DC can be administered to an allograft recipient following transplantation. In exemplary embodiments, induced tolerogenic dendritic cells are administered at one or more times including, but not limited to, 1, 2, 3, 4, 5, 6, 7, 8, 9, 10, 11, 12, 13, 14, 30, etc. days following allograft transplantation.

In some embodiments, administration of induced tolerogenic dendritic cells can be accompanied by administration of one or more additional agents. For example induced tolerogenic DCs can be administered with one or more immunosuppressive agents. Exemplary immunosuppressive agents that can be used in combination with the induced tolerogenic dendritic cell therapy described herein include, but are not limited to, cytokines such as, for example, interleukin-10, and/or pharmaceutical agents such as, for example, corticosteroids, methotrexate, NSAIDs, fingolimod, natalizumab, alemtuzumab, anti-CD3, cyclosporine A and tacrolimus (FK506). In preferred embodiments, the use of induced tolerogenic dendritic cells will allow for administration of lower doses of general immunosuppressants than the current standard of care, thereby reducing side effects.

In other embodiments, administration of induced immunogenic dendritic cells can be accompanied by administration of one or more immunostimulatory agents. Exemplary immunostimulatory agents that can be used in combination with the induced immunogenic dendritic cell therapy described herein include, but are not limited to, cytokines, adjuvants such as granulocyte colony-stimulating factor (G-CSF), interferons, imiquimod and cellular membrane fractions from bacteria, IL-12, various chemokines, synthetic cytosine phosphate-guanosine (CpG), oligodeoxynucleotides and glucans.

Accordingly, in certain embodiments, the invention provides methods of reducing T effector cell responsiveness to an antigen comprising contacting a population of induced tolerogenic dendritic cells with effector T cells to thereby reduce effector T cell responsiveness to an antigen. In one embodiment, the step of contacting takes place in a subject, the method comprising administering a population of induced tolerogenic dendritic cells to a subject in an amount sufficient to reduce T effector cell responsiveness. In some embodiments, the method optionally includes contacting a starting population cells comprising dendritic cells and/or dendritic cell precursors or induced tolerogenic dendritic cells with an antigen.

In another embodiment, the invention provides methods of improving T effector responsiveness to an antigen in a subject comprising administering a population of induced immunogenic dendritic cells to the subject in an amount sufficient to improve T effector cell responsiveness to the antigen. In one embodiment, the step of contacting takes place in a subject, the method comprising administering a population of induced immunogenic dendritic cells to a subject in an amount sufficient to enhance T effector cell responses. In some embodiments, the method optionally includes contacting a starting population cells comprising dendritic cells and/or dendritic cell precursors or induced immunogenic dendritic cells with an antigen.

In some embodiments, the induced dendritic cells can be derived from the subject to whom the induced dendritic cells will be administered. Accordingly, in some embodiments, the invention provides a method of reducing or increasing antigen-specific T effector cell responsiveness to an antigen in a subject, comprising isolating a starting population of cells comprising dendritic cells or dendritic cell precursors from the subject, contacting the population of cells with a tolerogenic or immunogenic stimulus and, optionally, an antigen, to thereby produce a population of cells comprising induced tolerogenic or immunogenic dendritic cells, and administering the induced dendritic cell population to the subject, e.g., in an amount sufficient to reduce or increase T effector cell responsiveness to the antigen. In other embodiments, the dendritic cells can be derived from one or more than one individual other than or in addition to the subject to whom the induced dendritic cells will be administered.

In one embodiment, induced tolerogenic DC may be generated from an allograft donor (e.g. as described herein) and transfused into the allograft recipient prior to, during or after allotransplantation. In one embodiment, these allogenic induced tolerogenic DC from the organ donor do not need to be contacted with additional antigen, as they intrinsicly express the antigen that will be recognized by the recipient. In yet another embodiment, both donor and recipient DC are induced and are then mixed together so that the recipient DC acquire allo-antigen from the donor DC.

In another embodiment, dendritic cells and/or dendritic cell precursors from a graft recipient are induced with a tolerogenic stimulus and further contacted cells or antigen from the allograft donor. Accordingly, in some embodiments, a cell lysate or cell extract from cells or tissue from the allograft donor, is used to contact dendritic cells and/or dendritic cell precursors prior to induction or induced tolerogenic dendritic cells prior to administration of the induced tolerogenic dendritic cells to an allograft recipient. In other embodiments, a preparation of purified or partially purified polypeptides isolated from cells or tissue from the allograft donor is used as the antigen. In yet another embodiment, DCs can be contacted with whole allogenic cells as the antigen rather than protein extracts. In one embodiment the cell or protein extract is derived from a particular cell or tissue type, e.g., the cell or tissue being transplanted.

Induced tolerogenic dendritic cells can be administered to an allograft recipient prophylactically, prior to receipt of the allograft, in order to prevent an T effector cell response against the allograft. Alternatively or in addition, induced tolerogenic dendritic cells can be administered to an allograft recipient therapeutically, simultaneously with or subsequent to receipt of the allograft, in order to reduce T effector cell responses against the allograft.

Similarly, in another embodiment, bone marrow transplant (BMT) recipients could be transfused with autologous and/or heterologous allo-Ag presenting induced tolerogenic DC (induced as described above) to prevent or treat GvHD.

In one embodiment, induced tolerogenic DCs can be administered to a subject suffering from a disease or disorder mediated, at least in part, by an unwanted immune response, e.g., an unwanted antigen specific effector T cell response. For example, in one embodiment, induced tolerogenic DCs can be administered to a subject having a disease or disorder associated with inflammation. In one embodiment, induced tolerogenic DCs can be administered to a subject having a disease or disorder associated with autoimmunity. In one embodiment, a method of administering the dendritic cells of the invention to a subject results in a desired effect in the subject. For example, the foregoing methods can be used to reduce antigen specific T effector cell responses in a subject with inflammation or having an autoimmune disorder. In one embodiment, the reduction in antigen specific effector T cell responses results in an improvement in the subject's condition.

In one embodiment, the invention provides methods of treating unwanted antigen specific immune responses, e.g., an inflammatory response or an autoimmune disorder, comprising administering a population of induced tolerogenic dendritic cells to a subject in need thereof in an amount sufficient to treat or reduce symptoms. In particular embodiments, the foregoing methods are used to treat or reduce the symptoms of an inflammatory disorder or autoimmune disorder characterized by a detrimental effector T cell immune response to an antigen, e.g., in subjects having a T cell mediated disorder. In these embodiments, the subject has or is at risk of developing an inflammatory disorder or an autoimmune disorder.

In exemplary embodiments, disorders that would benefit from treatment with induced tolerogenic DCs include, but are not limited to, multiple sclerosis, including neuromyelitis optica; type 1 diabetes; celiac disease; primary biliary cirrhosis; rheumatoid arthritis; psoriasis; Behcet's disease; systemic lupus erythrematosus (SLE); allergies, including allergies to antigens derived from plants, animals, microorganisms, drugs, aerosols, chemicals or food, or antigens derived from organic or inorganic matter; celiac disease; thyroiditis; collagenoses; vasculitis; atherosclerosis; myocarditis; allergic asthma; delayed-type hypersensitivity, atopic dermatitis; systemic scleroderma and sclerosis; inflammatory bowel disease (IBD); Crohn's disease; ulcerative colitis; ischemic reperfusion disorders including surgical tissue reperfusion injury, myocardial ischemic conditions such as myocardial infarction, cardiac arrest, reperfusion after cardiac surgery and constriction after percutaneous transluminal coronary angioplasty, stroke, and abdominal aortic aneurysms; cerebral edema secondary to stroke; cranial trauma, hypovolemic shock; asphyxia; adult respiratory distress syndrome; acute-lung injury; Behcet's Disease; dermatomyositis; polymyositis; dermatitis; meningitis; encephalitis; uveitis; osteoarthritis; lupus nephritis; Sjorgen's syndrome; autoimmune thyroid disease, autoimmune liver disease; Addison's Disease; diseases involving leukocyte diapedesis; central nervous system (CNS) inflammatory disorder; amyotrohpic lateral sclerosis (ALS); Guillain-Barré Syndrome; polyneuropathy; multiple organ injury syndrome secondary to septicaemia or trauma; alcoholic hepatitis; bacterial pneumonia; antigen-antibody complex mediated diseases including glomerulonephritis; sepsis; sarcoidosis; immunopathologic responses to tissue/organ transplantation; inflammations of the lung, including pleurisy, alveolitis, vasculitis, pneumonia, chronic bronchitis, bronchiectasis, diffuse panbronchiolitis, hypersensitivity pneumonitis, idiopathic pulmonary fibrosis (IPF), and cystic fibrosis; psoriatic arthritis; neuromyelitis optica, Guillain-Barre syndrome (GBS), and COPD.

In one embodiment, induced immunogenic DCs can be administered to a subject suffering from a disease or disorder mediated, at least in part, by lack of a desired immune response. For example, in one embodiment, induced immunogenic DCs can be administered to a subject having an infection with an infectious agent, e.g., a virus, bacteria, or parasite. In another embodiment, administration to a subject with normal immune responses can be performed, e.g., to increase effector T cell response to an infectious agent or to a vaccine antigen, for example, as an adjuvant.

In other embodiments, the foregoing methods can be used to enhance immune responses in a subject having cancer. For example, the invention provides methods of enhancing Teff responses in a subject having cancer, comprising administering a population of induced immunogenic dendritic cells to a subject in need thereof in an amount sufficient to enhance antigen specific Teff cell responses to a desired antigen.

In one embodiment, a method of administering the dendritic cells of the invention to a subject results in a desired effect in the subject. For example, the foregoing methods can be used to reduce antigen specific T effector cell responses in a subject (in the case of induced tolerogenic dendritic cells) or to enhance antigen specific T effector cell responses in a subject (in the case of induced immunogenic dendritic cells). In one embodiment, the enhancement in antigen specific effector T cell responses results in an improvement in the subject's condition.

### XI. Screening Methods

In one embodiment, the invention pertains to the identification of agents that directly or indirectly enhance mitochondrial function in DC or agents that induce respirostatic tolerance, or tolerogenic locking. As set forth herein, agents that enhance mitochondrial function can be used to produce induced immunogenic DC, while agents that promote respirostatic tolerance or tolerogenic locking can be used to produce induced tolerogenic DC.

In one embodiment, a screening method of the invention employs a cell, e.g., a dendritic cell, such as a naturally occurring mammalian (e.g., mouse or human) dendritic cell or a dendritic cell precursor (e.g., that is naturally occurring or that has been genetically modified). In one embodiment, a cell for use in a screening method has been transfected to express one or more heterologous genes, or is derived from a transgenic animal.

Assays similar to those used to confirm the function of induced tolerogenic DCs or induced immunogenic DCs as set forth herein above can also be used in screening methods to identify new compounds which can be used to induce dendritic cells.

For example, in one embodiment, dendritic cells can be treated with a test agent and the mitochondrial activation of cells can be tested to identify an agent that results in the desired response. For example, agents that increase mitochondrial activation are potentially useful for producing induced immunogenic dendritic cells and agents that reduce or do not increase mitochondrial activation (and in one embodiment, that reduce or inhibit a transient increase in oxygen consumption upon subsequent exposure to a TLR agonist) are potentially useful for producing induced tolerogenic dendritic cells.

For example, in one embodiment, O2 consumption (the oxygen consumption rate; OCR) of cells can be measured as an indicator of mitochondrial activation. In this instance, induced immunogenic dendritic cells can be tested to ensure that O2 consumption increases and induced tolerogenic dendritic cells can be tested to ensure that O2 consumption decreases or does not increase, e.g., upon stimulation with at least one TLR agonist. OCR can be measured, e.g., using an analyzer such as the Seahorse XF24 flux analyzer.

In another embodiment, alternative readouts of DC metabolism can be measured. For example, glucose uptake (e.g., using derivatized glucose) or the presence of reactive oxygen species (e.g., using DCF-DA). In another embodiment, lactic acid production (which is elevated with increased glycolysis and/or decreased mitochondrial activity) can be measured. In one embodiment, the extracellular acidification rate (ECAR) can be measured and is reflective of lactic acid production by glycolysis or pyruvate overload. The Seahorse SF24 flux analyzer can be used for this purpose. In yet another embodiment, cellular ATP/ADP ratios may be measured (e.g., using commercially available kits or as in Nagel et al. 2010. Methods Mol. Biol. 645:123-31). Increased levels of ATP and decreased levels of ADP have been recognized in proliferating cells and are a measure of activation.

In another embodiment, a different assay can also be used to test the effect of a compound on mitochondrial function. For example, in one embodiment, mRNA levels of the expression of one or more of PGC-1a, PGC-1b, PRC, or other molecules involved in mitochondrial function, such as estrogen-related receptor α, NRF-1, NRF-2, Sp1, YY1, CREB and MEF-2/E-box factors can be measured. For example, dendritic cells exposed to an immunogenic stimulus can be tested to ensure that levels of PGC-1a mRNA increase or dendritic cells exposed to a tolerogenic stimulus can be tested to ensure that levels of PGC-1a mRNA decrease. In one embodiment, the regulatory region of such a gene or a portion thereof may be operably linked to a reporter gene.

As used interchangeably herein, the terms “operably linked” and “operatively linked” are intended to mean that the nucleotide sequence is linked to a regulatory sequence in a manner which allows expression of the nucleotide sequence in a host cell (or by a cell extract). Regulatory sequences are art-recognized and can be selected to direct expression of the desired protein in an appropriate host cell. The term regulatory sequence is intended to include promoters, enhancers, polyadenylation signals and other expression control elements. Such regulatory sequences are known to those skilled in the art and are described, e.g., in, Molecular Cloning: A Laboratory Manual, Third Edition CSHL Press (2001). It should be understood that the design of the expression vector may depend on such factors as the choice of the host cell to be transfected and/or the type and/or amount of protein desired to be expressed. Regulatory sequences include those which direct constitutive expression of a nucleotide sequence in many types of host cell, those which direct expression of the nucleotide sequence only in certain host cells (e.g., tissue-specific regulatory sequences) or those which direct expression of the nucleotide sequence only under certain conditions (e.g., inducible regulatory sequences).

A variety of reporter genes are known in the art and are suitable for use in the screening assays of the invention. Examples of suitable reporter genes include those which encode chloramphenicol acetyltransferase, beta-galactosidase, alkaline phosphatase, green fluorescent protein (and its various mutant forms), or luciferase. Standard methods for measuring the activity of these gene products are known in the art.

In one embodiment, the level of expression of the reporter gene in the indicator cell in the presence of the test compound is higher than the level of expression of the reporter gene in the indicator cell in the absence of the test compound and the test compound is identified as a compound that stimulates the expression of PGC-1a, PGC-1b, PRC, and/or one or more other molecules involved in mitochondrial function, such as estrogen-related receptor α, NRF-1, NRF-2, Sp1, YY1, CREB and MEF-2/E-box factors. In another embodiment, the level of expression of the reporter gene in the indicator cell in the presence of the test compound is lower than the level of expression of the reporter gene in the indicator cell in the absence of the test compound and the test compound is identified as a compound that inhibits the expression of one or more of these molecules and, therefore, is an inhibitor of mitochondrial function.

A variety of test compounds can be evaluated using the screening assays described herein. The term “test compound” includes reagents or test agents which are employed in the assays of the invention and assayed for their ability to influence mitochondrial activation. More than one compound, e.g., a plurality of compounds, can be tested at the same time for their ability to modulate mitochondrial respiration or gene expression in a screening assay. The term “screening assay” preferably refers to assays which test the ability of a plurality of compounds to influence the readout of choice rather than to tests which test the ability of one compound to influence a readout. Preferably, the subject assays identify compounds not previously known to have the effect that is being screened for. In one embodiment, high throughput screening can be used to assay for the activity of a compound.

In certain embodiments, the compounds to be tested can be derived from libraries (i.e., are members of a library of compounds). While the use of libraries of peptides is well established in the art, new techniques have been developed which have allowed the production of mixtures of other compounds, such as benzodiazepines (Bunin et al. (1992). J. Am. Chem. Soc. 114:10987; DeWitt et al. (1993). Proc. Natl. Acad. Sci. USA 90:6909), peptoids (Zuckermann. (1994). J. Med. Chem. 37:2678), oligocarbamates (Cho et al. (1993). Science. 261:1303-), and hydantoins (DeWitt et al. supra).

Exemplary methods used to generate molecular diversity are well known in the art and many reviews have been published, e.g., Shreiber, S. 2009 Nature 457, 153-154; Barry, C. E. I. (2003), 2, 137-150.; Braeckmans, K. et al. (2003) Encoded microcarrier beads signal the way to better combinatorial libraries and biological assays. Mod. Drug Dis., 6, 28-30, 32; Charmot, D. (2003) Actualite Chimique, 11-16; Edwards, P. J. (2003), 6, 11-27; Fassina, G., & Miertus, S. (2003) Chimica Oggi, 21, 28-31; Hermkens, P. H. H., & Muller, G. (2003). Ernst Schering Research Foundation Workshop, 42, 201-220.; Hisamoto, H., Kikutani, Y., & Kitamori, T. (2003) Microchip-based organic synthesis. Shokubai, 45, 252-256; Hughes, D. (2003). Nature Reviews Genetics, 4, 432-441; Jensen, K. J., & Nielsen, J. (2003) Bioorganic and combinatorial chemistry. Part 1. Dansk Kemi, 84, 21-24; Kobayashi, N., & Okamoto, Y. (2003) Farumashia, 39, 769-773.; Lam, K. S., Liu, R., Miyamoto, S., Lehman, A. L., & Tuscano, J. M. (2003). Account. Chem. Res., 36, 370-377; Langer, T., & Krovat, E. M. (2003), 6, 370-376; Liu, R., Enstrom, A. M., & Lam, K. S. (2003). Experimental Hematology (New York, N.Y., United States), 31, 11-30.; Mario Geysen, H., Schoenen, F., Wagner, D., & Wagner, R. (2003) Nature Reviews Drug Discovery, 2, 222-230; Nefzi, A., Ostresh, J. M., & Houghten, R. A. (2003). EXS, 93, 109-123.; New, D. C., Miller-Martini, D. M., & Wong, Y. H. (2003). Phytotherapy Research, 17, 439-448. Pinilla, C., Appel, J. R., Borras, E., & Houghten, R. A. (2003) Nature Medicine (New York, N.Y., United States), 9, 118-122; Schwardt, O., Kolb, H., & Ernst, B. (2003) Current Topics in Medicinal Chemistry (Hilversum, Netherlands), 3, 1-9.; Sehgal, A. (2003). Curr. Med. Chem., 10, 749-755. The contents of these reviews are incorporated by reference herein.

The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422-; Horwell et al. (1996) Immunopharmacology 33:68-; and in Gallop et al. (1994); J. Med. Chem. 37:1233-.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310). In still another embodiment, the combinatorial polypeptides are produced from a cDNA library. In one embodiment, cDNA molecules for testing can be expressed in viral libraries, e.g., be retro-, lenti-, or adenoviral libraries. In another embodiment, RNAi libraries developed using methods known in the art can be screened.

Exemplary compounds which can be screened for activity include, but are not limited to, peptides, nucleic acids, carbohydrates, small organic molecules, and natural product extract libraries.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); 5) enzymes (e.g., endoribonucleases, hydrolases, nucleases, proteases, synthatases, isomerases, polymerases, kinases, phosphatases, oxido-reductases and ATPases), or RNAi molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Other examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In another embodiment, the effect of the compound of interest on the cells, is compared to an appropriate control (such as untreated cells or cells treated with a control compound, or carrier, that does not modulate the biological response).

In another embodiment, a test compound is identified that or that directly or indirectly modulates mitochondrial respiration, e.g., by one of the variety of methods described hereinbefore, the selected test compound (or “compound of interest”) can then be further evaluated in a secondary screening assay.

In one embodiment, compounds found to modulate mitochondrial respiration are further tested for their ability to modulate the ability of DC to affect T cells by, e.g., testing their ability to convert naïve T cells into Treg ex vivo, to delete effector cells ex vivo, or, conversely to induce T cell activation ex vivo (as measured, e.g., by increased proliferation and/or upregulation of activation markers) and/or effector/memory differentiation ex vivo (as measured, e.g., by increased cytokine production, cytotoxicity etc.) e.g., using methods known in the art.

Compounds identified in the subject screening assays can be used in methods of modulating induction of dendritic cells. It will be understood that it may be desirable to formulate such compound(s) as pharmaceutical compositions (described supra) prior to contacting them with cells.

### EXAMPLES

The following materials and methods were used in the Examples:

### Mice and Mouse Cells

C57/B6, OTIIxRAG1−/−, CX3CR1-GFP, Fas ligand deficient (FasLKO), Indoleamine-pyrrole 2,3-dioxygenase deficient (IDOKO), IL-6KO, IL-10KO and tuberous sclerosis protein 1 conditional knock out (TSCllox/lox) mice were obtained from Jackson Laboratories or Taconic Farms.

The following mouse strains that have been described in the art were used: TCR transgenic (Bettelli, E., M. Pagany, H. L. Weiner, C. Linington, R. A. Sobel, and V. K. Kuchroo. 2003. Myelin oligodendrocyte glycoprotein-specific T cell receptor transgenic mice develop spontaneous autoimmune optic neuritis. The Journal of experimental medicine 197:1073-1081); Foxp3GFP reporter mice (Bettelli E, Carrier Y, Gao W, Korn T, Strom T B, Oukka M, Weiner H L, Kuchroo V K. Reciprocal developmental pathways for the generation of pathogenic effector TH17 and regulatory T cells. Nature. 2006 May 11; 441 (7090):235-8); L7 TCR transgenic mice (Maloy, K. J., C. Burkhart, G. Freer, T. Rülicke, H. Pircher, D. H. Kono, A. N. Theofilopoulos, B. Ludewig, U. Hoffmann-Rohrer, R. M. Zinkernagel, and H. Hengartner. 1999. Qualitative and quantitative requirements for CD4+ T cell-mediated antiviral protection. J Immunol 162:2867-2874); CD274 deficient (PDL1−/−), CD273 deficient (PDL2−/−) and CD274/273 double deficient animals (PDL1L2−/−) (Francisco, L. M., V. H. Salinas, K. E. Brown, V. K. Vanguri, G. J. Freeman, V. K. Kuchroo, and A. H. Sharpe. 2009. PD-L1 regulates the development, maintenance, and function of induced regulatory T cells. The Journal of experimental medicine 206:3015-3029); CCR2-RFP reporter animals (Saederup et al. Selective chemokine receptor usage by central nervous system myeloid cells in CCR2-red fluorescent protein knock-in mice. PLoS ONE (2010) vol. 5 (10) pp. e13693); and Epstein-Barr-virus-induced gene 3 deficient animals (EBI3KO, Collison et al. The inhibitory cytokine IL-35 contributes to regulatory T-cell function. Nature (2007) vol. 450 (7169) pp. 566-9).

Naïve CD4+ T cells were isolated from single cell suspensions of lymph nodes (LN) and spleens of young mice (6 to 8 weeks) of different genetic backgrounds and enriched using a CD4+ T cell enrichment kit for magnetic negative selection (MACS, Miltenyi). When indicated in the text or figures, T cells from Foxp3GFP reporter animals were purified by MACS negative selection followed by staining with anti-CD4 APC-Cy7 (GK1.5), anti-CD25-PerCPCy5.5 (PC61), anti-CD62L-APC monoclonal antibodies (MEL-14) and anti-CD44 PE-Cy7 (IM7) to isolate CD4+CD25−CD62LhighCD44lowFoxp3GFP− cells using a FACSAria system (BD Biosciences). Alternatively, Tn were obtained from RAG1 deficient animals (RAG1−/−) in which memory T cells and endogenous natural-occurring Treg (nTreg) are absent. MACS-purified CD4+ T cells (whether from wild type, transgenic or RAG1−/− animals) and FACS sorted CD4+CD25−CD62LhighCD44lowFoxp3GFP− will be referred to as Tn.

In some cases, Tn were labeled with the cytoplasmic dye carboxyfluorescein diacetate succinimidyl diester (CFSE, Invitrogen) by incubation for 10 min at 37° C. with CFSE in RPMI medium containing 10% FCS in order to trace their proliferation ex vivo (1 μM) and in vivo (10 μM).

### Preparation of Human Cells

Whole peripheral blood mononuclear cells (PBMC) were isolated from healthy individuals after informed consent in green-capped, heparinized tubes by Ficoll-Hypaque (GE Healthcare) gradient centrifugation. Naive CD4+ T cells were isolated from PBMC via negative isolation kit (MACS, Miltenyi Biotec). Around 94% of the purified cells were CD4+CD25−Foxp3GFP−CD62L+CD44low. In order to obtain monocyte-derived DC (BMDC), monocytes were purified from PBMC by negative selection using magnetic negative selection (MACS, Miltenyi Biotec) and cultured for 6 days in presence of GM-CSF (100 ng/ml) and IL-4 (15 ng/ml). After this culture around 90% of harvested cells were CD11c+ MHC CLII+ MoDC. In order to induce tolerogenic MoDC (itMoDC), MoDC cultures were supplemented with rapamycin (1 nM) and TGFβ (2 ng/ml) for the last two days of the culture. In order to obtain immunogenic MoDC, monocyte cultures were supplemented with LPS (lpsMoDC, 1 μg/ml) the last day of the culture.

### Antibodies and Reagents

The following antibodies against mouse antigens were obtained from BD Biosciences: anti-CD3 (145-2c11), anti-CD103-PE (M290), anti-CD152-PE (UC10-4F10-11), anti-IL10R (1B1.3a), anti-IL10 (JES5-16E3), anti-IAb-PE (AF6-120.1), anti-CD80-PE (16-10A1), anti-CD86-PE (GL1), anti-CD195 (CCR5, C34-3448), anti-CD11c-FITC (HL-3), anti-CD317-APC (PDCA-1, JF05-1C2.4.1), anti-CD197-PE (CCR7, 4B12), anti-CD8α-APCCy7 (53-6.7), anti-CD62LAPC (MEL-14), anti-CD4 APCCy7 (GK1.5) and anti-CD25-PerCPCy5.5 (PC61).

The following antibodies against mouse antigens were obtained from eBiosciences: anti-Foxp3-Alexa 647 (FJK-16s), anti-CD274-PE (PDL1, MIH5), anti-CD273-PE (PD-L2, TY25), anti-CD275-PE (ICOSL, HK5.3), anti-41BBL-PE (TKS-1), anti-CD11b-PECy7 (M1/70) and anti-CD44 PE-Cy7 (IM7). Anti-GITR-PE-Cy7 (YGITR 765) was obtained from Biolegend, and anti-TGFβ (1B11) was obtained from R&D Systems.

The following antibodies against human antigens were obtained from eBioscience: anti-HLA-DR (L243), anti-Foxp3 (206D), anti-CD4 (RPA-T4), anti-CD11c (3.9), anti-CD14 (61D3), anti-CD25 (B696), anti-CD40 (5C3), anti-CD45RO (UCHL1) anti-CD62L (Dreg 56), anti-CD80 (2D10) and anti-CD83 (HB15e).

The following antibodies against human antigens were obtained from BD Biosciences HLA-ABC (G46-2.6), anti-CD1a (HI149), anti-CD3 (OKT3), anti-CD32 (anti-FcRII,), anti-CD54 (HA58), anti-CD86 (2331), anti-CD137L (41BBL, C65-485) anti-CD196 (CCR6, 11A9), anti-CD197 (CCR7, 3D12), anti-CD205 (DEC-205, MG38), anti-CD209 (DC-SIGN, DCN46), anti-CD273 (PD-L2, MIH18) and anti-CD274 (PD-L1, MIH1).

The following antibodies were obtained from Cell Signaling technology for western blots analysis: anti-Akt (5G3), anti-p308Akt (244F9), anti-p473Akt (193H12), anti-p389S6K (108D2), anti-S6K (49D7), pS6 (D57.2.2E), anti-S6 (54D2), anti-4EBP1 (53H11), anti-p4EBP1 (174A9).

Aphidicollin, Rapamycin, All-trans Retinoic Acid, LPS, Atorvastatin, Pravastatin, Periodate-oxidized ATP (oATP), 5-aminoimidazole-4-carboxamide-1-β-4-ribofuranoside (AICAR), suranim, carbonyl cyanide P-(trifluoromethoxy)phenylhydrazone (FCCP), oligomicin, 2′,3′-O-(4-benzoyl)benzoyl ATP (BzATP) and rotenone were obtained from Sigma. TGFβ was obtained from R&D, and recombinant human IL-2 was obtained from Roche. Zymozan, Pam3Cys, Poly I:C, Flagellin, Imiquimod, R848, CL097 and CpG were obtained from Invivogen (San Diego, Calif.).

### Oxygen Consumption Measurements

Mitochondria oxygen consumption rate (OCR) was measured by using a YSI 5300 Clark-type electrode (Yellow Springs Instrument, Yellow Springs, Ohio, USA) or the XF24 Extracellular Flux Analyzer (Seahorse Bioscience, Billerica, Mass.) following the manufacturer's protocol. Primary cultured DC (10⁶ cells/well) were used as described in the experiments.

### Mouse Dendritic Cell Purification and Treatment

Dendritic cells (DC) were purified from the spleen of animals. In some cases these animals were inoculated with a melanoma cell line that produces FLT3-ligand in order to expand DC (FLT3Lm). The spleens of these animals were harvested and digested for 30 min at 37° C. using Liberase CI from Roche (250 μg/ml). Cell suspensions were obtained from spleens dissociated in cold HBSS supplemented with EDTA. The cells were sorted using double magnetic positive selection (MACS) to reach a purity of about 98% CD11c+. DC were incubated for different periods of time under tissue culture conditions (37° C., 5% CO2) or on ice with different combinations of LPS (1 μg/ml), Rapamycin (10 nM), TGFβ (2 ng/ml, Rapa/TGF), All-trans Retinoic Acid (100 nM), purinergic receptor agonists ATP (1 mM) and Bz-ATP (100 μM), purinergic receptors antagonists oATP (100 μM) and suranim (100 μM), the adenosine monophosphate kinase stimulator AICAR (100 μM) and the enzyme Apyrase (200 U/ml) that catalyses ATP, the mitochondrial modifiers FCCP (1 μM) and rotenone (1 μM), Atorvastatin (100 nM) and Pravastatin (100 nM). Cells were washed extensively (up to 6 times) and used to activate Tn. When appropriate, DC were loaded with Ovalbumin protein (OVAp, 100 μg/ml), Ovalbumin peptide (OVA₃₂₃₋₃₃₉, 1 μM), Myelin Oligodendrocyte peptide (MOG₃₅₋₅₅, 10 μg/ml) or VSV-G₄₁₅₋₄₃₃ peptide (1 μM).

In some experiments DC were derived from bone marrow progenitors (BMDC). Bone marrow precursors were harvested from the pooled femurs and tibiae of female mice (8 wk old; five mice per genotype per independent experiment) by flushing with ice-cold complete RPMI 1640 culture medium supplemented with 20 mM HEPES buffer, 2 mM L-glutamine, 2.5 pg/ml gentamicin sulfate, and 8% (v/v) FBS; aggregates were gently disbursed by repeated pipetting in ice-cold culture medium. Cells were centrifuged at 400×g for 10 min at 8° C. and, following two washes in ice-cold divalent cation-free PBS (pH 7.4), the cells were resuspended and the erythrocytes were removed by lysis in ACK buffer (150 mM NH4Cl, 1.0 mM KHCO₃, and 0.1 mM Na₂EDTA, pH 7.4) for 3 min at room temperature. The lysis reaction was quenched by the addition of ice-cold culture medium and centrifugation at 400×g for 10 min at 8° C. Cells were resuspended in PBS containing 10 mM EDTA, 0.1% BSA, and 10 mM HEPES, and centrifuged twice at 200×g for 10 min at 8° C. to deplete platelets. Cell pellets were next resuspended in culture medium and seeded into 6-well tissue culture clusters at a density of 2.5×10⁵ cells per well in a total volume of 4 ml. Cells were cultured at 37° C. in a sterile filtered atmosphere of 5% CO₂/95% air and a fully humidified incubator. Cultures were pulsed at day 0 and every 48 h with a combination of IL-4 (10 ng/ml) and GM-CSF (25 ng/ml) to propagate immature myeloid DC. After 8 days of culture, immature DCs were harvested, washed, and seeded at a density of 8×10⁵ cells/ml in 12-well culture dishes in a total volume of 2.0 ml. A variant of this procedure using FLT3L (100 ng/mL, Peprotech, Rocky Hill, N.J.) for 9 days instead of GM-CSF for 8 days was used as well.

### Ex Vivo Treg Differentiation and Intracellular Staining

Mouse or human Tn were activated in cell culture conditions (3×10⁶ cells/ml, 37° C., 5% CO₂ in RPMI 10% FCS, non-essential amino acids, Hepes buffer, Penicillin and Streptomycin) by mixing them with DC (3×10⁵ cells/ml) in presence of activating anti-CD3 (2c11 for mouse and OKT3 for human cells) when using polyclonal Tn from wild type animals and human cells. Tn from TCR transgenic animals were activated using protein or peptide-loaded DC (as described above).

At different time points during this coculture, cells were washed and prepared to assess their phenotype. For surface staining, cells were washed twice with PBS and PBS-FCS 1% or PBS-BSA 2% (wash media) sequentially and stained in the same media. For intracellular staining, cells were washed, permeabilized, and stained using a kit from BD Biosciences according to the manufacturer's instructions. Nonspecific staining was blocked with Fc receptor blocking Ab (CD16/CD32). Cells were washed twice in staining buffer, and data were acquired using a FACSCanto. Various ratios of itDC to Tn can be used to induce Treg differentiation ex vivo. For example, itDC can be co-cultured with Tn at a ratio of 1:100, 1:10, 1:1, 10:1, or 100:1, and ranges therein. In exemplary embodiments, itDC are co-cultured with Tn at a ratio of 1 DC:10 Tn.

### Mouse Suppression Assays

Tn from the transgenic-reporter strain CD45.2xOTIIxFoxp3GFP were FACS sorted as indicated previously and mixed with OVA₃₂₃₋₃₃₉-loaded CD45.2+ itDC or ctrlDC supplemented with TGFβ and IL-2 for 5 days. GFP−CD11c−7AAD− (effector, Teff cells) and GFP+CD11c−7AAD− cells (Treg) were then FACS purified and mixed with freshly isolated CD45.1+CFSE-labeled Tn in presence of OVA₃₂₃₋₃₃₉-loaded untreated DC. Proliferation was assessed 4 days later by CFSE dilution assay.

### Human Suppression Assays

Human MoDC were isolated and treated as described before and used to activate autologous or allogenic Tn (in presence of anti-CD3). Five days later the cells were harvested and the CD25high (Treg) and CD25low-neg (Teff) contained in the 7AAD−CD11c−CD4+ cells were FACS sorted. Autologous cryopreserved PBMC (in 10% DMSO, 90% FCS) were used to produce a new batch of Tn and ctrlMoDC. Similar to the mouse experiments, these Tn were CFSE labeled and activated by ctrlMoDC (and anti-CD3) in presence or not of activated Treg or Teff. Four days later the proliferation of Tn was assessed by FACS by gating on 7AAD−CD11c−CD4+CFSE+ cells.

### Mixed Leukocyte Reaction

Splenic untreated Balb/c DC (presenters) were mixed with CFSE-labeled C57BL/6 Tn (responders; 10⁵ cells of each population). Differentially treated Balb/cxC57BL/6 (F1) DC (10⁴/well) were added to some wells. After 4 days, proliferation of the responders CD4+TCRβ+H−2D^{b}+7AAD−CD11c− cells was measured by CFSE dilution analyzed by FACS.

### Transwell Experiments

In some experiments, co-cultures were repeated using a transwell system (Tissue Culture Transwell Plates, 0.4-μm pore size; Costar, Milan, Italy) to determine whether direct contact was necessary between itDC and Tn for Foxp3 upregulation. DC were isolated and treated as described before to activate Tn. Different combinations of DC-T cocultures were used in the top (insert) or bottom compartment.

### Model of Multiple Sclerosis in Mice: EAE Protocol

C57/BL6 animals were induced with EAE following standard protocols by injecting 100 μg/mouse of MOG₃₅₋₅₅ emulsified in CFA subcutaneously, and 100 ng/mouse of pertussis toxin (PTx) was injected intravenously at day 0 and 2. EAE scoring included the following guidelines. Score 1: flaccid tail. Score 2: inability to right, weak gait. Score 3: one limb paralysis. Score 4: two limb paralysis. Score 5: moribund. Score 6: death.

### Model of OVA-Induced Experimental Asthma in Mice

Mice (C57BL/6; 6-8 w.o.) were sensitized by i.p. injection of 10 μg of OVA (Sigma-Aldrich) adsorbed overnight at 4° C. to 4 mg of aluminum hydroxide (Sigma-Aldrich) in a total volume of 0.5 ml of sterile PBS as reported (Ohkawara Y, Lei X F, Stampfli M R, Marshall J S, Xing Z, Jordana M. Cytokine and eosinophil responses in the lung, peripheral blood, and bone marrow compartments in a murine model of allergen-induced airways inflammation. Am J Respir Cell Mol. Biol. 1997 May; 16 (5):510-20). Mice were sensitized twice, 5 days apart, and 8 days later challenged on three consecutive days with OVAp (100 μg) via intranasal administration. 48 h prior to the start of OVAp challenges, sensitized mice were treated with itDCs or ctrlDC (10×106 i.v.). Mice were sacrificed 72 h post the 3rd OVA challenge and bronchoalveolar lavage (BAL) was performed as previously described (Alvarez D, Harder G, Fattouh R, Sun J, Goncharova S, Stampfli M R, Coyle A J, Bramson J L, Jordana M. Cutaneous antigen priming via gene gun leads to skin-selective Th2 immune-inflammatory responses. J. Immunol. 2005 Feb. 1; 174 (3):1664-74). Briefly, the lungs were dissected, the trachea was cannulated with a polyethylene tube (BD Biosciences), and the lungs were lavaged twice with PBS (0.5 ml). Total BAL cell counts were determined in a blinded manner using a hemocytometer and smears were prepared by cytocentrifugation (Shandon) at 300 rpm for 2 min. BAL smears were stained with the Protocol Hema 3 stain set (Fisher-Scientific). Differential cell counts of BAL smears were determined in a blinded manner from at least 300-500 leukocytes using standard hemocytological criteria to classify the cells as neutrophils, eosinophils, or mononuclear cells. BAL cells were also stained for flow cytometry for enumeration of eosinophils (CD11b+, SSC-hi, Gr1-lo) and T cells (CD3+, CD4+).

### Model of HDM-Induced Experimental Asthma in Mice

Mice (Balb/c 6-8 w.o.) were sensitized to house dust mite (HDM) extract as reported previously (Fattouh R, Al-Garawi A, Fattouh M, Arias K, Walker T D, Goncharova S, Coyle A J, Humbles A A, Jordana M. Eosinophils are dispensable for allergic remodeling and immunity in a model of house dust mite-induced airway disease Am J Respir Crit Care Med. 2011 Jan. 15; 183(2):179-88). HDM extract (Greer Laboratories, Lenoir, N.C.) was resuspended in sterile saline (2.5 mg of protein/ml), and 10 μl was administered intranasally to isoflurane-anesthetized mice (25 μg total HMD extract protein per intranasal administration). Mice were exposed to HDM extract via intranasal administration for 5 consecutive days followed by 2 days of rest for a total of weeks. DC were harvested and treated with rapamycin and TGFβ as described above and loaded with HDM (30 μg/ml). Intravenous DC therapy (treated or control) commenced 24 h after the first week of HDM exposure and continued every week throughout the experimental protocol for a total of four DC treatment periods (10×10⁶ DC i.v. delivered during each treatment period). Lung function measurements were performed in DC-treated and untreated mice as well as naïve control mice, 24 hours following the 5th week of HDM exposure as described below.

### Measurement of Airway Hyperresponsiveness (AHR) in Mice.

Lung function measurements were performed using the Flexivent system (Scireq, Montreal, Quebec, Canada) to determine airway hyperresponsiveness (Fattouh R, Al-Garawi A, Fattouh M, Arias K, Walker T D, Goncharova S, Coyle A J, Humbles A A, Jordana M. Eosinophils are dispensable for allergic remodeling and immunity in a model of house dust mite-induced airway disease. Am J Respir Crit Care Med. 2011 Jan. 15; 183 (2):179-88). Mice were anesthetized with xylazine (12 mg/kg i.p.) and pentobarbital (70 mg/kg i.p.). Anesthetized mice were tracheostomized, cannulated, and ventilated with 6 ml/kg tidal volumes at 150 breaths per minute. To suppress spontaneous breathing during measurements of lung function, anesthetized mice were injected with pancuronium bromide (2 mg/kg i.p.). Incremental doses of nebulized methacholine (0, 3.13, 12.5, 25, and 50 mg/ml in saline) were used to determine total lung resistance (cmH2O.s/ml) according to the Snapshot-150 perturbation method. For each methacholine dose thirteen data points were collected, and only data with a coefficient of determination greater than 0.93 were included in analyses. The survival of mice during the procedure was simultaneously monitored using electrocardiograms and mice that died during the analysis were omitted from analysis. Resistance is represented as percent change over baseline resistance measurements determined following nebulized saline.

### Example 1

### Differentiation of Foxp3+ Tregs by Induced Tolerogenic DC Ex Vivo

Tolerogenic compounds were screened for their capacity to imprint DC that induce Tn to acquire Treg phenotype and function. The primary criteria to select DC with tolerogenic function consisted of a phenotypic readout of Foxp3 upregulation on T cells to identify suitable candidates. A combination of rapamycin and TGFβ was found to have a significant superior capacity to induce tolerogenic DC that provoke Foxp3 expression on Tn. These T cells had the phetotypic and functional (suppression) characteristics of Treg. Importantly, the induced tolerogenic DC (itDC) were able to upregulate Foxp3 expression in the input population of Tn even if proliferation was prevented using inhibitors of the cell cycle or a strong TCR agonist such as activating anti-CD3 was provided.

a) Screening Strategy to Identify Induced Tolerogenic DC.

DC were harvested from the spleen of normal animals or animals inoculated with melanoma cell lines expressing FLT3L to expand DC in vivo. After various tolerogenic treatments these DC were used to stimulate Tn for various amounts of time ex vivo in presence of a TCR stimulus (antigen or polyclonal activating anti-CD3). Expression of Foxp3 is indicative of the tolerogenic function of DC.

b) Screening of Compounds.

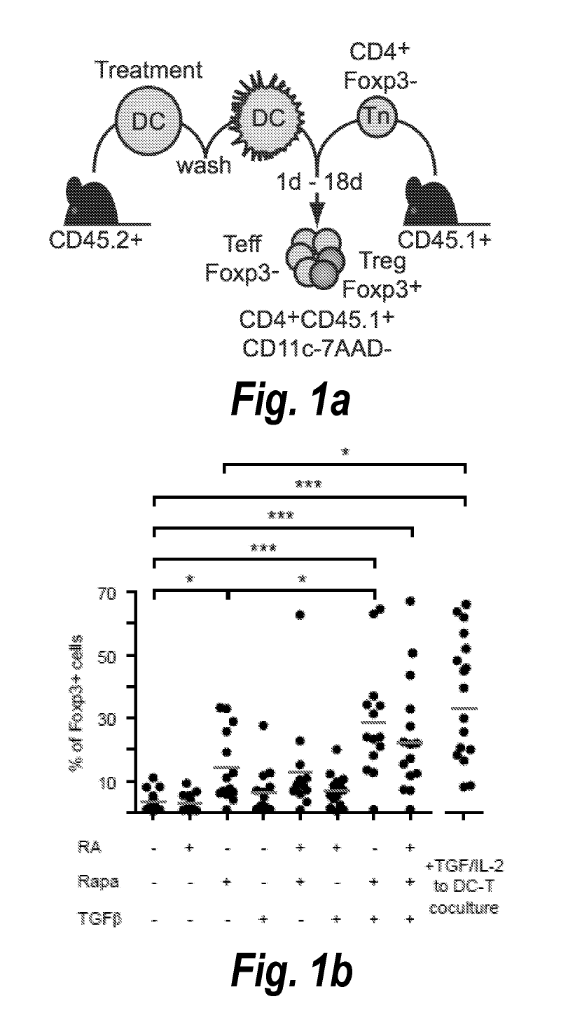

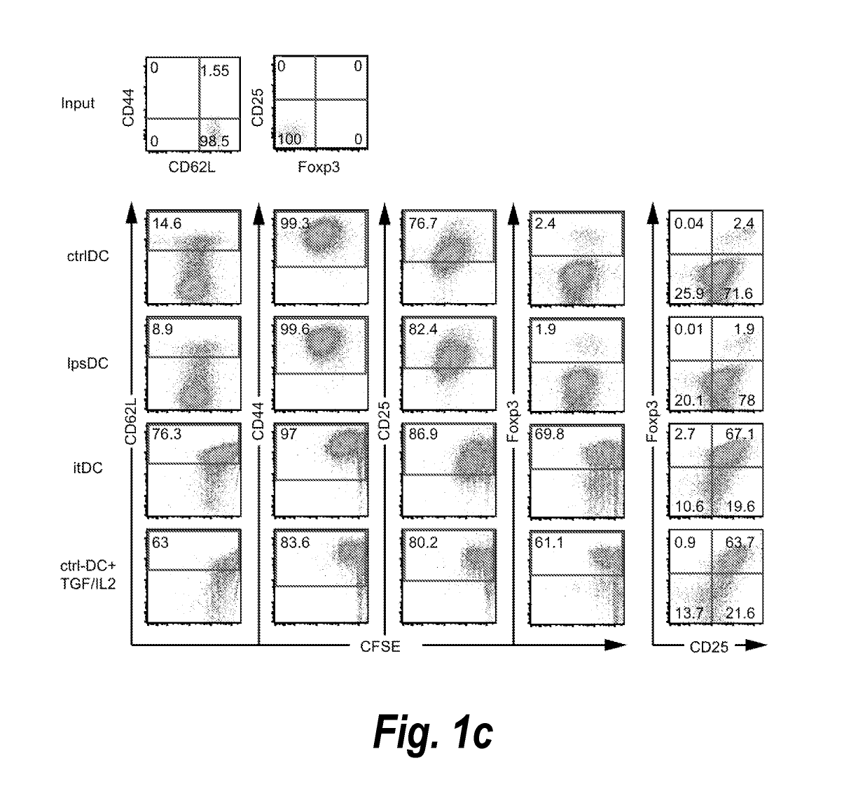

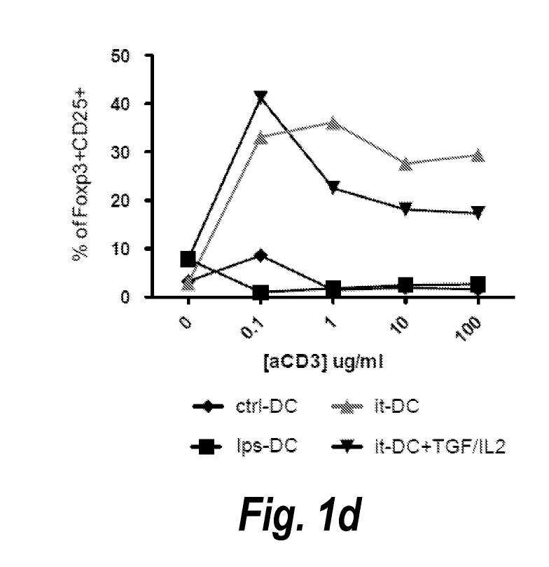

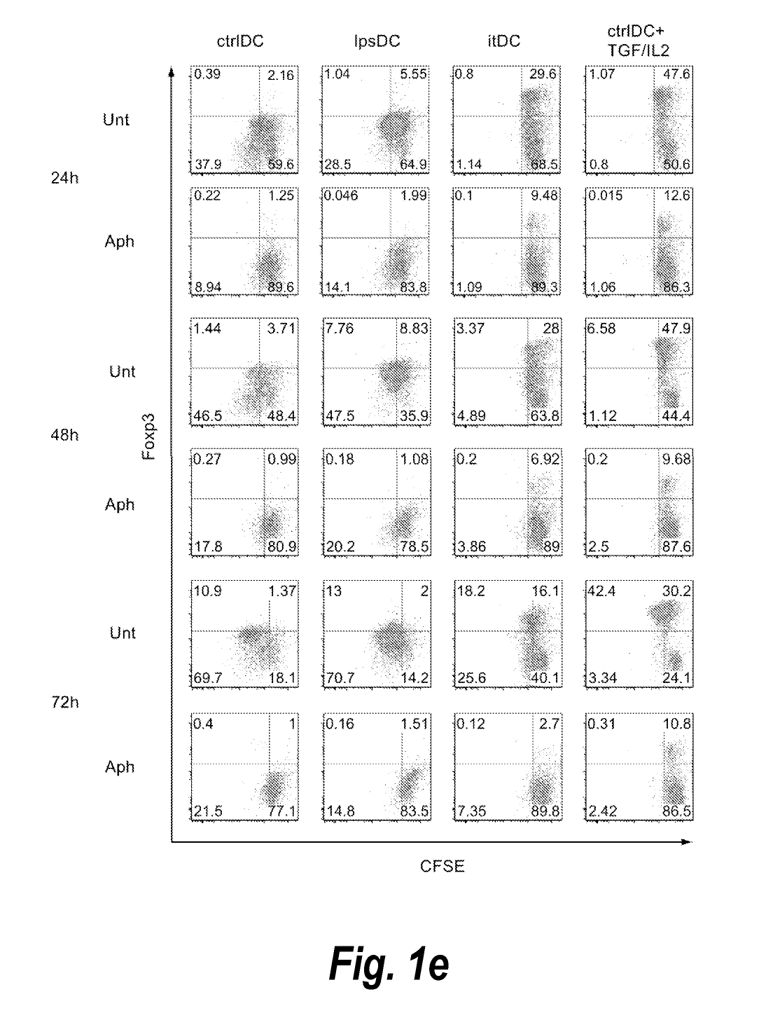

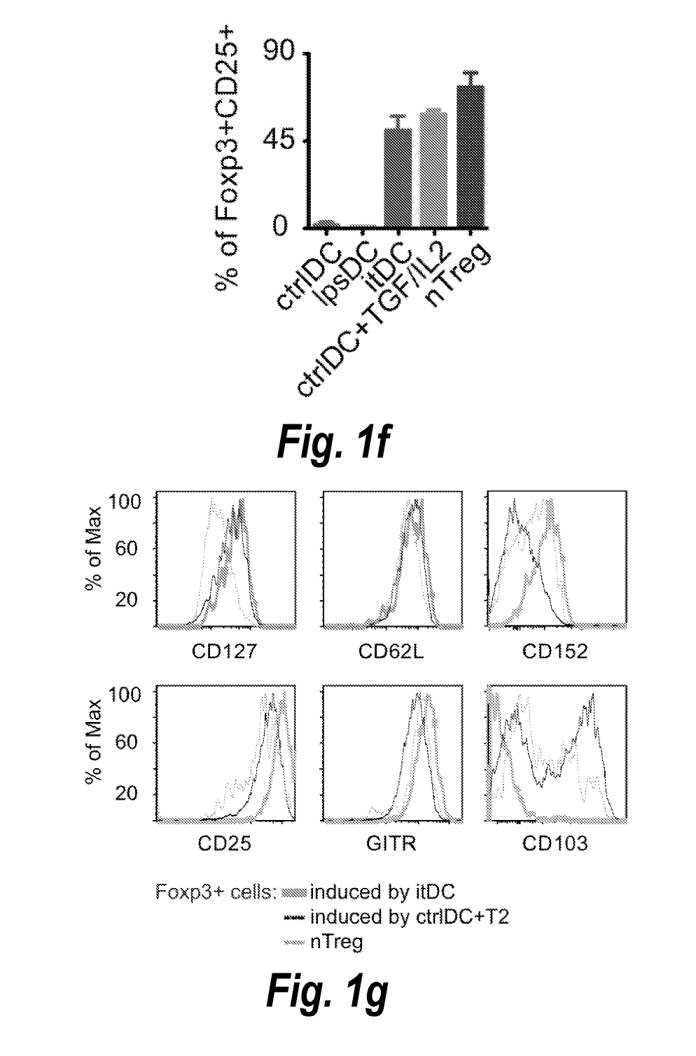

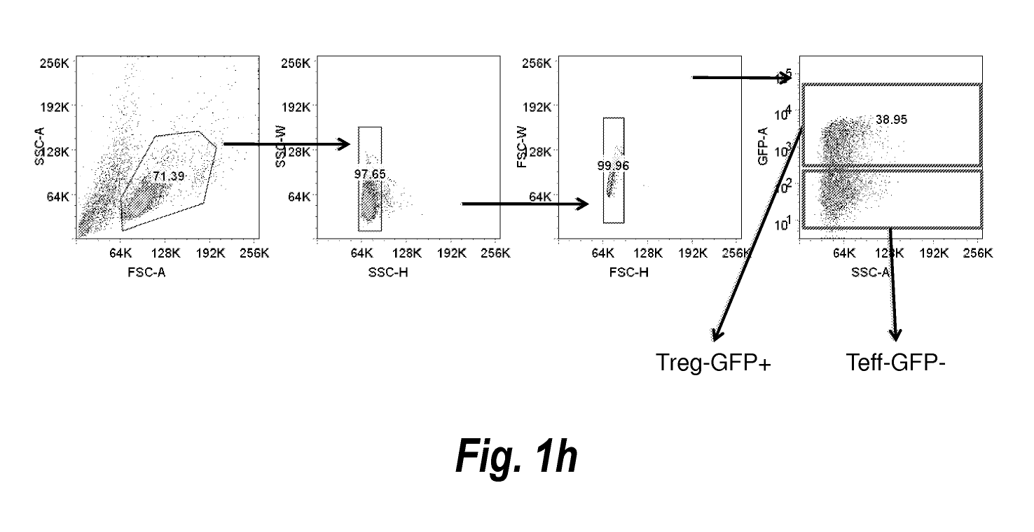

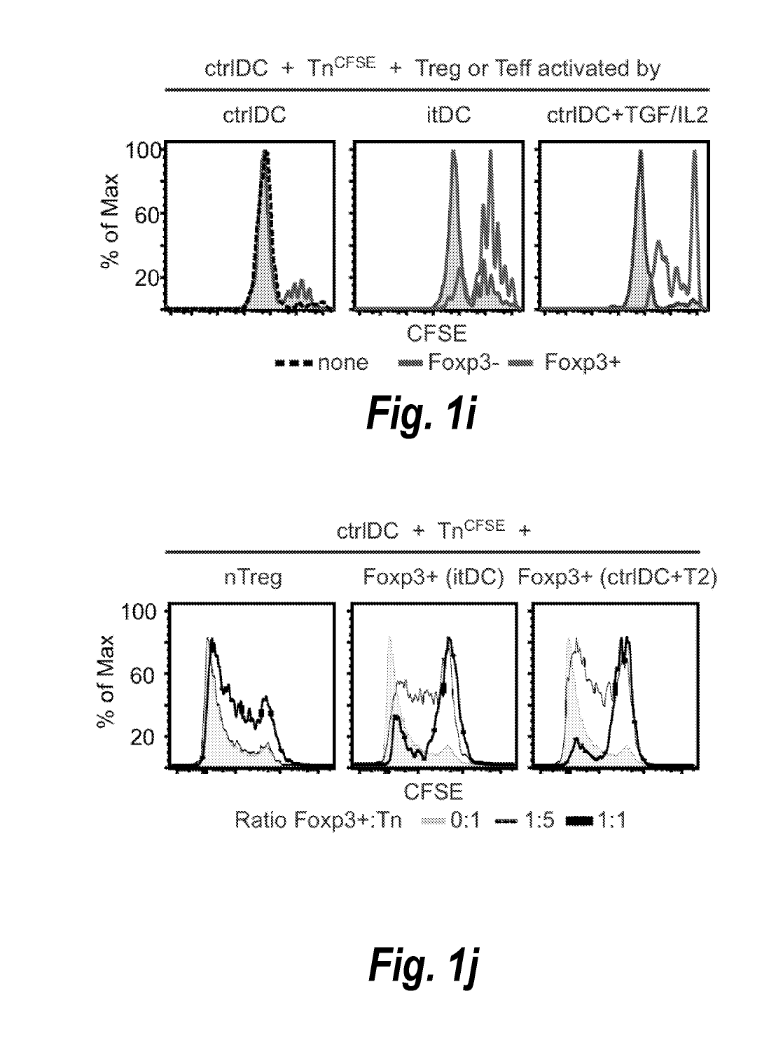

DC were incubated from 2 to 16 hours on ice or under tissue culture conditions (37° C., 5% CO2) with different combinations of Retinoic Acid (100 nM), Rapamycin (100 ng/ml) and TGFβ (2 ng/ml). Depending on the donor TCR transgenic strain for Tn, OVAp, OVA₃₂₃₋₃₃₉, MOG₃₅₋₅₅, VSV-G₄₁₅₋₄₃₃ (as described in materials and methods) were used in this incubation. Following treatment, DC were washed extensively (up to 6×). Tn were isolated from normal C57BL/6 animals, Foxp3GFP reporter animals, OTIIxFoxp3GFP, 2D2xFoxp3GFP or L7xFoxp3GFP transgenic x reporter mice by MACS and FACS sorting as previously indicated. In some conditions TGFβ and IL-2 (20 ng/ml both) were added to the T-DC coculture as a positive control (+TGF/IL2). When polyclonal Tn were used, anti-CD3 antibodies (1 μg/ml) served as activating stimulus in conjunction with DC. CD45.1+ or CD45.2+ animals were used as donors of DC and Tn (interchanged across experiments). This procedure is depicted schematically in FIG. 1a. FIG. 1b shows the percentages of Foxp3 and CD25 expressing cells identified by intracellular and surface staining or measuring GFP expression of CD4+CD11c− cells after 5 days in coculture with DC. In some experiments, 7AAD staining was used to identify dead cells. P values were calculated using a Bonferroni post-test of a regular two-way ANOVA test (*=p<0.05, **=p<0.01, ***=p<0.001). The results represent 5 different independent experiments, where each dot is one of triplicates. This screen of DC conditioning reveals that treatment of DC with a combination of rapamycin and TGFβ has significantly superior capacity to imprint DC with tolerogenic potential. This was assessed by measuring the Foxp3 expression on naïve T cells. Activation by cognate Ag or anti-CD3, but not cell division was required for Foxp3 induction in Tn (see below). Moreover, itDC induced Foxp3 in antigen-specific Tn whether they had been pulsed with a cognate peptide or whole protein.

c) Phenotype and Kinetics of Differentiation of Foxp3+ Tregs.

DC were isolated and purified from the spleens of CD45.1+ congenic animals as described previously. Untreated, control DC (ctrlDC), lipopolysaccharides-treated, immunogenic DC (lpsDC, 1 μg/ml), or rapamycin and TGFβ (exemplary inducers of itDC)(100 ng/ml and 20 ng/ml) were incubated for 2 hours under tissue culture conditions (37° C., 5% CO₂). Polyclonal Tn were isolated from CD45.2+ mice by MACS and FACS as described before, and were labeled with CFSE (see materials and methods) to assess their proliferative profile. In some instances, TGFβ and IL-2 (20 ng/ml) were added to the Tn-DC coculture as a positive control (ctrlDC+TGF/IL2). All Tn-DC cocultures were supplemented with 1 μg/ml of activating anti-CD3. FIG. 1c shows CD4+CD11c−CD42.1+ cells at day five of the culture after staining for the indicated markers (intracellular staining was perform to detect Foxp3). This figure shows representative plots of 3 independent experiments. These results demonstrate that in presence of itDC or the positive control ctrlDC+TGF/IL2, Tn acquire a phenotype similar to Treg displaying marked lower proliferation and high Foxp3 and CD25 expression when compared to ctrlDC and lpsDC.

d) Role of TCR Signaling Strength on Foxp3 Induction.

Cells were purified, cultured, harvested and analyzed under the same conditions as in Example 1c. Different concentrations of αCD3 antibody (2c11) were tested for their capacity to stimulate Tn and elicit Foxp3 expression. The proportion of Foxp3+CD25+ cells among single CD4+CD11c−7AAD− cells at 5 days after coculture is shown in FIG. 1d. Each point corresponds to the average of triplicates of one independent experiment, representative of two. This experiment shows that Foxp3 expression can be achieved by itDC even when strong TCR stimulation is provided.

e) Kinetics of Induction of Foxp3+Treg by Tolerogenic DC Ex Vivo.

Cells were purified, cultured, harvested and analyzed under the same conditions as in Example 1c in presence or absence of Aphidicolin (Aph) (4 μg/ml), a cell cycle blocker. FIG. 1e contains representative results of 2 independent experiments. Plots show single CD4+CD11c−CD45.1+ cells at different time points of the culture. These results show that induction of Foxp3 expression occurs within the original population of Tn, and that the output at day 5 represents in its majority Foxp3 upregulation and possibly expansion of these Foxp3+ T cells.

f, g) Phenotype of Treg vs nTreg.

Cells were purified, cultured, harvested and analyzed under the same conditions as in Example 1c. Tn and natural-occurring Treg (nTreg) were isolated from Foxp3GFPxCD45.1 reporter x congenic mice by MACS and FACS sorting of Foxp3GFP− Tn or pre-existing natural occurring Foxp3GFP+ Treg (CD4+CD62Lhigh CD44low CD25+Foxp3GFP+, nTreg) and activated by different types of DC in presence of activating anti-CD3. After 5 days in culture, the phenotype of Treg induced by itDC or the positive control (TGFβ and IL-2) was compared to the activated nTreg (with ctrlDC). FIG. 1f depicts the percentage of Foxp3+CD25+ cells following coculture with the indicated DC populations among CD4+CD11c−CD45.1+7AAD− cells (representative results of 3 independent experiments). Plots shown in FIG. 1g depict the expression of the indicated makers on DC-instructed Treg or nTreg (Foxp3+CD25+ CD4+CD45.1+7AAD− cells). With the exception of CD103, itDC-stimulated Treg display a very similar expression of GITR, CD152, CD25, CD127, CD62L when compared to nTreg.

h) FACS Sorting Strategy for Foxp3+ Cells.

DC from CD45.1+ animals were incubated from 2 hours under tissue culture conditions (37° C., 5% CO2) with just tissue culture media (ctrlDC), rapamycin (100 ng/ml) and TGFβ (2 ng/ml)(itDC), LPS (1 μg/ml, lpsDC) and OVA₃₂₃₋₃₃₉ (as described in materials and methods). Following treatment, DC were washed 3 times. Tn were isolated from CD45.2xOTIIxFoxp3GFP animals, by MACS and FACS sorting as previously indicated. In some conditions TGFβ and IL-2 (20 ng/ml both) were added to the T-DC coculture as a positive control (ctrlDC+TGF/IL2). After 5 days in culture with itDC or ctrlDC+TGFβ/IL-2, T cells are distributed in two major populations according to the expression of GFP. These cells were FACS-sorted and used to test their suppressive capacity over the proliferation of Tn.

i) Foxp3+ but not Foxp3− Cells Suppress.

GFP+ Treg and GFP− activated effector T cells (Teff) were obtained as described in Example 1h. A second round of CD45.1+DC equally loaded with OVAp and OTII+CD45.1+ Tn were sorted and stained with CFSE (Tn^{CFSE}) and used as readout or control for the suppression assay, respectively. These freshly isolated CD45.1+ Tn were cultured with differentiated CD45.2+ Foxp3GFP+ Treg and Foxp3GFP− Teff cells as indicated in FIG. 1i. The CFSE profile of Tn^{CFSE} was assessed by FACS (CD4+Vα2+CD45.1+CD11c−7AAD− cells). These results, depicted in FIG. 1i, demonstrate that T cells expressing Foxp3 (identified using the reporter GFP) after stimulation with itDC have suppressive activity over Tn ex vivo. Freshly isolated Tn (not shown) or Teff cells from the same culture that do not express Foxp3 do not have this suppressive activity.

j) Suppressive Function of Tregs Depends on the Tn to Treg Ratio.

CD45.2+Foxp3GFP+ Treg were isolated as described above and compared to freshly isolated CD45.2+Foxp3GFP+ nTreg for their suppressive capacity at different ratios of suppressor Treg versus responder CD45.1+Tn^{CFSE}. FIG. 1j shows representative plots of 2 experiments for CD45.1+CD4+TCRVα2+CD11c−7AAD− T cells at day four of co-culture. These results show that the suppressive activity of Foxp3+ Treg stimulated by itDC is comparable to nTreg and depends, in part, on the ratio of Treg versus Tn cells.

### Example 2

### Human Induced Tolerogenic Monocyte-Derived Dendritic Cells (itMoDC)

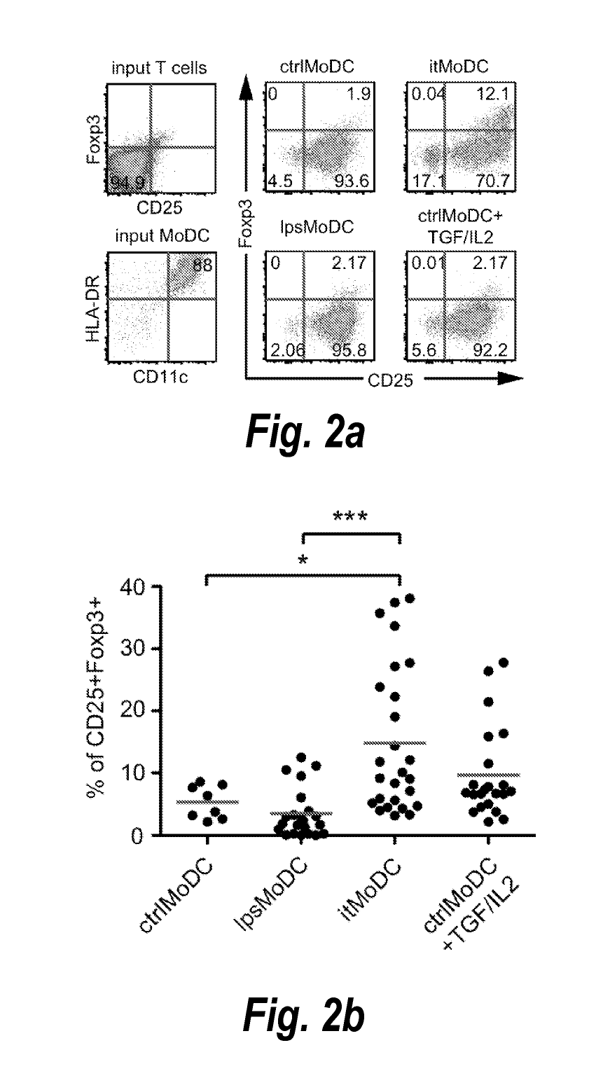

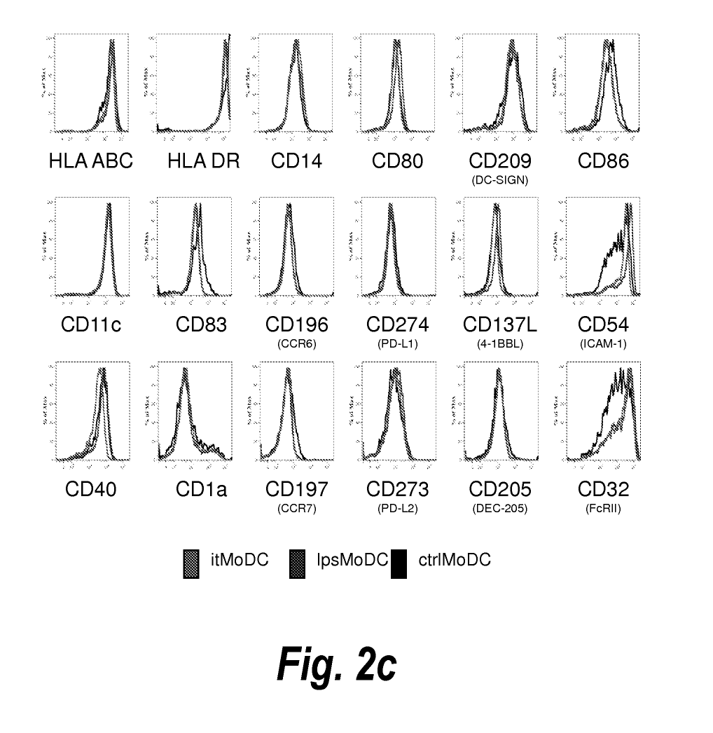

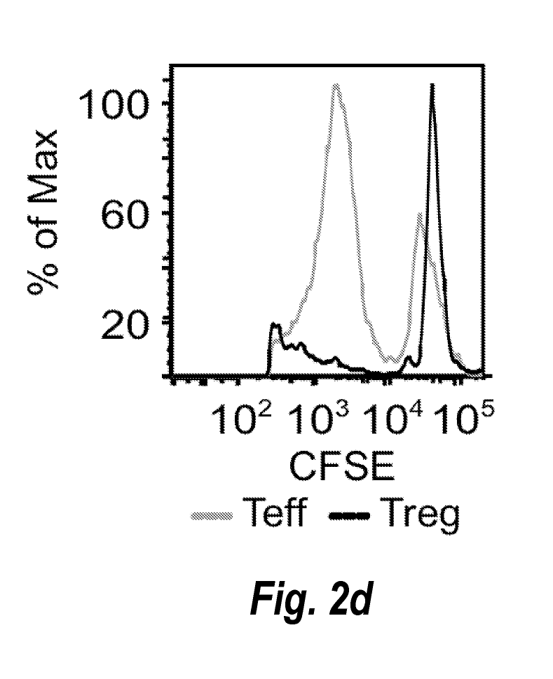

Monocytes and naïve CD4+ T cells were purified from peripheral blood mononuclear cells (PBMC) of different donors by magnetic negative selection. Monocytes were differentiated into DC using standard protocols as set forth above (Monocytes were purified from PBMC by negative selection using magnetic negative selection by MACS and cultured for 6 days in presence of GM-CSF (100 ng/ml) and IL-4 (15 ng/ml), combined with the indicated treatments, during their differentiation (see Materials and Methods). FIG. 2a shows the input Tn cells and MoDC (left panels), and the expression profile of Foxp3 and CD25 of T cells after activation with MoDC treated as indicated (ctrl—untreated; it—rapamycin/TGFβ; lps—lipopolysaccharides). FIG. 2b shows compiled data from 5 independent different experiments. Each dot represents a different Tn cell donor (average of triplicates). MoDC and Tn were differentiated from 5 and 21 different donors, respectively. P values were calculated using a Bonferroni post-test of a regular two-way ANOVA test (*=p<0.05, **=p<0.01, ***=p<0.001). These experiments show that treatment of human DC with rapamycin and TGFβ elicit Foxp3-inducing capacities in human Tn that is significantly different from other DC. Surprisingly the phenotype of these different subsets of DC regarding a panel of costimulatory molecules was very similar as shown in FIG. 2c. In order to test whether suppressive function was induced by itMoDC, activated T cells (CD45RA−CD25+/−, Teff) and putative Treg (CD45RA−CD25high) were FACS sorted and added to cocultures of MoDC and fresh CFSE-labeled Tn with anti-CD3. The histograms in FIG. 2d show CFSE dilution on Tn (proliferation), after 3-day culture with CD25− (Teff) or CD25high (Treg). Therefore, human itDC-induced Foxp3+ T cells suppressed proliferation of cocultured Tn.

### Example 3

### Phenotype of itDC

The phenotype of itDC for their expression of presentation and costimulatory molecules as well as the composition in subsets was surveyed. No significant differences could be observed between ctrlDC and itDC whether they were freshly isolated or stimulated with toll-like receptor (TLR) agonists with respect to expression of maturation phenotype (presentation and costimulatory molecules) or function (induction of Foxp3). By contrast, significant differences exist between DC subsets in their basal capacity to induce Foxp3 in Tn. However, all DC subsets respond similarly to rapamycin and TGFβ treatment (fold increase of Foxp3-inducing capacities). A molecular analysis revealed that itDC had an inactive mammalian target of rapamycin (mTOR) pathway. DC obtained by differentiation of bone marrow progenitors (BMDC) using GM-CSF and IL-4 yielded poor tolerogenic function when treated with rapamycin and TGFβ. However differentiation in presence of FLT3L followed by rapamycin and TGFβ treatment induced BMDC with the capacity to induce Foxp3 expression on Tn.

a) Expression of Presentation and Costimulatory Molecules.

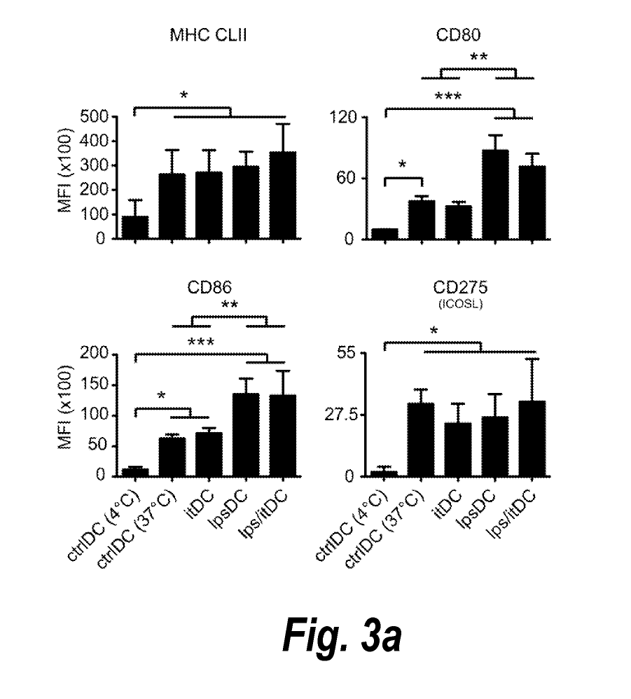

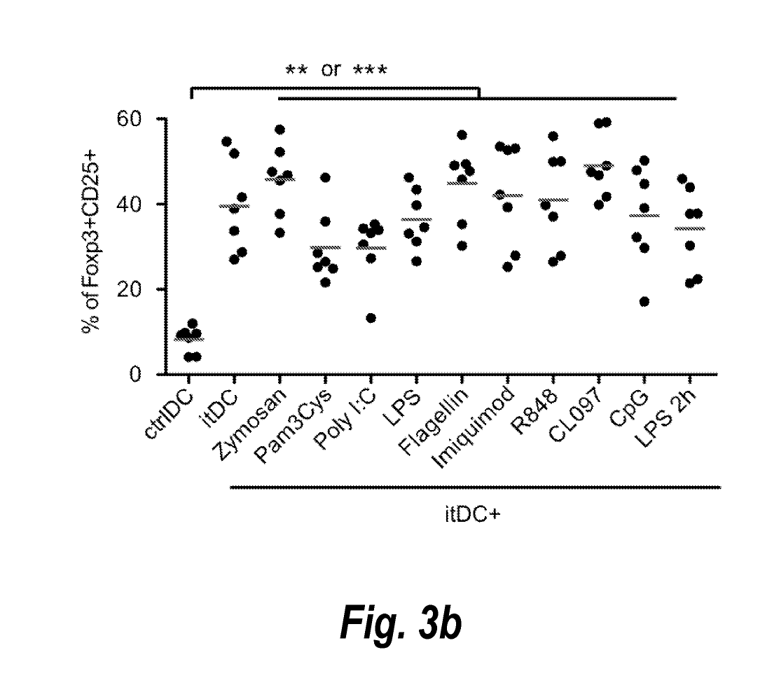

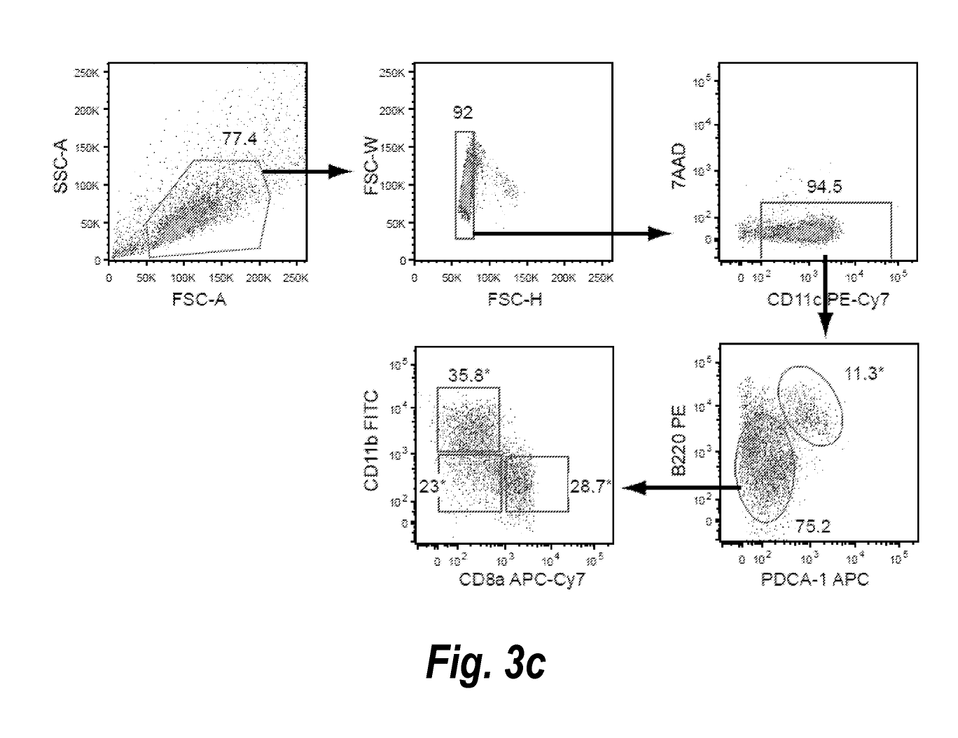

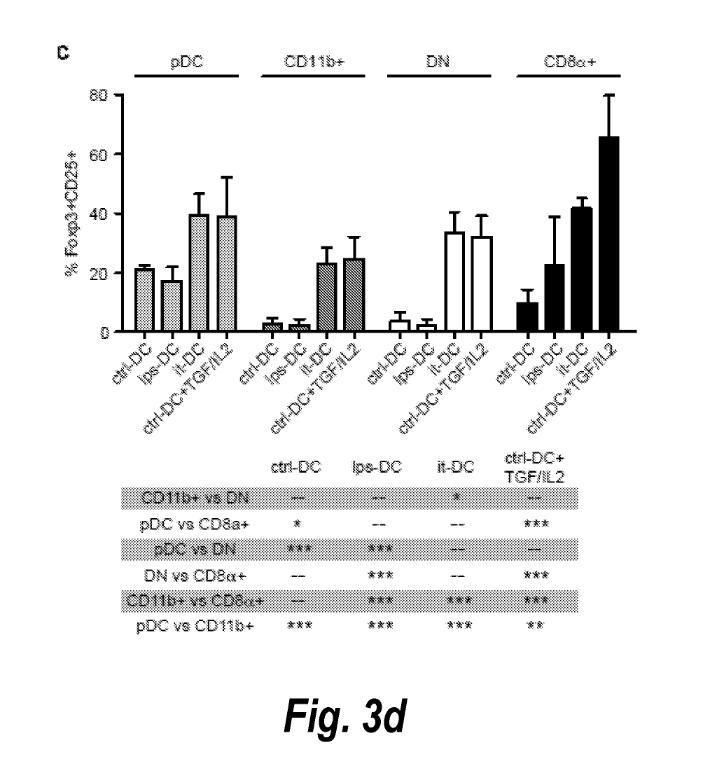

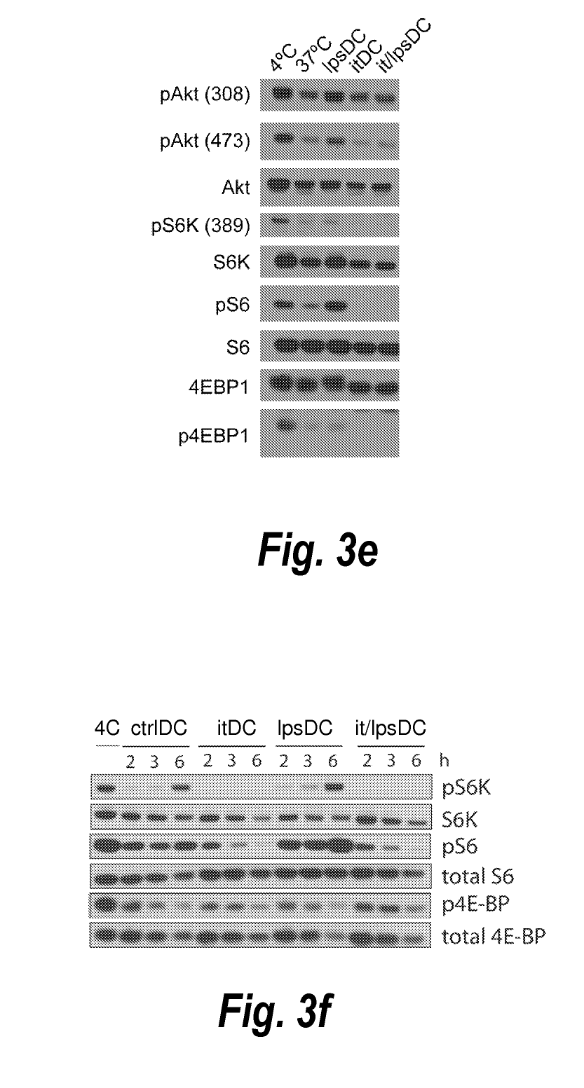

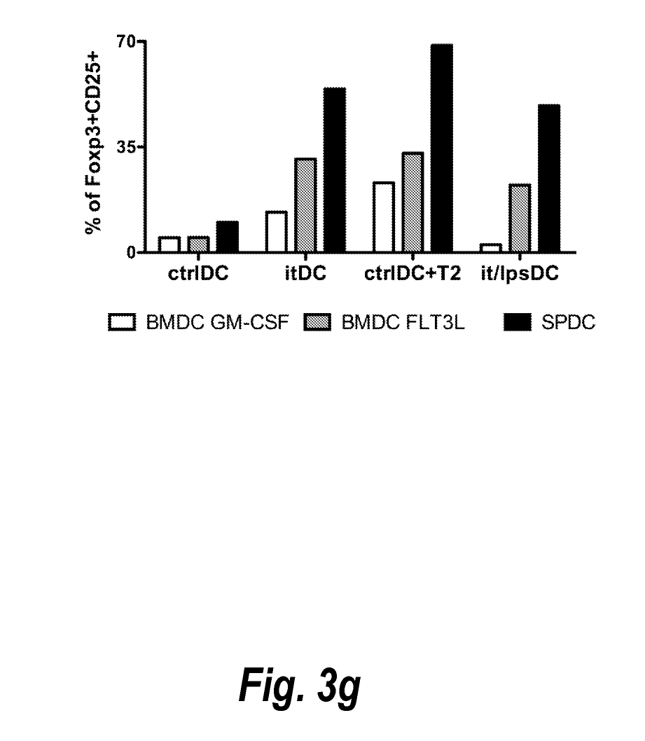

Mouse DC were purified and treated as described in Example 1 h but were incubated overnight in regular tissue culture conditions. Cells were harvested stained as indicated in FIG. 3a. Shown are the average (+/−standard deviation) of the mean fluorescence intensities of the indicated markers on CD11c+7AAD− cells (results are average of triplicates and representative of three independent experiments). P values were calculated using a Bonferroni post-test of a regular two-way ANOVA test. These results demonstrate that the phenotype of itDC is comparable to their control counterpart with respect to expression of major histocompatibility complex CLII expression and the costimulatory molecules CD80, CD86 and ICOSL. Similarly the levels of expression of CD40, CD134L (OX40L), CD137L (4-1BBL), CD273 (PD-L2) and CD274 (PD-L1), CD276 (B7-H3), B7-H4, ICAM1-3, LFA-1, α4β7 integrin, CCR2, CCR4, CCR5, CD196 (CCR6), CD197 (CCR7), CCR9, CX3CR1, CXCR3 and CXCR4 were not distinguishable between itDC and ctrlDC (before or after maturation with LPS, not shown). Importantly, itDC seem to be capable of acquiring high levels of presentation and costimulatory molecules in presence of TLR agonist suggesting that they are capable of acquiring a mature phenotype.

b) Function of Mature and Immature itDC.

DC were isolated from normal animals, treated, loaded with OVAp and used to stimulate Tn from OTIIxFoxp3GFP animals as described in Example 1h. When indicated, toll-like receptor agonists were added simultaneously with rapamycin and TGFβ (itDC+). In one condition LPS was added 2 hours before the 2 hour treatment with rapamycin and TGFβ treatment (LPS 2 h). Exposure of itDC to multiple TLR agonists before or during rapamycin and TGFβ treatment did not alter tolerogenicity (FIG. 3b), despite upregulation of maturation markers (see above).

c) Sorting Strategy for DC Subsets.

In order to test the capacity of different DC subsets to induce Foxp3 expression on Tn, DC were isolated from animals inoculated with melanoma-producing FLT3L and FACS sorted to obtain CD11c+B220+PDCA1+(pDC) and CD11c+B220−PDCA1−: CD11b+CD8α− (CD11b+), CD11b-CD8α+(CD8α+), and CD11b−CD8α− (DN) using the strategy depicted here in FIG. 3c.

d) Comparison of the Relative Tolerogenicity of DC Subsets.

DC subsets were treated and sorted as described above (or vice versa) and used to activate OTII+Foxp3GFP− cells as in Example 3b. Analysis of their capacity to induce Foxp3 in Tn (FIG. 3d) revealed that even untreated DC have a characteristic baseline capacity to induce Treg, with pDC≧CD8α⁺>>CD11b⁺=CD8α⁻CD11b− (DN). However, when treated with rapamycin and TGFβ, the tolerogenicity of each subset was substantially enhanced, indicating that all DC are susceptible to this conditioning.

The results in FIGS. 3b and 3d are represented as percentages of Foxp3GFP+CD25+CD4+CD11c−7AAD− cells at day five of the culture. All p values were calculated using a Bonferroni post-test of a regular one- or two-way ANOVA test.

e) Status of the mTOR Pathway in itDC.

DC were isolated and treated as in previous figures to obtain itDC, lpsDC and ctrlDC. An additional control, freshly isolated DC kept on ice (4° C.), was included. After 6 hours the cells were lysed and analyzed by western blot for the species indicated (left). The results in FIG. 3e show that, as expected, treatment with LPS increases the activation of the mTOR pathway as evidenced by the phosphorylation of the downstream targets of mTOR S6, S6K and 4EBP1. Basal and increased phosphorylation of these factors was completely blocked by treatment with rapamycin and TGFβ. Very little to no effect can be observed on the activation of Akt.

f) Status of the mTOR Pathway in itDC: Kinetics.

DC were isolated and treated as in previous figures to obtain itDC, lpsDC and ctrlDC. An additional control, freshly isolated DC kept on ice (4° C.), was included. At different time points the cells were lysed and analyzed by western blot for the species indicated (left). The results in FIG. 3f show that, as expected, treatment with LPS increases the activation of the mTOR pathway as evidenced by the phosphorylation of the downstream targets of mTOR S6, S6K and 4EBP1. Basal and increased phosphorylation of these factors was completely blocked by treatment with rapamycin and TGF3.

g) Comparison Between DC of Different Origins:

Bone marrow cells were cultured for 8 days in the presence of GM-CSF and IL-4 or FLT3L following standard protocols to obtain bone marrow derived DC (BMDC, see materials and methods). Some of these cells were treated at day 6 for another two days with rapamycin (5 ug/ml) and TGFβ (2 ng/ml) to obtain itDC or combined with lipopolysaccharide from E. coli (it/lpsDC, 1 ug/ml). All cells were fed with OVAp 24 hours before harvesting to activate OTII+Foxp3GFP−CD45.1+Tn. After 5 days in this co-culture the presence of CD25+Foxp3+ among Vα2+CD4+CD45.1+ cells was assessed by FACS. Positive controls were cell cultures in which additional TGFβ and IL-2 were added (ctrlDC+T2). These results show that BMDC obtained from BM progenitors in presence of FLT3L have a similar capacity to induce Foxp3 on Tn to splenic DC while BMDC derived in presence of GM-CSF do not.

### Example 4

### Mechanisms of Tolerance Induction by itDC

The involvement of several tolerogenic factors in the capacity of itDC to induce Foxp3 expression on Tn was assessed by obtaining DC from animals deficient for their expression or using blocking antibodies. The results show that itDC function is independent of the production of IL-6, IL-10, TGFβ, FasL TSC1, EBI3 and IDO1 by DC.

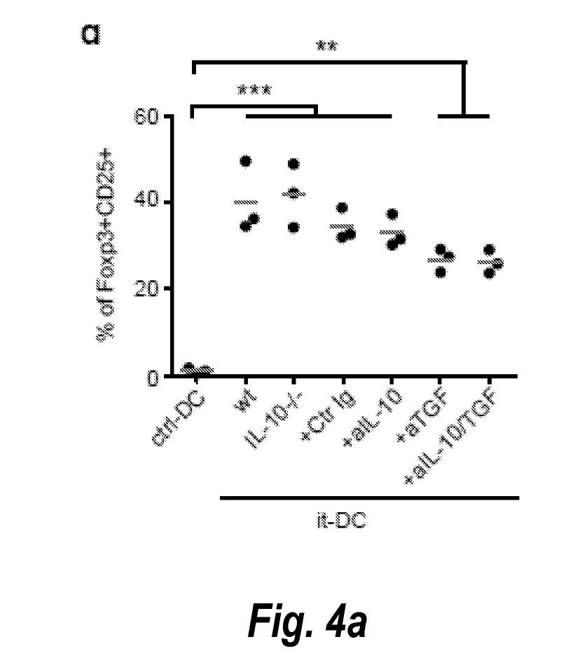

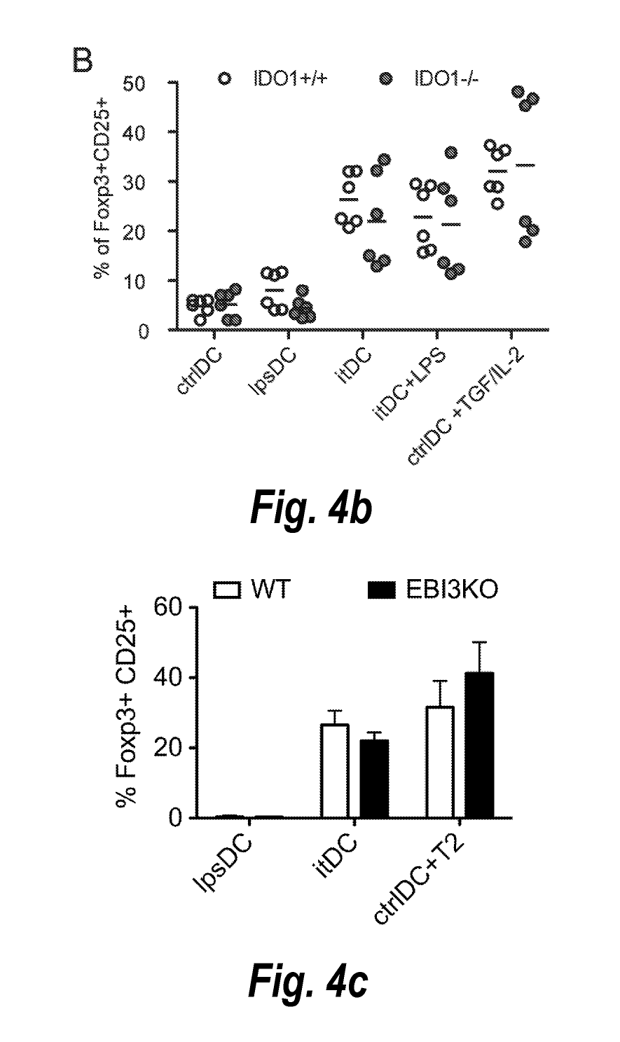

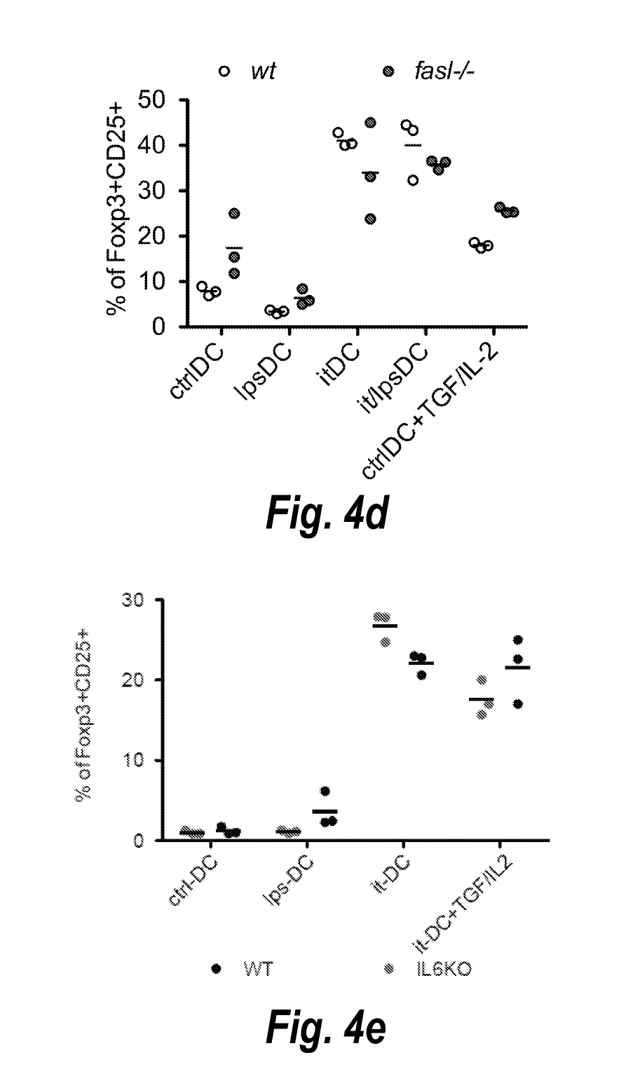

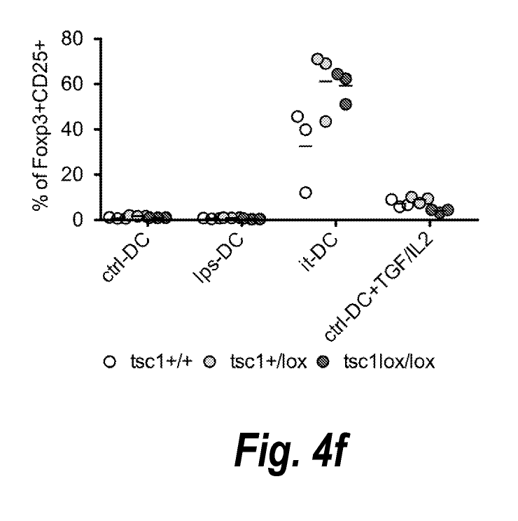

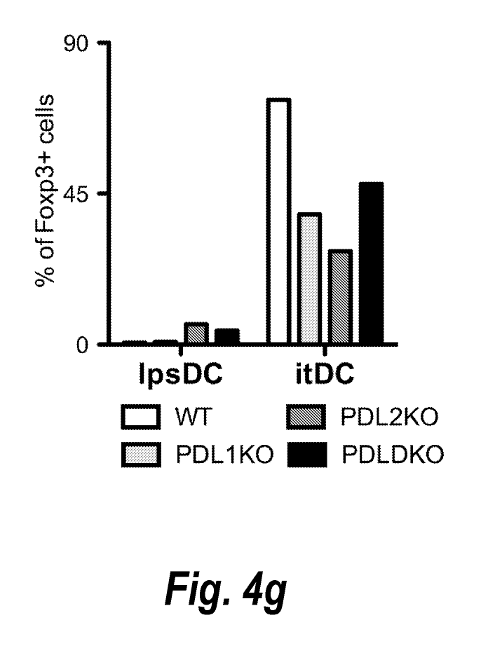

Similar cultures as described previously in Example 1h containing different types of OVAp-loaded DC and OTII+Foxp3GFP− Tn were performed adding 10 μg/ml of blocking anti-IL-10 (JES5-16E3) and anti-IL10R (1B1.3a) (+aIL-10) and/or blocking anti-TGFβ (1D11, aTGF) or isotype controls (+Ctrl Ig). In one condition, IL-10 deficient (−/−) animals were used as donor of DC. Results are shown in FIG. 4a, where each dot represents an independent experiment and an average of triplicates. These results demonstrate that stimulation of Foxp3 in Tn cells does not involve secretion of IL-10 or TGFβ by itDC.

In addition, when IDO1 (FIG. 4b), EBI3 (FIG. 4c), FasL (FIG. 4d), IL-6 (FIG. 4e) sufficient or deficient DCs were conditioned as above, no significant difference was observed between the groups. Similarly, when TSC1, a factor controlling mTOR activity was deleted by crossing CD11c-Cre transgenic animals to TSCllox/lox conditional knock out animals to obtain animals in which all DC lack TSC1 expression (tscllox/lox), express only one functional allele (tsc1+/lox) or both (tsc1+/+) no difference in the capacity of itDC to induce Foxp3 on Tn was observed (FIG. 4f). All animals were littermates. However when DC from animals lacking the expression of the programmed death 1 receptors (PD1), PDL1, PDL2 or both were used, a reduction on the capacity to evoke Foxp3 expression was observed (FIG. 4g),

These experiments show that itDC induce Foxp3 expression on Tn partially trough the PD1 pathway independently of IL-6, IL-10, TGFβ, FasL TSC1, EBI3 and IDO1.

### Example 5

### Effects of itDC on T Cells Ex Vivo are Contact-Dependent and Influenced by the itDC:T Cell Ratio

To better characterize the effects of itDC on Tn during their coculture ex vivo, similar experiments to those in Example 1h were performed using transwell experiments or combining itDC with immunogenic lpsDC to determine if T cell survival is affected in these cocultures or whether cell contact was required for the induction of Foxp3 on Tn by itDC.

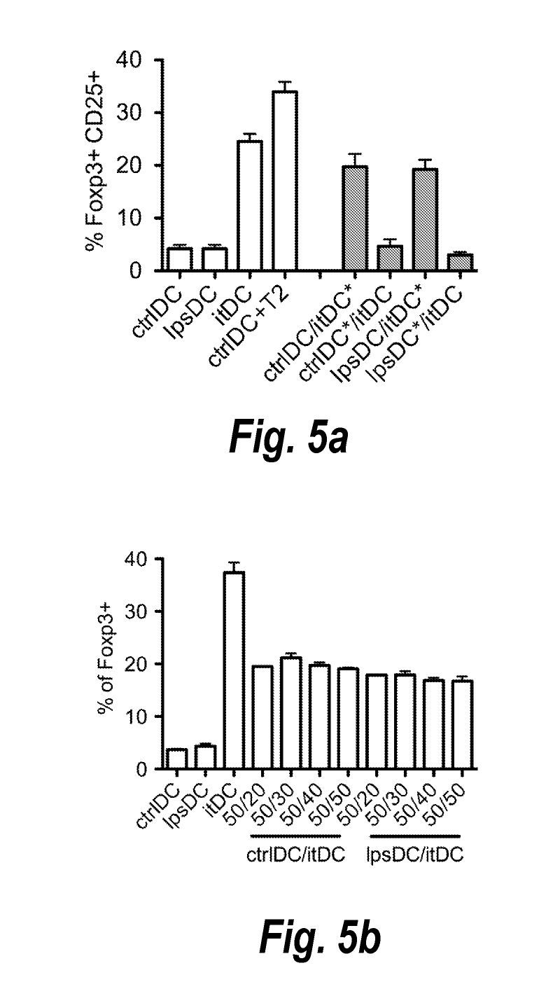

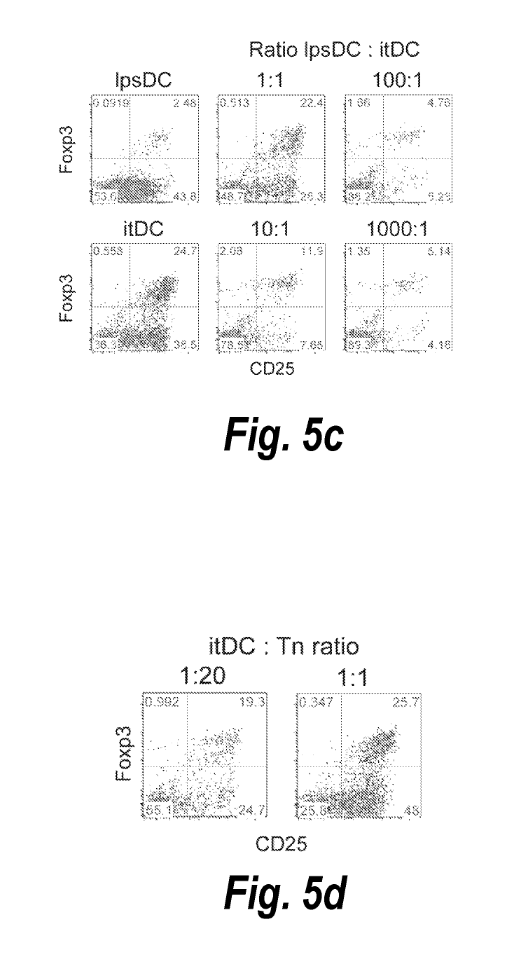

In FIG. 5a DC from normal CD45.2+ animals were isolated, loaded with OVAp, treated as in previous experiments to produce ctrlDC, lpsDC and itDC and used to activate OTII+Foxp3GFP−CD45.1+ Tn in transwell chambers. DC were loaded alone or in combination with Tn (*) in the upper/lower chamber. The presence of CD25+Foxp3+ among Vα2+CD4+CD45.1+ cells was assessed 5 days later by FACS. These experiments show that direct contact is necessary between itDC and Tn to achieve upregulation of Foxp3 expression. Further, the presence of immunogenic lpsDC did not prevent itDC to induce Foxp3 expression. Indeed, in FIG. 5b similar cocultures of itDC and Tn as above were performed but different combinations and ratios of DC were loaded as indicated. The presence of CD25+Foxp3+ among Vα2+CD4+CD45.1+ cells was assessed 5 days later by FACS. There were no detectable differences at ratios of 50 itDC to 50 ctrlDC or lpsDC or lower. Interestingly the proportions of Treg induced are lower when using half of the itDC in these experiments. However, as little as 1 itDC for every 100 lpsDC were able to induce Foxp3+ expression on Tn (FIG. 5c) suggesting that itDC can evoke tolerogenic events even in presence of a majority of fully immunogenic DC. The tolerogenic effect of itDC is dependent on the itDC:Tn ratio as the proportions of Foxp3+Treg can be decreased (FIG. 5c) or increased (FIG. 5d) when adding less or more itDC, respectively.

### Example 6

### Depletion of Antigen-Specific T Cells and Induction of Foxp3 Expression by itDC Ex Vivo

To further characterize the effect of itDC on naïve (Tn) and effector T cells (Teff), DC were loaded with OVAp and differentially conditioned as described previously to evoke an immunogenic (ctrlDC<lpsDC) or tolerogenic (itDC) phenotype.

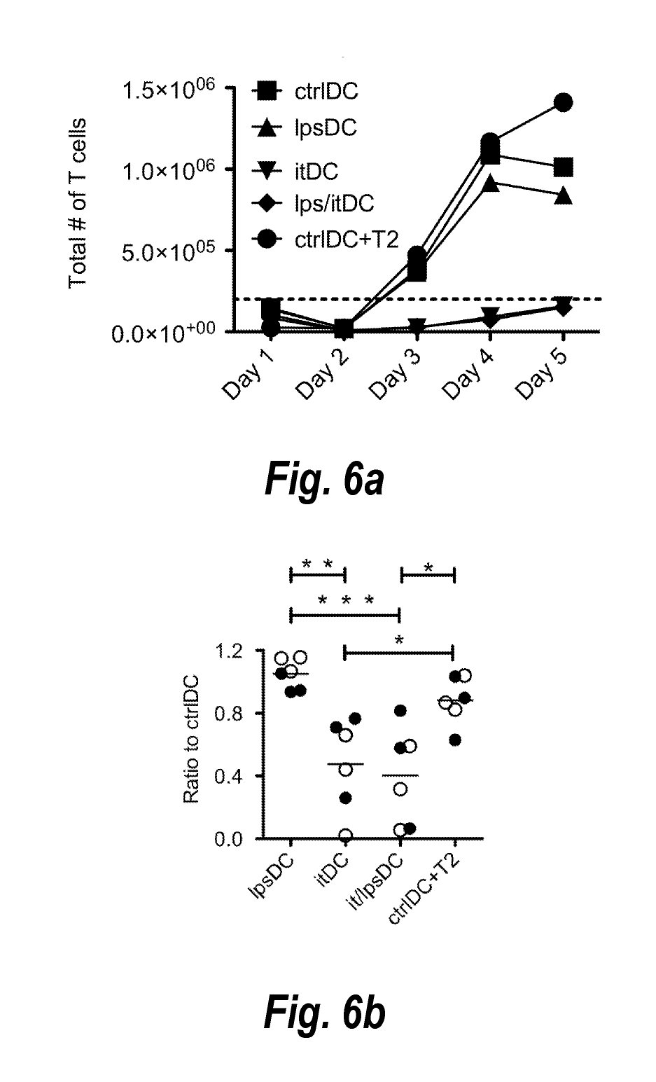

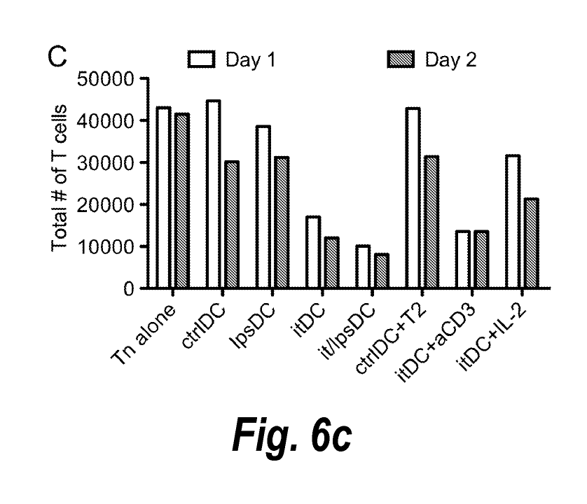

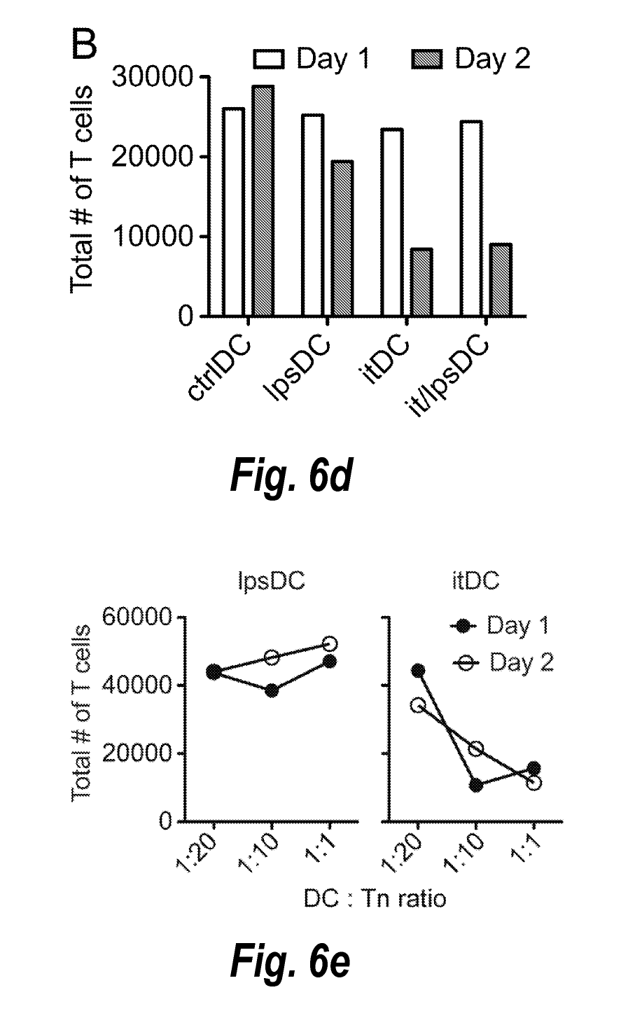

DC were used to activate OVA-specific OTII+Tn. Robust T cell proliferation started on day 3 in cocultures containing lpsDC and ctrlDC and was also apparent when TGFβ and IL-2 were added to ctrlDC (ctrlDC+T2; FIG. 6a). By contrast, Tn that were exposed to itDC proliferated poorly. Interestingly, T cell numbers also underwent dynamic changes prior to day 3; irrespective of the DC subset used, there was a marked contraction of Tn within the first two days. A careful comparison of T cell numbers during days 1 and 2 revealed that the presence of itDC resulted in a ˜2-fold greater loss of Tn when compared to ctrlDC (normalized to ctrlDC, FIG. 6b). Furthermore, when compared to the number of Tn that survived when cells were left in culture alone, the presence of ctrlDC and lpsDC had no effect, whereas itDC and LPS-activated itDC (it/lpsDC) caused a dramatic additional loss of T cells (FIG. 6c). This effect of itDC was not reversible by concomitant strong stimulation with plate-bounded activating aCD3 plus aCD28, whereas additional IL-2 partially restored T cells numbers (FIG. 6c). Importantly, profound T cell depletion was also observed when activated Teff were cocultured with itDC. However, Teff numbers were only affected at day 2 (FIG. 6d).

Further experiments revealed that the T cell depleting effect of itDC on Tn is dependent on the T cell:itDC ratio (FIG. 6e). At the lowest ratio (1:1, which reflects 10-times greater DC numbers than in our standard assay) viable Tn were reduced by >75% (FIG. 4d). By contrast, the capacity of itDC to induce Foxp3 in Tn was only minimally affected by changes in Tn:itDC ratio (Example 5b and 5d), suggesting that the mechanisms by which itDC delete conventional T cells and induce Treg may be distinct.

FIG. 6a) Kinetics of Tn Survival Ex Vivo.

DC were loaded with OVA protein and treated with 50 pg/ml rapamycin and 20 ng/ml TGFβ (R+T), or LPS (1 μg/ml) and washed to obtain itDC and lpsDC, respectively. Some DC were co-treated with R+T and LPS (it/lpsDC). Control DC (ctrlDC) were identically cultured with only the solvents used for each reagent. As a positive control, TGFβ and IL-2 were added to the Tn-ctrlDC cocultures (ctrlDC+T2) to directly induce Foxp3 (FIG. 6a). The broken line in represents the input of Tn.

FIG. 6b) Selective Early Disappearance of Tn Induced by itDC.

Data in the previous figure were normalized to T cell numbers in cocultures with ctrlDC. White symbols represent results obtained at day 1 whereas filled symbols represent day 2 ratios.

FIG. 6c) TCR Stimulation does not Overcome the Deleterious Effect of itDC.

OTII+Foxp3GFP− Tn were stimulated as in the previous figures by different types of OVAp-loaded DC (FIG. 6c). Tn were left alone (Tn alone), or mixed with different samples of DC as indicated. In one condition, activating aCD3 and aCD28 MAbs were plate-bound to maximally activate Tn in the presence of itDC (itDC+aCD3). In another condition, supplemental IL-2 was added to coculture starting at day 0 (itDC+IL-2).

FIG. 6d) itDC Reduce Teff Cell Numbers.

Similar experiments as before were performed using Tn that were activated for 5 days in culture with lpsDC and subsequently cocultured in the presence of indicated DC subsets.

FIG. 6e) itDC:T Ratio Affects T Cell Disappearance.

To assess whether T cell depletion by itDC depends on the number of itDC present in the culture, Tn were cocultured with different numbers of OVAp-pulsed itDC. The figure shows the total number of Tn on day 1 and day 2. The ratio in standard assay employs 1 DC to 10 T cells (1×10⁴ DC and 1×10⁵ Tn).

Shown in FIGS. 6a, 6c, 6d and 6e are TCRβ+CD11c−Vα2+7AAD− cell counts.

### Example 7

### Depletion of Antigen-Specific T Cells and Induction of Foxp3 Expression by itDC In Vivo

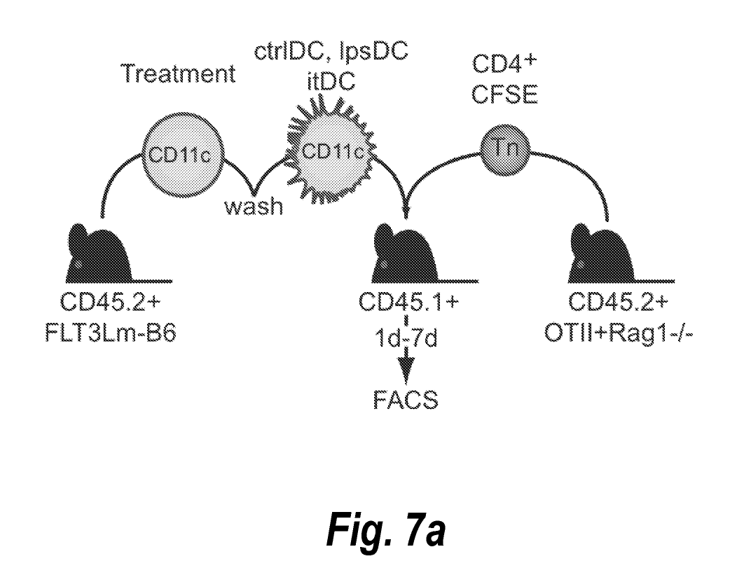

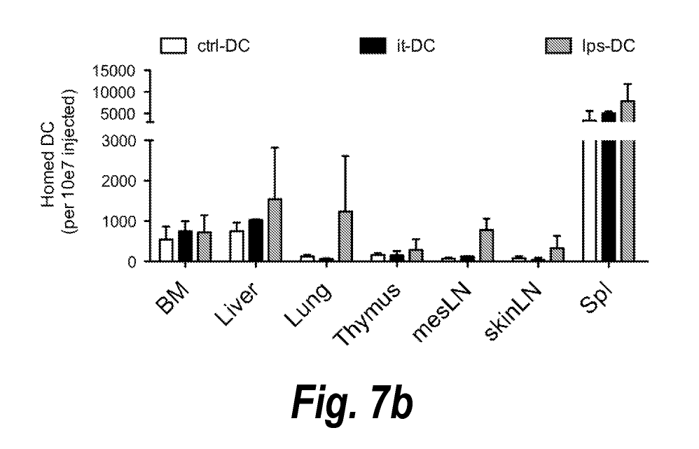

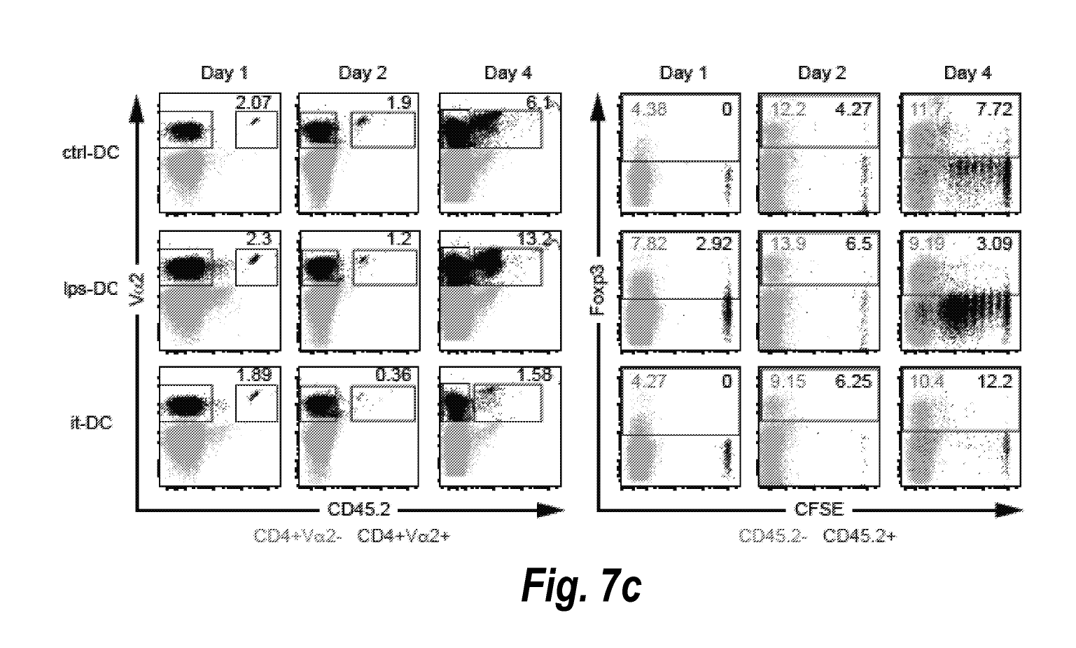

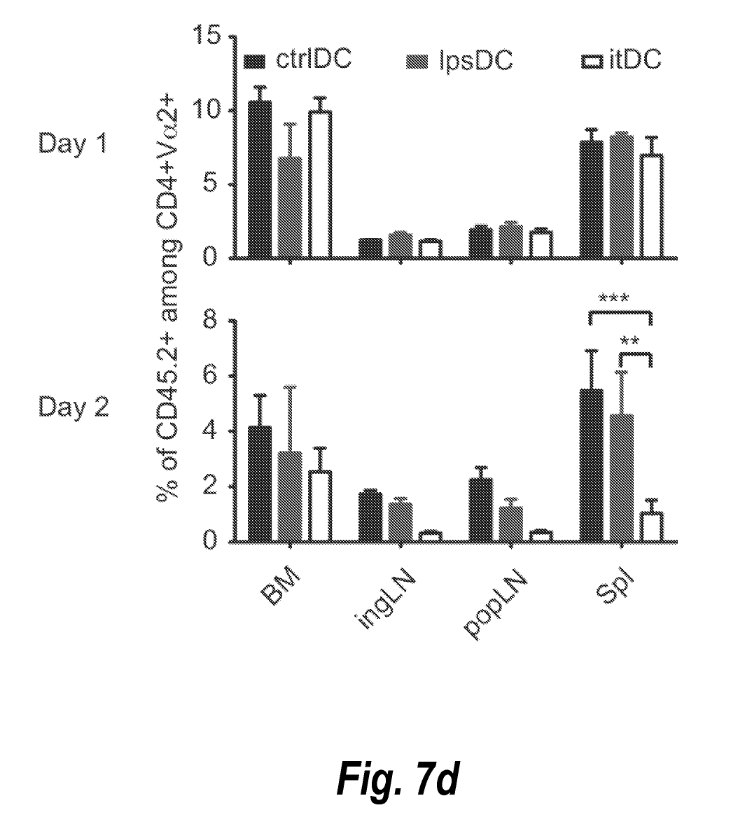

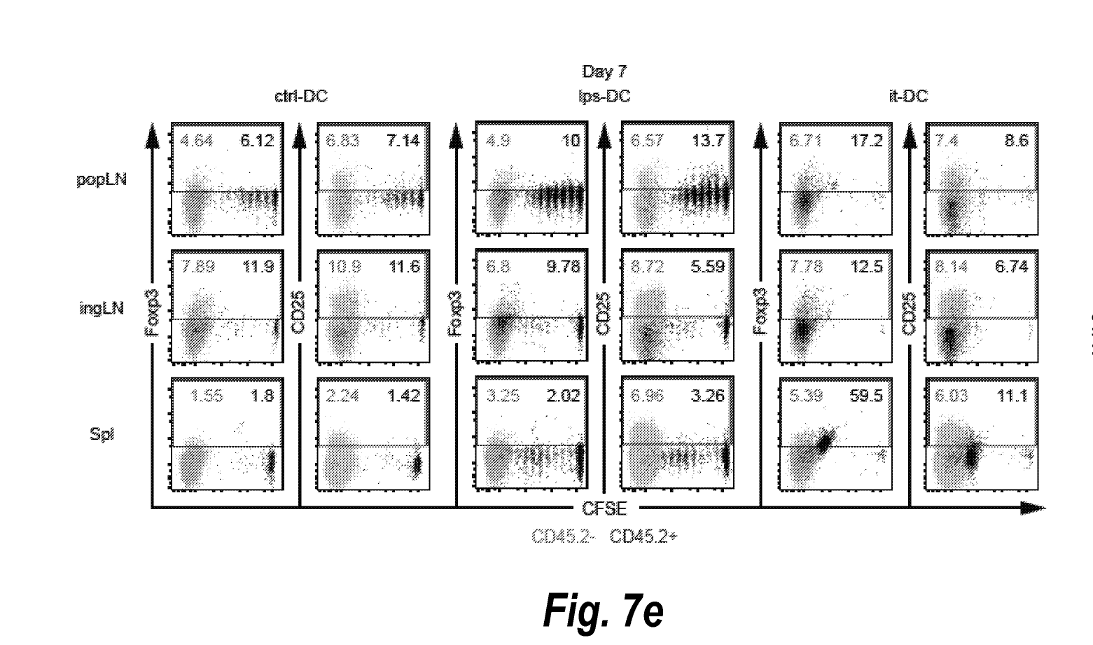

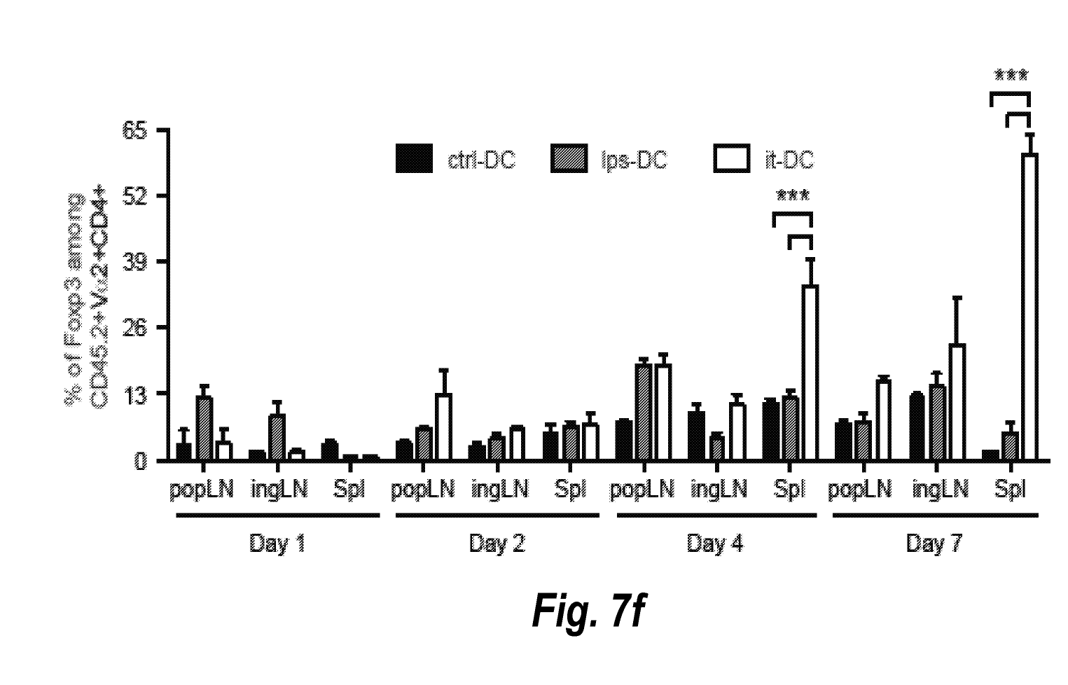

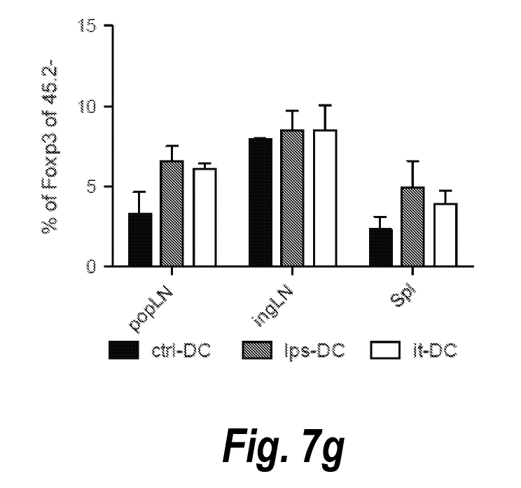

To assess how itDCs affect antigen-specific Tn in vivo, CD45.2+ OTII+RAG1−/− Tn were injected i.v. into CD45.1+C57BL/6 recipients followed by footpad injection of differentially treated CD45.2+ DC loaded with OVAp. Tn were isolated as previously described from Rag1−/−xOTII animals. DC were isolated and treated as described in Example 1h in the presence of OVAp. Different types of DC were injected in the hind footpad subcutaneously, whereas Tn cells were injected intravenously. Three animals per group were sacrificed at the time points indicated and their Bone Marrow (BM), popliteal (draining the site of injection) and inguinal lymph nodes (popLN and ingLN respectively) and the spleen (Spl) were harvested. Cell suspensions were analyzed for the presence of the transferred cells. A schematic representation of this experimental design is depicted in FIG. 7a.

The migratory capacities of all DC types were tested to exclude that any effect observed in vivo could be explained by differences in their homing. Therefore, DC from CD45.1+ animals inoculated with melanoma cell lines producing FLT3L were isolated and treated as previously described to obtain ctrlDC, itDC and lpsDC. These cells were differentially labeled with different dyes or cell trackers to detect their presence in the indicated organs after intravenous (FIG. 7b) or foot pad sub-cutaneous injections (not shown). No significant differences among the groups could be detected after their adoptive transfer a different time points (1 to 3 days, not shown).

Early time points are shown in FIG. 7c, which depicts cells from the popLN. Left panels show CD4+7AAD− cells, whereas right panels show CD4+Vα2+7AAD− cells. Percentages in the left panels (black text) refer to CD4+CD45.2+Vα2+7AAD− adoptively transferred Tn. cells. Right panels of FIG. 7c were gated on CD4+Vα2+7AAD− cells. Percentages refer to endogenous CD45.2− T cells (gray) and adoptively transferred CD45.2+(black) Tn. FIG. 7d shows a quantification of the percentage of transferred CD45.2+ cells in the CD4+Vα2+ fraction. FIG. 7e depicts representative plots of three independent experiments of the endogenous CD45.2− (gray) and adoptively transferred CD45.2+ (black) CD4+Vα2+ cells in the popLN, ingLN and Spl of animals 7 days after injection of DC (late time points). The compiled data of FIGS. 7c and 7d is shown in FIG. 7f. The quantification on FIG. 7g show the proportions of Foxp3+ cells among the endogenous cells (CD45.2−). These data show that the induction of Foxp3 after injection of itDC is restricted to antigen-specific T cells, i.e., there is not a polyclonal increase of Treg.

All p values were calculated using a Bonferroni post-test of a regular two-way ANOVA test (*=p<0.05, **=p<0.01, ***=p<0.001).

These results indicate that injection of itDC has profound consequences for the makeup of T cell repertoire in healthy animals. Injection of non-itDC led to a robust activation and proliferation of antigen-specific Tn, whereas itDC injection had the opposite effect, i.e., diminishing the quantity of these Tn and inducing Foxp3 expression in the remaining cells. A drop in OTII+ cell numbers was evident in all tissues of recipients of induced tolerogenic DC starting at day 2. The effect became more pronounced on days 4 and 7, when LPS treated and control DCs, but not induced tolerogenic DCs induced robust OTII+ T cell proliferation. The induction of Foxp3 expression occurred later, starting on day 5. This effect was antigen specific since endogenous Vα2+CD45.2− Treg remained unaltered (FIG. 7g).

### Example 8

### Effects of itDC Administration on the Course of a Disease: Mouse Models of Autoimmunity, Allogenic Responses and Allergic Asthma

Model of autoimmunity: Experimental Autoimmune Encephalomyelitis (EAE)

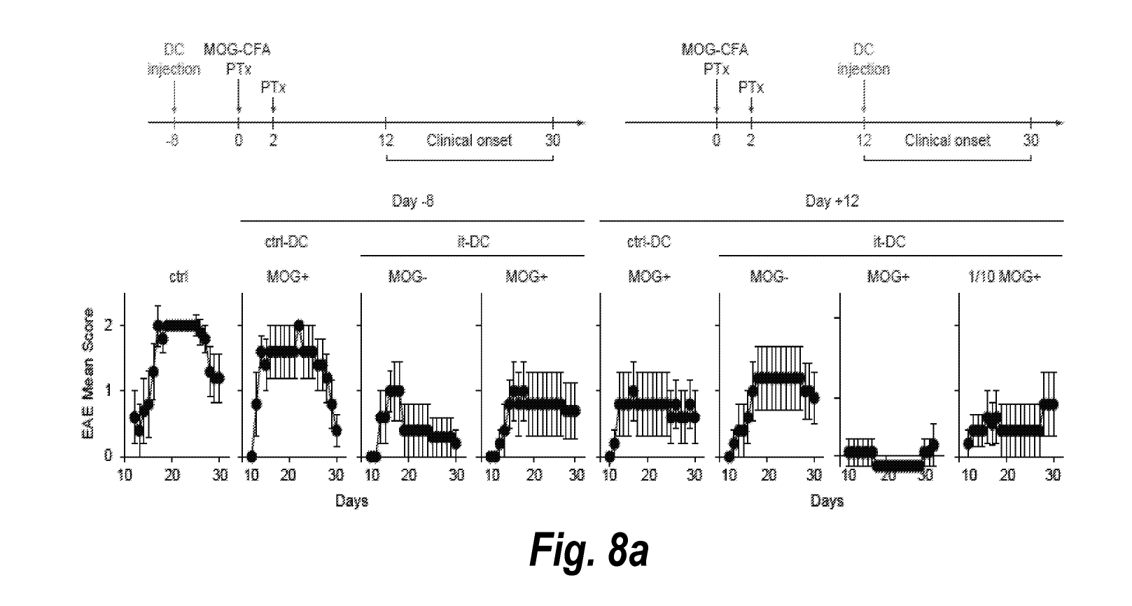

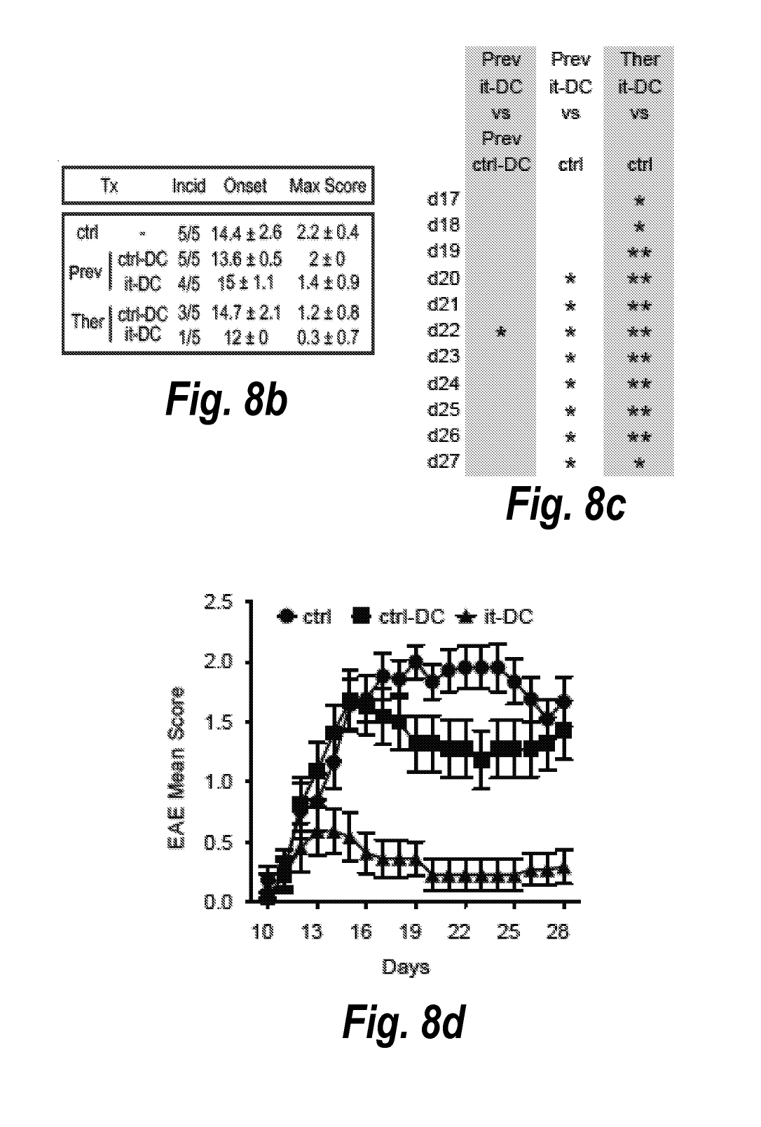

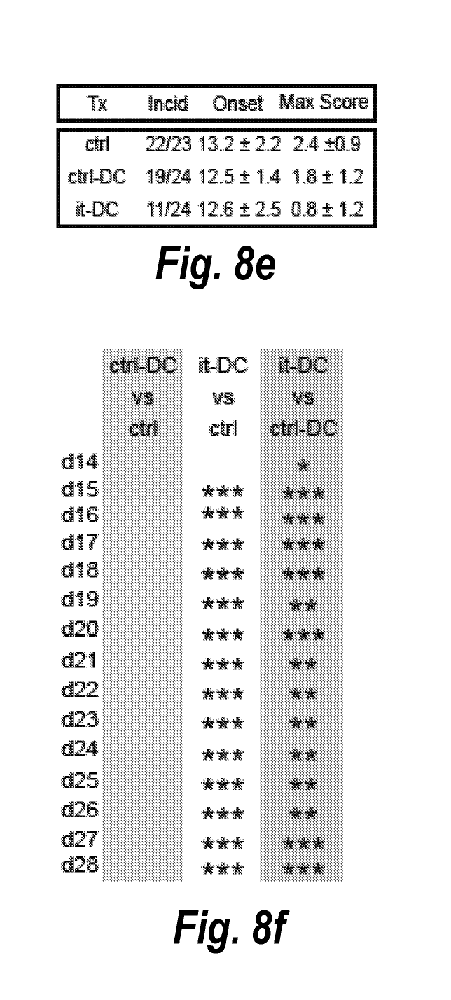

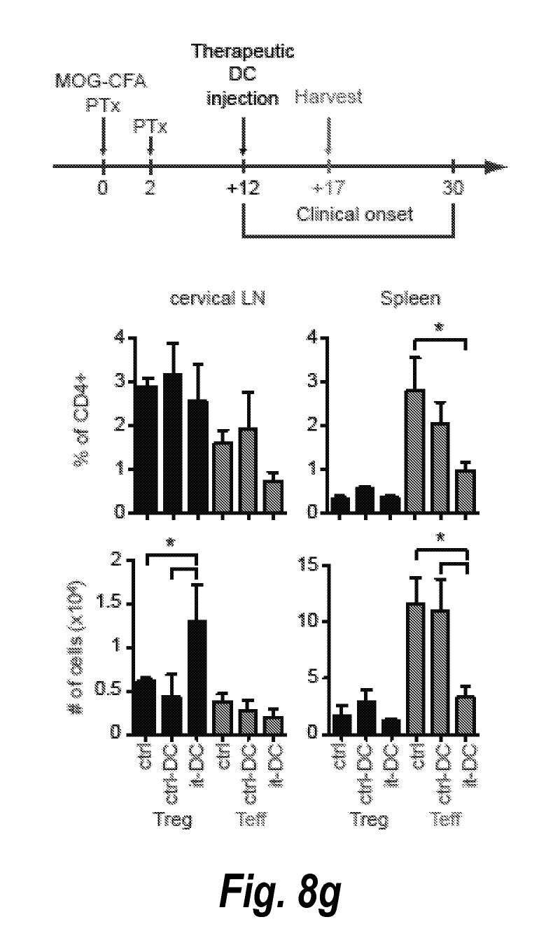

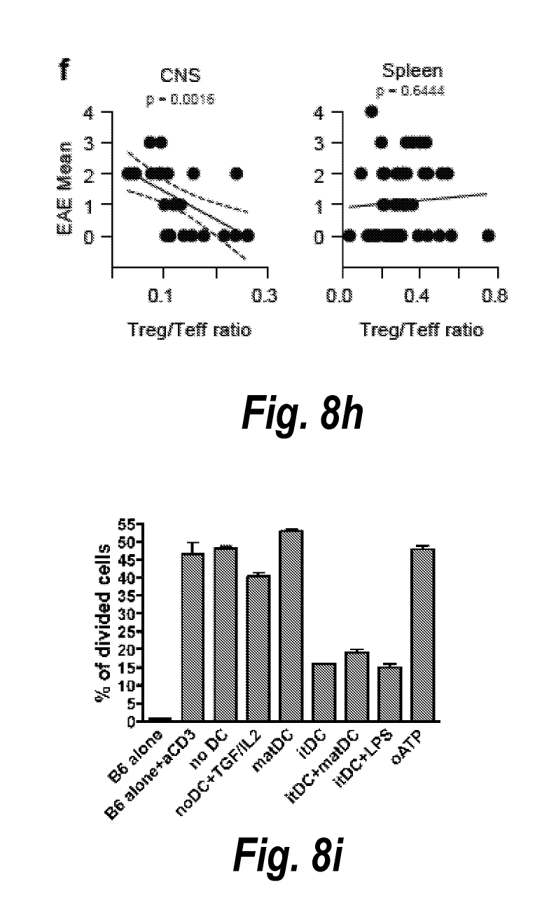

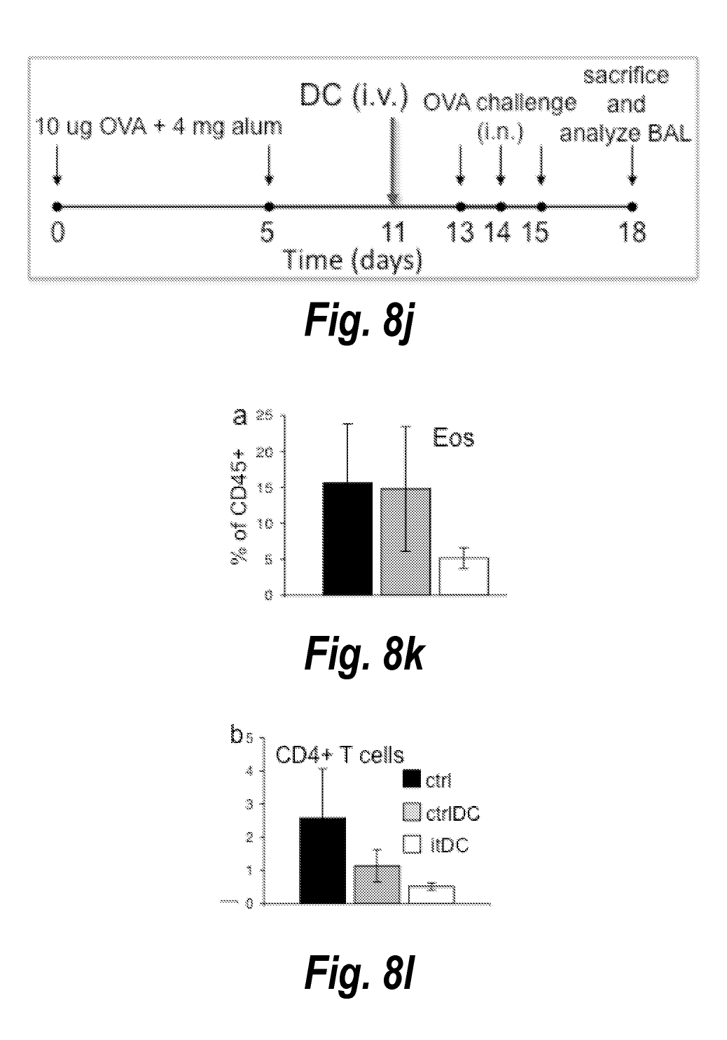

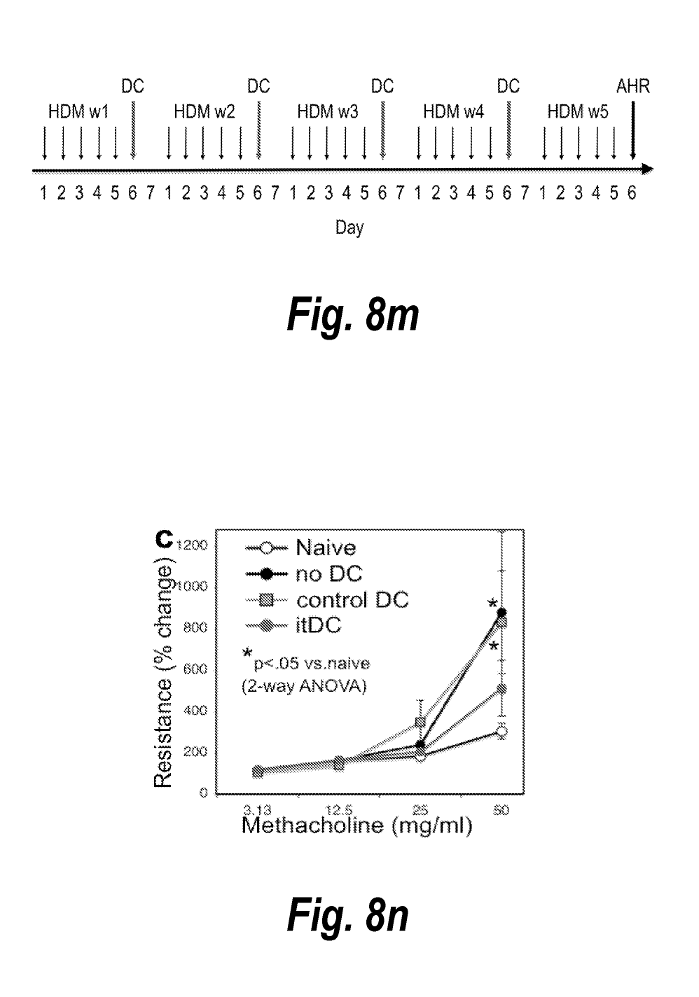

C57/BL6 animals were induced with EAE following standard protocols by injecting 100 μg/mouse of MOG₃₅₋₅₅ emulsified in CFA and 100 ng/mouse PTx at day 0 and 2. Eight days previous (Preventive, Day −8) or twelve days after (Therapeutic, Day +12) immunization, the animals were injected intravenously with 10⁷ DC, as shown in FIG. 8a (top). 10⁷ DC were treated as indicated in Example 1b, and loaded with the same peptide used to induce EAE (MOG+) or not (MOG−). In some conditions 1/10 of the cells were injected (10⁶, 1/10 MOG+). Scoring was done from the onset of the disease (day 12). A direct comparison of DC treatment using the preventive (Prev) or therapeutic protocol (Ther) is presented in FIG. 8b and FIG. 8c. Treatment (Tx), Incidence (Incid), maximal score (Max Score). FIG. 8c shows the statistical significance (p value) between EAE treatment groups (*=p<0.05, **=p<0.01, ***=p<0.001).

FIGS. 8d, 8e and 8f show the same parameters of compiled data of 5 independent experiments (5 animals per group, 24 animals per group total).

These results show that treatment with itDC can require that the DCs present the specific antigen (MOG+). Treatment at the peak of the disease is more effective than prophylactic intervention in this EAE model.

To further explore the effect of itDC on EAE, animals were immunized, treated therapeutically as previously described, and sacrificed at day 5 after DC injection. Cervical LN and spleens were harvested and cells were restimulated with MOG₃₅₋₅₅ overnight. Staining for regulatory T cells (Treg, CD25+Foxp3+ and/or IL10+ and/or TGF3+) and effector T cells (Teff, IFNγ+ and/or IL-17+) were performed. FIG. 8g shows the percentage and total number of Treg and Teff cells among CD4+ live single cells after the therapeutic DC injection.

In order to correlate T cell phenotype with EAE score, animals were randomized at the end of the experiment to harvest their organs and surveyed for the presence of Teff cells (IL-17+ and/or IFNγ+ and/or TNFα) or Treg cells (CD25+Foxp3+ and/or IL-10+ and/or TGFβ) after restimulation with MOG₃₅₋₅₅ overnight. Spleen, central nervous system (CNS, brain and spinal cords), lymph nodes (cervical, brachial, inguinal, mesenteric, not shown) and bone marrow were harvested. FIG. 8h shows correlation plots where each dot represents the value of the mean EAE score plotted against the Treg/Teff ratio per animal. Solid lines represent the best-fit line whereas dotted lines show the 95% confidence band. These results show that animals in recovery (after DC injection or not) have a highly significant correlation between a high Treg/Teff ratio and lower score disease.

All p values were calculated using a Bonferroni post-test of a regular two-way ANOVA test (*=p<0.05, **=p<0.01, ***=p<0.001).

Together, these data demonstrate that itDC treatment effectively halts the progress of EAE likely through the elimination of Teff cells that induce and maintain the disease, and by stimulating Treg that ensure tolerance towards the self-antigen.

### Model of Allogenic Reactions: Mixed Leukocyte Reaction (MLR)

Splenic untreated Balb/c DC (presenters) were mixed with CFSE-labeled C57BL/6 T cells (responders; 10⁵ cells of each population). Differentially treated F1 (BalbxB6) DC (10⁴/well) were added to some wells. After 4 days, the proliferation of responders was analyzed by FACS. C57BL/6 responders alone (without presenters) were used as negative (no stimulation) or positive controls (with anti-CD3 stimulation). The x-axis labels in FIG. 8i refer to the presence and conditions of F1 DCs in cultures. Note that itDC were tolerogenic whether or not they had been activated with LPS and in the presence or absence of additional LPS-matured DC (matDC). In a separate experiment (not shown), itDC from Balb/c and C57BL/6 donors also attenuated T cell proliferation, but to a lesser degree than itDC from F1 donors.

### Model of Allergic Asthma

To test the effect of itDC in allergic asthma. C57/BL6 mice were sensitized and challenged with OVA as described in materials and methods. On D11, animals were left untreated (ctrl) or injected i.v. with 10⁷ OVAp-loaded ctrlDC or itDC. Animals were sacrificed on D18 (FIG. 8j). BAL fluid was evaluated for eosinophils (CD11b⁺Gr1^{lo}SSC-A^{hi}, FIG. 8k) and CD4⁺ T cells (FIG. 81). Three days after the last challenge, airway eosinophilia and CD4+ lymphocytes in bronchoalveolar fluid were reduced in itDC recipients, consistent with the idea that itDC may be useful to treat pathologic responses to exogenous antigens. FIG. 8n shows the results of an experiment in which mice were sensitized to house dust mite (HDM, see materials and methods) 5 days a week intranasaly every week for 5 weeks (HDM) and methacholine-induced airway hyperresponsiveness (AHR, as described in materials and methods) was measured on D25 (FIG. 8m). HDM-pulsed itDC injections after every set of HDM stimulation (on day 6 of every week, total 4 treatments) attenuated the AHR response, suggesting that itDC exert therapeutic effects irrespective of genetic background (B6 and Balb/c) and at both the level of inflammation and respiratory function. It is important to note here that HDM is a crude extract from these acarids and represents a complex mixture of antigens as opposed to the protocols used up to now with peptides and purified proteins. The results presented here suggest that itDC can elicit a tolerogenic effect using such preparations consistent with the idea that they can process and present relevant antigens in the course of the immune disorder.

### Example 9

### No Effect of Direct Treatment with Rapamycin on the Proportions of Foxp3+ T Cells in the Lymph Node In Vivo

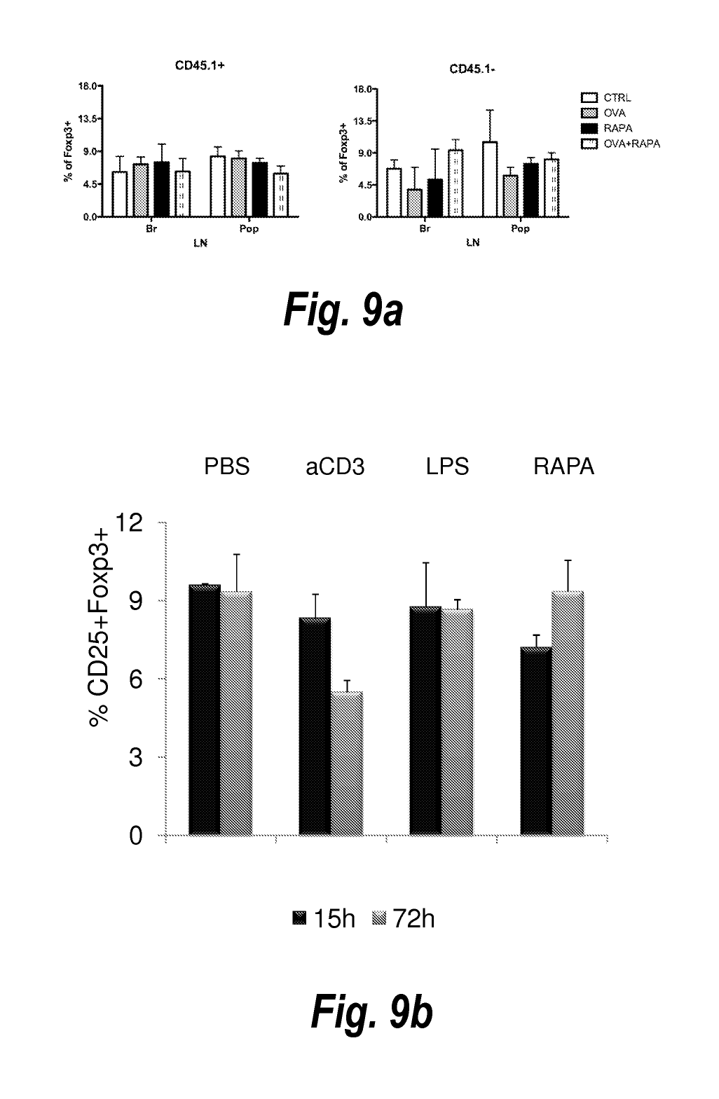

Tn were isolated from OTIIxCD45.1 donors and injected into CD45.2+ recipients. The next day the animals were injected into the hind footpad with control saline (PBS), OVAp alone (10 ug), rapamycin alone (1 ug) or a combination of both. Five days later the draining popliteal and non-draining brachial lymph nodes were extracted and analyzed for the presence of CD25+Foxp3+ among the transferred (CD45.1+) or the endogenous (CD45.1−) Vα2+CD4+ cells. As shown FIG. 9a no effect could be observed on the adoptively transferred or endogenous T cells.

Similar experiments were performed to evaluate the effect of direct treatment with rapamycin on the proportions of Foxp3+ T cells in the lymph node in vivo. Tn were isolated from CD45.1+ donors and injected into CD45.2+ recipients. The next day the animals were injected into the hind footpad with control saline (PBS), activating anti-CD3 antibody (10 ug), lipopolysaccharide from E. coli (LPS, 1 ug) and rapamycin (1 ug). At the indicated times the draining popliteal lymph nodes were extracted and analyzed for the presence of CD25+Foxp3+ among Vα2+CD4+CD45.1+ cells at the times indicated. As shown in FIG. 9b no differences in the amount of Foxp3 could be detected.

### Example 10

### Screening of Additional Compounds to Induce Tolerogenic DC

a) oATP Induces Tolerogenic Function on DC.

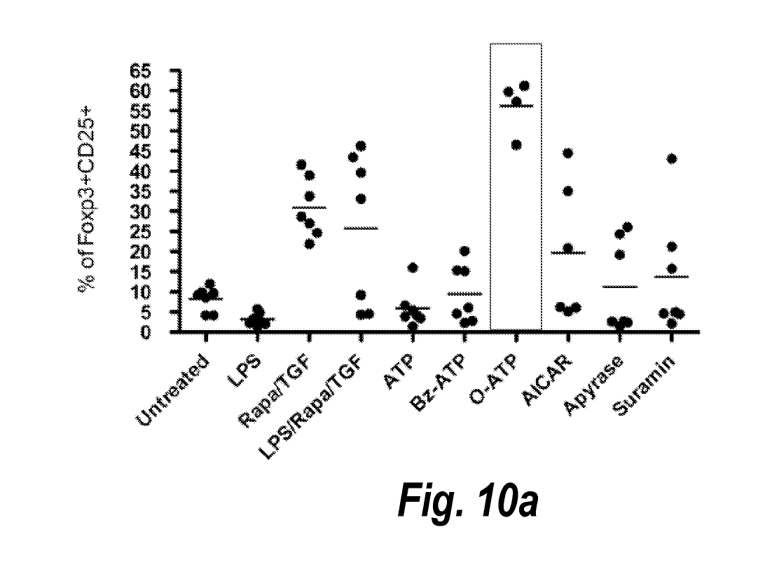

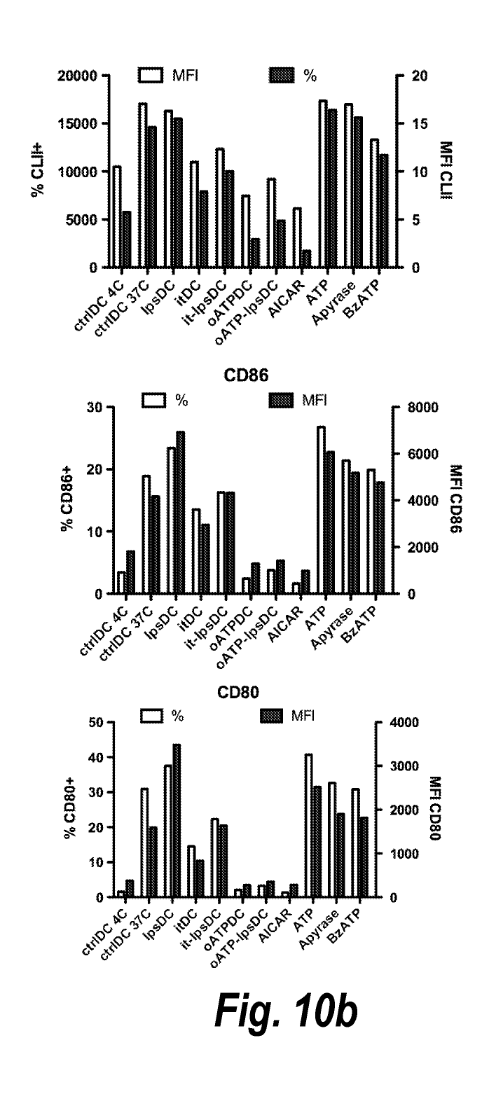

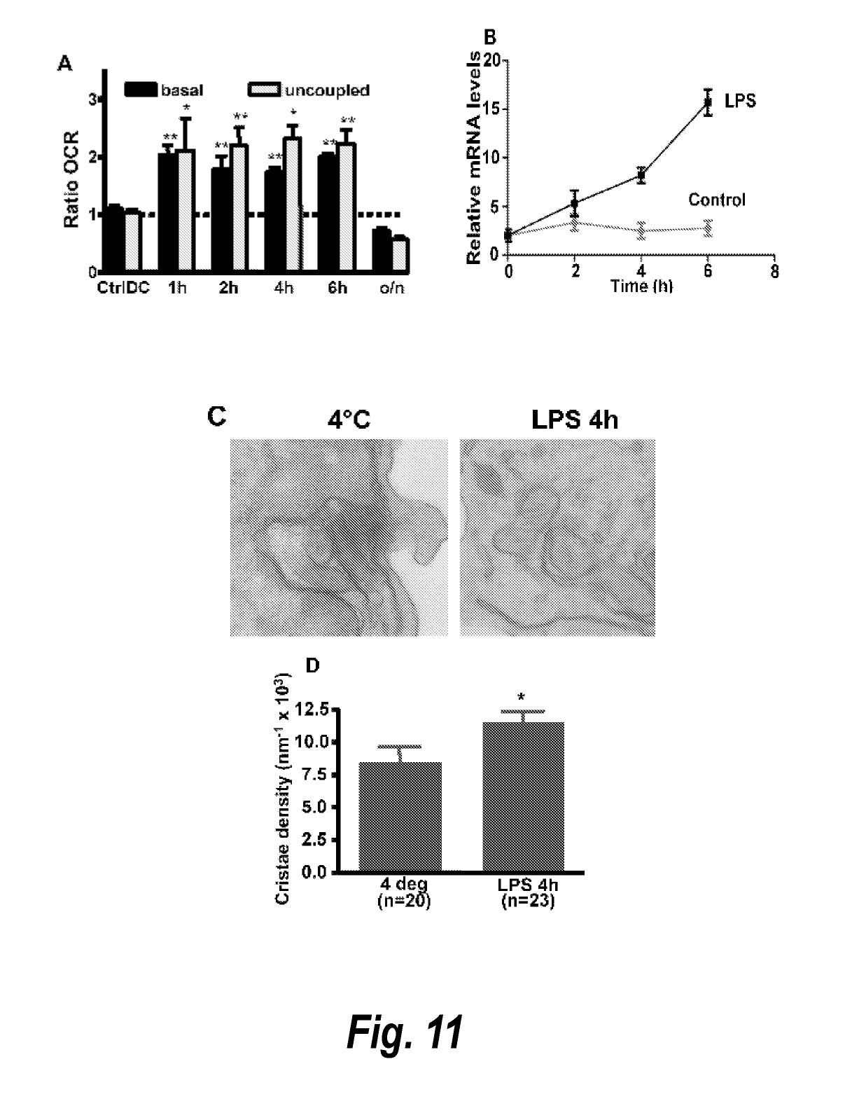

DC were purified by MACS using double positive selection with anti-CD11c-coupled beads (around 98% pure) from the spleens of normal C57/BL6 animals, or animals inoculated nine days before with FLT3L-producing melanomas (see materials and methods). DC were incubated for 2 hours under tissue culture conditions (37° C., 5% CO2) with LPS (1 μg/ml), rapamycin (10 nM) and TGFβ (2 ng/ml, Rapa/TGF); the purinergic receptor agonists ATP (1 mM) or Bz-ATP (100 μM); purinergic receptor antagonists oxidized ATP (oATP, 100 μM) or suranim (100 μM); or the adenosine monophosphate kinase (AMPK) stimulator AICAR (100 μM) or the enzyme Apyrase (200 U/ml) that catalyses ATP. Hen Ovalbumin protein (OVAp, 100 μg/ml) was used in this incubation to load DC. Following treatment, DC were washed extensively (up to 6×). Tn were isolated from OTIIxFoxp3GFP reporter x transgenic mice by FACS sorting of CD4+CD62Lhigh CD44low CD25− Foxp3GFP− cells (Tn Foxp3−). CD45.1+ or CD45.2+ animals were used as donors of DC and Tn (interchanged across experiments). FIG. 10a shows the percentages of Foxp3 and CD25 expressing cells identified by measuring GFP expression of CD4+CD11c−7AAD− cells after 5 days in coculture with treated DC. FIG. 10a summarizes results from 5 different independent experiments, where each dot represents the average of triplicates. This screen of DC conditioning reveals that treatment of DC with oATP significantly imprints DC with tolerogenic potential. This capacity matches or exceeds that of the combination of rapamycin and TGF3.

b) Blockade of Maturation by oATP and AICAR:

DC were isolated and kept for 6 hours at 4 degrees (ctrlDC 4C) or culture to produce ctrlDC (37C), lpsDC itDC and it/lpsDC. Simultaneously cells were treated with the purinergic receptor agonists ATP (1 mM) and Bz-ATP (100 μM), purinergic receptors antagonists oxidized ATP (oATP, 100 μM) and suranim (100 μM), the adenosine monophosphate kinase (AMPK) stimulator AICAR (100 μM) and the enzyme Apyrase (200 U/ml) that catalyses ATP. Cells were harvested after 6 hours and the expression of the indicated markers was assessed by FACS. These experiments show that oATP and AICAR block DC maturation.

### Example 11

### The Effect of DC Activation on Mitochondria

The effect of the activation of DC on the O₂ consumption rate (OCR) was tested to attempt to correlate increased OCR with increased immunogenicity. In fact, hours after receiving a maturation signal, control DCs dramatically increased their OCR, but this increase did not occur in R+T treated tolerogenic dendritic cells. Panel (a) of FIG. 1k shows mitochondrial respiration (the oxygen consumption rate) in control and LPS-stimulated dendritic cells over time. Both basal and uncoupled OCR ratios increased after LPS. After overnight (o/n) maturation, OCR of lpsDC was reduced.

In panel B, the expression kinetics of PGC-1α was measured based on the fact that the YY1-PGC-1α transcriptional complex is known to control mitochondrial oxidative function. Real-time PCR was performed on untreated (control) and LPS-treated DC and normalized to GAPDH. In addition, the transmission electron microscopy of mitochondria was performed in control and LPS-treated DCs and is shown in panel C. Panel D shows in increase in the density of mitochondrial christae in activated DCs determined using ImageJ software as: total cristae length (nm) in a mitochondrium:area (nm²) of the same mitochondrium.

### Example 12

### OCR and Immunogenicity of DC

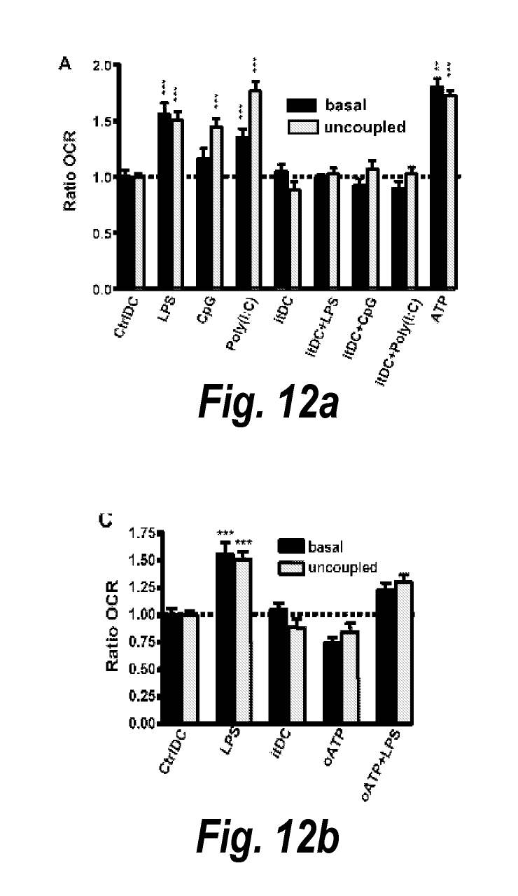

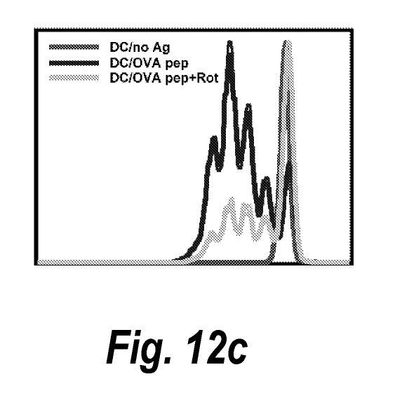

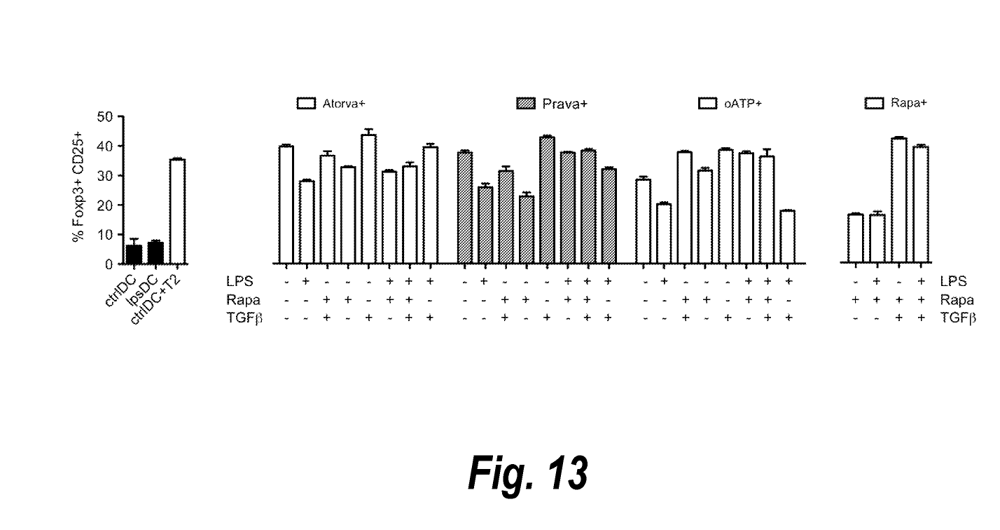

High concentrations of extracellular ATP (ATP, which activates the inflammasome as well as mTOR, which controls both intracellular ATP levels and ATP release) enhanced OCR levels in DCs as well as TLR agonists (FIG. 12a). Given the potency of oATP to evoke itDC function (FIG. 10a) and block maturation (FIG. 10b), it was next tested whether blocking ATP receptors (ATPR) using this reagent reduces DC OCR (FIG. 12b). Cells that were treated with oATP demonstrate reduced OCR as compared to their control whether they were co-treated with LPS or not. The effect of direct inhibition of mitochondrial respiration, by an inhibitor of mitochondrial respiratory complex I, rotenone, was tested. As shown in FIG. 12c, inhibition of electron transport in mitochondria by rotenone blocks DC immunogenicity. CFSE labeled OT-II T cells were incubated with OVA peptide pulsed DC that had been matured with LPS alone or LPS plus 1 mM rotenone. Control DC were treated with LPS without OVA pulsing. T cell proliferation was analyzed by CFSE dilution on D3. Antigen pulsed DC that had been treated with LPS in the presence of rotenone, induced much less T cell proliferation than control DC. By contrast, there was no difference between DC subsets in maturation markers (CD80, C86, MHC-II) and migration to CCL21 in transwell chemotaxis assays (not shown), indicating that rotenone was not toxic to DC.

### Example 13

### Induction of Tolerogenic Function by Treatment of DC with Statins

DC from normal animals were isolated, loaded with OVAp, treated as in previous experiments to produce ctrlDC, lpsDC and itDC. Additionally cell were treated with Atorvastatin (Atorva, 10 uM), Pravastatin (Prava, 50 uM) or oATP (100 μM) in combination or not with rapamycin, TGFβ and LPS as indicated. These cells were used to activate OTII+Foxp3GFP−CD45.1+ Tn and after 5 days in culture the presence of CD25+Foxp3+ among Vα2+CD4+CD45.1+ cells was assessed by FACS. Positive controls were cell cultures in which additional TGFβ and IL-2 were added (ctrlDC+T2). These experiments show that treatment with statins elicit tolerogenic function on DC similar to the combination of rapamycin and TGFβ that is resistant to the presence of the presence of the TLR agonist LPS.

### EQUIVALENTS

The invention has been described herein with reference to certain examples and embodiments only. No effort has been made to exhaustively describe all possible examples and embodiments of the invention. Indeed, those of skill in the art will appreciate that various additions, deletions, modifications and other changes can be made to the above-described examples and embodiments, without departing from the intended spirit and scope of the invention as recited in the following claims. It is intended that all such additions, deletions, modifications and other changes be included within the scope of the following claims.

1-95. (canceled)

96. A composition comprising induced tolerogenic dendritic cells (DCs) characterized by antigen specific tolerance induction, wherein the tolerogenic DCs possess at least one of the following characteristics:
(i) the ability to convert naïve T cells to Foxp3⁺ T regulatory cells ex vivo;
(ii) the ability to delete effector T cells ex vivo; (iii) the ability to increase expression of co stimulatory molecules but retain their tolerogenic phenotype upon stimulation with at least one TLR agonist ex vivo; and
(iv) the ability to remain repirostatic upon stimulation with at least one TLR agonist ex vivo.

97. The composition of claim 96, wherein the DCs express class II molecules, and wherein at least a portion of the class II molecules are bound to a plurality of antigenic peptides derived from an antigen to which T cell tolerance is desired.

98. The composition of claim 96 produced by a method comprising contacting a starting population of cells comprising dendritic cells or dendritic cell precursors ex vivo with at least one agent that has at least one of the following activities,
(i) promotes respirostatic tolerance;
(ii) disrupts mitochondrial electron transport;
(iii) induces the DCs to increase expression of costimulatory molecules but retain their tolerogenic phenotype upon stimulation with at least one TLR agonist; and
(iii) cause the DCs to induce Foxp3 expression in naïve T cells.

99. The composition of claim 98, wherein the at least one agent is selected from the group consisting of:
i) an mTOR inhibitor and a TGFβ agonist;
ii) a statin;
iii) an mTOR inhibitor and a statin;
iv) an mTOR inhibitor, a TGFβ agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ agonist;
ix) a purinergic receptor antagonist, an mTOR inhibitor, a TGFβ agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

100. The composition of claim 98, wherein the starting population of cells comprising dendritic cells or dendritic cell precursors is contacted with the agent ex vivo for less than 10 h.

101. The composition of claim 98, wherein the method further comprises contacting the starting population of cells or the induced tolerogenic DCs with at least one agent that promotes differentiation of DCs.

102. The composition of claim 98, wherein the method further comprises contacting the induced tolerogenic DCs or the starting population of cells with an antigen to which tolerance is desired.

103. The composition of claim 98, wherein the method further comprises contacting the induced tolerogenic dendritic cells with effector T cells.

104. A method of treating a disease or disorder mediated by an unwanted immune response comprising administering to a subject in need thereof a composition comprising induced tolerogenic dendritic cells (DCs) characterized by antigen specific tolerance induction.

105. The method of claim 104, wherein the disease or disorder is associated with inflammation or autoimmunity.

106. A method of producing a population of cells comprising the induced tolerogenic DCs, the method comprising contacting a starting population of cells comprising dendritic cells or dendritic cell precursors ex vivo with at least one agent that has at least one of the following activities,
(i) promotes respirostatic tolerance;
(ii) disrupts mitochondrial electron transport;
(iii) induces the DCs to increase expression of costimulatory molecules but retain their tolerogenic phenotype upon stimulation with at least one TLR agonist; and
(iii) cause the DCs to induce Foxp3 expression in naïve T cells
wherein the DCs are characterized by antigen specific tolerance induction.

107. The method of claim 106, wherein the at least one agent is selected from the group consisting of:
i) an mTOR inhibitor and a TGFβ agonist;
ii) a statin;
iii) an mTOR inhibitor and a statin;
iv) an mTOR inhibitor, a TGFβ agonist, and a statin;
v) a purinergic receptor antagonist;
vi) a purinergic receptor antagonist and a statin;
vii) a purinergic receptor antagonist and an mTOR inhibitor;
viii) a purinergic receptor antagonist, an mTOR inhibitor and a TGFβ agonist;
ix) a purinergic receptor antagonist, an mTOR inhibitor, a TGFβ agonist and a statin;
x) an agent which disrupts mitochondrial electron transport in the DCs;
xi) an agent which disrupts mitochondrial electron transport in the DCs and an mTOR inhibitor;
xii) an agent which disrupts mitochondrial electron transport in the DCs and a statin;
xiii) an agent which disrupts mitochondrial electron transport in the DCs, an mTOR inhibitor, and a TGFβ agonist;
xiv) an agent which disrupts mitochondrial electron transport in the DCs, an mTOR inhibitor, a TGFβ agonist, and a statin.

108. The composition of claim 106, wherein the starting population of cells comprising dendritic cells or dendritic cell precursors is contacted with the agent ex vivo for less than 10 h.

109. The method of claim 106, wherein the method further comprises contacting the starting population of cells or the induced tolerogenic DCs with at least one agent that promotes differentiation of DCs.

110. The method of claim 106, wherein the method further comprises contacting the induced tolerogenic DCs or the starting population of cells with an antigen to which tolerance is desired.

111. The composition of claim 106, wherein the method further comprises contacting the induced tolerogenic dendritic cells with effector T cells.

112. A method of producing a composition comprising induced immunogenic dendritic cells, the method comprising contacting a starting population of cells comprising dendritic cells or dendritic cell precursors ex vivo with a stimulus which increases oxygen consumption in the dendritic cells, to thereby produce a composition comprising induced immunogenic dendritic cells, optionally wherein the induced immunogenic dendritic cells comprise fully differentiated dendritic cells.

113. A method of treating a disease or disorder mediated by lack of a desired immune response to an antigen, the method comprising administering to a subject in need thereof a composition comprising induced immunogenic dendritic cells in an amount sufficient to increase T effector cell responsiveness to an antigen.

114. A method of identifying an agent which promotes antigen-specific tolerance, comprising contacting a population of cells comprising dendritic cells or dendritic cell precursors with a test agent to obtain treated cells, measuring effect of the agent on mitochondrial activation, wherein agents that prevent or reverse mitochondrial activation in the treated cells as compared to an appropriate control are selected as candidate agents for promoting antigen-specific T cell tolerance.

115. A method of identifying an agent which promotes an antigen-specific T effector cell response, comprising contacting a population of cells comprising dendritic cells or dendritic cell precursors with a test agent, measuring the effect of the agent on mitochondrial activation, wherein agents that increase mitochondrial activation as compared to an appropriate control are selected as candidate agents for promoting an antigen-specific T effector T cell response.
